(19) United States
(12) Patent Application Publication
(10) Pub. No.: US 2018/0325902 A1
(43) Pub. Date: Nov. 15, 2018

(54) COMPOSITIONS AND METHODS RELATING TO CANCER

(71) Applicant: The Penn State Research Foundation, University Park, PA (US)

(72) Inventors: Gavin P. Robertson, Hummelstown, PA (US); Raghavendragowda Gowda Chandagalu Doreswamy, Hershey, PA (US); Omer F. Kuzu, Hershey, PA (US); Arati K. Sharma, Hummelstown, PA (US); Gregory Kardos, Phoenixville, PA (US); Subbarao V. Madhunapantula, Karnataka (IN); Mohammed A. Noory, Hershey, PA (US); Joseph J. Drabick, Palmyra, PA (US)

(21) Appl. No.: 15/774,196

(22) PCT Filed: Nov. 7, 2016
(86) PCT No.: PCT/US16/00097

§ 371 Date: May 7, 2018

Related U.S. Application Data
(60) Provisional application No. 62/252,132, filed on Nov. 6, 2015.
(60) Provisional application No. 62/281,397, filed on Jan. 21, 2016.

(51) Int. Cl. A61K 31/519; A61P 35/02

(52) U.S. Cl. CPC A61K 31/519; A61K 31/517; A61K 2300/00; A61P 35/02

(57) ABSTRACT

Compositions and methods continue to be required for treatment of cancer. Compositions and methods according to aspects of the present invention relate to inhibition of a combination of kinases for treatment of cancer, specifically inhibition of both AKT and WEE1 kinases for treatment of cancer in a human subject. Synergistic effects of combination compositions and treatments including administration of an AKT inhibitor and a WEE1 inhibitor are unexpectedly found as described herein.

### REFERENCE TO RELATED APPLICATIONS

This application claims priority to U.S. Provisional Patent Application Ser. Nos. 62/252,132, filed Nov. 6, 2015 and 62/281,397, filed Jan. 21, 2016, the entire content of both of which is incorporated herein by reference.

### GOVERNMENT SPONSORSHIP

This invention was made with government support under Grant Nos. CA136667; CA138634; and CA200284 awarded by the National Institutes of Health. The government has certain rights in the invention.

### FIELD OF THE INVENTION

Compositions and methods according to general aspects of the present invention relate to inhibition of a combination of kinases for treatment of cancer. Compositions and methods according to specific aspects of the present disclosure relate to inhibition of a AKT and WEE1 kinases for treatment of cancer in a human subject.

### BACKGROUND OF THE INVENTION

Incidence and mortality rates for cancers, including malignant melanoma, continue to rise annually.

For example, an estimated 76,000 new cases of melanoma and over 9,000 deaths will occur this year in the United States. Advanced-stage metastatic melanoma carries a poor prognosis, with an overall median survival of 2 to 8 months, and with only 5% of patients surviving beyond 5 years.

Currently, significant efforts are being made to improve long-term management of patients with cancer using with newer drugs and rational strategies for combination-based therapy but there is a continuing need for compositions and methods for treatment of cancer.

### SUMMARY OF THE INVENTION

Compositions are provided according to aspects of the present invention which include an AKT inhibitor and a WEE1 inhibitor.

Compositions are provided according to aspects of the present invention which include a WEE1 inhibitor and an AKT inhibitor selected from the group consisting of: AZD5363, GDC0068, a combination of AZD5363 and GDC0068, a pharmaceutically acceptable salt, hydrate, amide or ester of any thereof, and an siRNA directed to AKT.

Compositions are provided according to aspects of the present invention which include an AKT inhibitor and a WEE1 inhibitor selected from the group consisting of: MK1775, a pharmaceutically acceptable salt, hydrate, amide or ester thereof, and an siRNA directed to WEE1.

Compositions are provided according to aspects of the present invention which include an AKT inhibitor selected from the group consisting of: AZD5363, GDC0068, a combination of AZD5363 and GDC0068, a pharmaceutically acceptable salt, hydrate, amide or ester of any thereof, and an siRNA directed to AKT; and a WEE1 inhibitor selected from the group consisting of: MK1775, a pharmaceutically acceptable salt, hydrate, amide or ester thereof, and an siRNA directed to WEE1.

Compositions are provided according to aspects of the present invention which include an AKT inhibitor and a WEE1 inhibitor and which exclude CHK1 inhibitors. Compositions are provided according to aspects of the present invention which include an AKT inhibitor and a WEE1 inhibitor and which exclude mTOR inhibitors. Compositions are provided according to aspects of the present invention which include an AKT inhibitor and a WEE1 inhibitor and which exclude both CHK1 inhibitors and mTOR inhibitors.

Compositions are provided according to aspects of the present invention which include a WEE1 inhibitor and an AKT inhibitor selected from the group consisting of: AZD5363, GDC0068, a combination of AZD5363 and GDC0068, a pharmaceutically acceptable salt, hydrate, amide or ester of any thereof, and an siRNA directed to AKT; wherein the compositions exclude CHK1 inhibitors, mTOR inhibitors or both CHK1 inhibitors and mTOR inhibitors.

Compositions are provided according to aspects of the present invention which include an AKT inhibitor and a WEE1 inhibitor selected from the group consisting of: MK1775, a pharmaceutically acceptable salt, hydrate, amide or ester thereof, and an siRNA directed to WEE1; wherein the compositions exclude CHK1 inhibitors, mTOR inhibitors or both CHK1 inhibitors and mTOR inhibitors.

Compositions are provided according to aspects of the present invention which include an AKT inhibitor selected from the group consisting of: AZD5363, GDC0068, a combination of AZD5363 and GDC0068, a pharmaceutically acceptable salt, hydrate, amide or ester of any thereof, and an siRNA directed to AKT; and a WEE1 inhibitor selected from the group consisting of: MK1775, a pharmaceutically acceptable salt, hydrate, amide or ester thereof, and an siRNA directed to WEE1; wherein the compositions exclude CHK1 inhibitors, mTOR inhibitors or both CHK1 inhibitors and mTOR inhibitors.

Compositions are provided according to aspects of the present invention which include an AKT inhibitor, a WEE1 inhibitor; and a pharmaceutically acceptable carrier.

Compositions are provided according to aspects of the present invention which include a WEE1 inhibitor; an AKT inhibitor selected from the group consisting of: AZD5363, GDC0068, a combination of AZD5363 and GDC0068, a pharmaceutically acceptable salt, hydrate, amide or ester of any thereof, and an siRNA directed to AKT; and a pharmaceutically acceptable carrier.

Compositions are provided according to aspects of the present invention which include an AKT inhibitor; a WEE1 inhibitor selected from the group consisting of: MK1775, a pharmaceutically acceptable salt, hydrate, amide or ester thereof, and an siRNA directed to WEE1; and a pharmaceutically acceptable carrier.

Compositions are provided according to aspects of the present invention which include an AKT inhibitor selected from the group consisting of: AZD5363, GDC0068, a combination of AZD5363 and GDC0068, a pharmaceutically acceptable salt, hydrate, amide or ester of any thereof, and an siRNA directed to AKT; a WEE1 inhibitor selected from the group consisting of: MK1775, a pharmaceutically acceptable salt, hydrate, amide or ester thereof, and an siRNA directed to WEE1; and a pharmaceutically acceptable carrier.

Compositions are provided according to aspects of the present invention which include an AKT inhibitor; a WEE1 inhibitor; and a pharmaceutically acceptable carrier wherein the composition exclude CHK1 inhibitors. Compositions are provided according to aspects of the present invention which include an AKT inhibitor, a WEE1 inhibitor; and a pharmaceutically acceptable carrier, wherein the composition exclude mTOR inhibitors. Compositions are provided according to aspects of the present invention which include an AKT inhibitor; a WEE1 inhibitor, and a pharmaceutically acceptable carrier, wherein the compositions exclude both CHK1 inhibitors and mTOR inhibitors.

Compositions are provided according to aspects of the present invention which include a WEE1 inhibitor; an AKT inhibitor selected from the group consisting of: AZD5363, GDC0068, a combination of AZD5363 and GDC0068, a pharmaceutically acceptable salt, hydrate, amide or ester of any thereof, and an siRNA directed to AKT; and a pharmaceutically acceptable carrier, wherein the compositions exclude CHK1 inhibitors, mTOR inhibitors or both CHK1 inhibitors and mTOR inhibitors.

Compositions are provided according to aspects of the present invention which include an AKT inhibitor, a WEE1 inhibitor selected from the group consisting of: MK1775, a pharmaceutically acceptable salt, hydrate, amide or ester thereof, and an siRNA directed to WEE1; and a pharmaceutically acceptable carrier, wherein the compositions exclude CHK1 inhibitors, mTOR inhibitors or both CHK1 inhibitors and mTOR inhibitors.

Compositions are provided according to aspects of the present invention which include an AKT inhibitor selected from the group consisting of: AZD5363, GDC0068, a combination of AZD5363 and GDC0068, a pharmaceutically acceptable salt, hydrate, amide or ester of any thereof, and an siRNA directed to AKT; a WEE1 inhibitor selected from the group consisting of: MK1775, a pharmaceutically acceptable salt, hydrate, amide or ester thereof, and an siRNA directed to WEE1; and a pharmaceutically acceptable carrier, wherein the compositions exclude CHK1 inhibitors, mTOR inhibitors or both CHK1 inhibitors and mTOR inhibitors.

Commercial packages are provided according to aspects of the present invention which include an AKT inhibitor and a WEE1 inhibitor, wherein the AKT inhibitor and WEE1 inhibitor are provided together in a single pharmaceutical formulation or in separate pharmaceutical formulations.

Commercial packages are provided according to aspects of the present invention which include an AKT inhibitor selected from the group consisting of: AZD5363, GDC0068, a combination of AZD5363 and GDC0068, a pharmaceutically acceptable salt, hydrate, amide or ester of any thereof, and an siRNA directed to AKT; and a WEE1 inhibitor, wherein the AKT inhibitor and WEE1 inhibitor are provided together in a single pharmaceutical formulation or in separate pharmaceutical formulations.

Commercial packages are provided according to aspects of the present invention which include a WEE1 inhibitor selected from the group consisting of: MK1775, a pharmaceutically acceptable salt, hydrate, amide or ester thereof, and an siRNA directed to WEE1; and an AKT inhibitor, wherein the AKT inhibitor and WEE1 inhibitor are provided together in a single pharmaceutical formulation or in separate pharmaceutical formulations.

Commercial packages are provided according to aspects of the present invention which include an AKT inhibitor selected from the group consisting of: AZD5363, GDC0068, a combination of AZD5363 and GDC0068, a pharmaceutically acceptable salt, hydrate, amide or ester of any thereof, and an siRNA directed to AKT; and a WEE1 inhibitor selected from the group consisting of: MK1775, a pharmaceutically acceptable salt, hydrate, amide or ester thereof, and an siRNA directed to WEE1, wherein the AKT inhibitor and WEE1 inhibitor are provided together in a single pharmaceutical formulation or in separate pharmaceutical formulations.

Methods of treating cancer in a subject in need thereof are provided according to aspects of the present invention which include administering a combination of an AKT inhibitor and a WEE1 inhibitor, wherein the combination of the AKT inhibitor and the WEE1 inhibitor is administered together as a combination formulation or separately.

According to aspects of the present invention, methods of treating cancer in a subject in need thereof are provided which include administering a combination of an AKT inhibitor and a WEE1 inhibitor, wherein the combination of the AKT inhibitor and the WEE1 inhibitor is administered together as a combination formulation or separately and wherein the methods exclude administration of CHK1 inhibitors, mTOR inhibitors or both CHK1 inhibitors and mTOR inhibitors.

Methods of treating cancer in a subject in need thereof are provided according to aspects of the present invention which include administering a combination of an AKT inhibitor selected from the group consisting of: AZD5363, GDC0068, a combination of AZD5363 and GDC0068, a pharmaceutically acceptable salt, hydrate, amide or ester of any thereof, and an siRNA directed to AKT; and a WEE1 inhibitor, wherein the combination of the AKT inhibitor and the WEE1 inhibitor is administered together as a combination formulation or separately.

Methods of treating cancer in a subject in need thereof are provided according to aspects of the present invention which include administering a combination of an AKT inhibitor and a WEE1 inhibitor selected from the group consisting of: MK1775, a pharmaceutically acceptable salt, hydrate, amide or ester thereof and an siRNA directed to WEE1, wherein the combination of the AKT inhibitor and the WEE1 inhibitor is administered together as a combination formulation or separately.

Methods of treating cancer in a subject in need thereof are provided according to aspects of the present invention which include administering a combination of an AKT inhibitor selected from the group consisting of: AZD5363, GDC0068, a combination of AZD5363 and GDC0068, a pharmaceutically acceptable salt, hydrate, amide or ester of any thereof, and an siRNA directed to AKT; and a WEE1 inhibitor selected from the group consisting of: MK1775, a pharmaceutically acceptable salt, hydrate, amide or ester thereof and an siRNA directed to WEE1, wherein the combination of the AKT inhibitor and the WEE1 inhibitor is administered together as a combination formulation or separately.

Methods of treating cancer in a subject in need thereof are provided according to aspects of the present invention which include administering a combination of an AKT inhibitor and a WEE1 inhibitor, wherein the combination of the AKT inhibitor and the WEE1 inhibitor is administered together as a combination formulation or separately, wherein administration of the combination provides a synergistic effect.

Methods of treating cancer in a subject in need thereof are provided according to aspects of the present invention, wherein the cancer is characterized by constitutive activation of a mitogen-activated protein kinase-signaling pathway, and which include administering a combination of an AKT inhibitor and a WEE1 inhibitor, wherein the combination of the AKT inhibitor and the WEE1 inhibitor is administered together as a combination formulation or separately.

Methods of treating cancer in a subject in need thereof are provided according to aspects of the present invention, wherein the cancer is characterized by constitutive activation of a mitogen-activated protein kinase-signaling pathway associated with one or more mutations in BRAF, KIT and/or RAS, and which include administering a combination of an AKT inhibitor and a WEE1 inhibitor, wherein the combination of the AKT inhibitor and the WEE1 inhibitor is administered together as a combination formulation or separately.

Methods of treating cancer in a subject in need thereof are provided according to aspects of the present invention, wherein the cancer is characterized by constitutive activation of a mitogen-activated protein kinase-signaling pathway associated with V600E BRAF, and which include administering a combination of an AKT inhibitor and a WEE1 inhibitor, wherein the combination of the AKT inhibitor and the WEE1 inhibitor is administered together as a combination formulation or separately.

Methods of treating cancer in a subject in need thereof are provided according to aspects of the present invention, wherein the cancer is characterized by AKT dysregulation, and which include administering a combination of an AKT inhibitor and a WEE1 inhibitor, wherein the combination of the AKT inhibitor and the WEE1 inhibitor is administered together as a combination formulation or separately.

Methods of treating cancer in a subject in need thereof are provided according to aspects of the present invention, wherein the cancer is melanoma, colorectal cancer, thyroid cancer, breast cancer, prostate cancer, sarcoma, glioblastoma, T-cell acute lymphoblastic leukaemia, lung cancer or liver cancer, and which include administering a combination of an AKT inhibitor and a WEE1 inhibitor, wherein the combination of the AKT inhibitor and the WEE1 inhibitor is administered together as a combination formulation or separately.

Methods of treating cancer in a subject in need thereof are provided according to aspects of the present invention which include obtaining a first sample containing or suspected of containing cancer cells from the subject prior to administering the combination of the AKT inhibitor and the WEE1 inhibitor; administering a combination of an AKT inhibitor and a WEE1 inhibitor, wherein the combination of the AKT inhibitor and the WEE1 inhibitor is administered together as a combination formulation or separately, obtaining a second sample containing or suspected of containing cancer cells from the subject after administering the combination of the AKT inhibitor and the WEE1 inhibitor; and assaying the first and second samples for one or more markers of apoptosis, thereby monitoring effectiveness of administering the combination of the AKT inhibitor and the WEE1 inhibitor.

Methods of treating cancer in a subject in need thereof are provided according to aspects of the present invention which include obtaining a first sample containing or suspected of containing cancer cells from the subject prior to administering the combination of the AKT inhibitor and the WEE1 inhibitor; administering a combination of an AKT inhibitor and a WEE1 inhibitor, wherein the combination of the AKT inhibitor and the WEE1 inhibitor is administered together as a combination formulation or separately, obtaining a second sample containing or suspected of containing cancer cells from the subject after administering the combination of the AKT inhibitor and the WEE1 inhibitor; and assaying the first and second samples for activity of a mitogen-activated protein kinase-signaling pathway, thereby monitoring effectiveness of administering the combination of the AKT inhibitor and the WEE1 inhibitor.

Methods of treating cancer in a subject in need thereof are provided according to aspects of the present invention which include obtaining a first sample containing or suspected of containing cancer cells from the subject prior to administering the combination of the AKT inhibitor and the WEE1 inhibitor; administering a combination of an AKT inhibitor and a WEE1 inhibitor, wherein the combination of the AKT inhibitor and the WEE1 inhibitor is administered together as a combination formulation or separately, obtaining a second sample containing or suspected of containing cancer cells from the subject after administering the combination of the AKT inhibitor and the WEE1 inhibitor; and assaying the first and second samples for AKT dysregulation, thereby monitoring effectiveness of administering the combination of the AKT inhibitor and the WEE1 inhibitor.

Methods of treating cancer in a subject in need thereof are provided according to aspects of the present invention which include obtaining a first sample containing or suspected of containing cancer cells from the subject prior to administering the combination of the AKT inhibitor and the WEE1 inhibitor; administering a combination of an AKT inhibitor and a WEE1 inhibitor, wherein the combination of the AKT inhibitor and the WEE1 inhibitor is administered together as a combination formulation or separately, obtaining a second sample containing or suspected of containing cancer cells from the subject after administering the combination of the AKT inhibitor and the WEE1 inhibitor; and assaying the first and second samples for p53 expression and/or an associated gene selected from the group consisting of: CLCA2, PVRL4, SULF2, CDKN a, BTG2, ACTA2, TP53, FDXR, GDF15, IGFBP5 and ADAM19, wherein an increase in p53 expression and/or expression of an associated gene selected from the group consisting of: CLCA2, PVRL4, SULF2, CDKN a, BTG2, ACTA2, TP53, FDXR, GDF15, IGFBP5 and ADAM19, is an indicator of an anti-cancer cell effect of treatment with the combination of the AKT inhibitor and the WEE1 inhibitor, thereby monitoring effectiveness of administering the combination of the AKT inhibitor and the WEE1 inhibitor

Methods of treating cancer in a subject in need thereof are provided according to aspects of the present invention which include obtaining a first sample containing or suspected of containing cancer cells from the subject prior to administering the combination of the AKT inhibitor and the WEE1 inhibitor; administering a combination of an AKT inhibitor and a WEE1 inhibitor, wherein the combination of the AKT inhibitor and the WEE1 inhibitor is administered together as a combination formulation or separately, obtaining a second sample containing or suspected of containing cancer cells from the subject after administering the combination of the AKT inhibitor and the WEE1 inhibitor; and assaying the first and second samples for FOXM1 expression and/or a expression of an associated gene selected from the group consisting of: TMPO, ANP32E, SMC4, KIF20B, ASPM, DEPDC1, NCAPG, CENPE, wherein a decrease in expression of FOXM1 and/or an associated gene selected from the group consisting of: TMPO, ANP32E, SMC4, KIF20B, ASPM, DEPDC1, NCAPG and CENPE, is an indicator of an anti-cancer cell effect of treatment with the combination of the AKT inhibitor and the WEE1 inhibitor, thereby monitoring effectiveness of administering the combination of the AKT inhibitor and the WEE1 inhibitor.

Methods of treating cancer in a subject in need thereof are provided according to aspects of the present invention which include administering a combination of an AKT inhibitor and a WEE1 inhibitor, wherein the AKT inhibitor and the WEE1 inhibitor are administered sequentially within a period of time selected from: one hour, two hours, four hours, eight hours, twelve hours, twenty-four hours, 2 days, 3 days, 4 days, 5 days, 6 days and 7 days.

Methods of treating cancer in a subject in need thereof are provided according to aspects of the present invention which include administering a combination of an AKT inhibitor and a WEE1 inhibitor, wherein the AKT inhibitor is an AKT3 inhibitor.

### BRIEF DESCRIPTION OF THE DRAWINGS

FIG. 1A is a graph showing the effect of siRNA targeting AKT3 (siAKT3), WEE1 (siWEE1) or combinations of siAKT3 and siWEE1 by measurement of human UACC 903 melanoma cell viability after 3 days of growth in serum free medium;

FIG. 1B is a graph showing the effect of siRNA targeting AKT3 (siAKT3), WEE1 (siWEE1) or combinations of siAKT3 and siWEE1 by measurement of human 1205 Lu melanoma cell viability after 3 days of growth in serum free medium;

FIG. 1C is a graph showing results of CalcuSyn analysis to calculate the combination index (CI) showed synergism between AKT3 and WEE1 when targeted together in human UACC 903 melanoma cells:

FIG. 1D is a graph showing results of CalcuSyn analysis to calculate the combination index (CI) showed synergism between AKT3 and WEE1 when targeted together in human 1205 Lu melanoma cells;

FIG. 1E is an image of Western blots showing siRNA-mediated knockdown of AKT3 and WEE1 protein levels in human UACC 903 melanoma cells;

FIG. 1F is an image of Western blots showing siRNA-mediated knockdown of AKT3 and WEE1 protein levels in human 1205 Lu melanoma cells;

FIG. 2A is a graph showing xenotransplant tumor volume over time in mice injected with 1205 Lu melanoma cells transfected with siScrambled control, anti-AKT3 siRNA (siAKT3), anti-WEE1 siRNA (siWEE1) or siAKT3 and siWEE1;

FIG. 2B is a graph showing percent inhibition of treatment (anti-WEE1 siRNA alone; anti-AKT3 siRNA alone; or anti-WEE1 siRNA and anti-AKT3 siRNA together) vs. control (scrambled siRNA) at day 21.5 from mice injected with 1205 Lu melanoma cells transfected with the indicated siRNA;

FIG. 2C is a graph showing xenotransplant tumor volume over time in mice injected with UACC 903 melanoma cells transfected with siScrambled control, anti-AKT3 siRNA (siAKT3), anti-WEE1 siRNA (siWEE1) or siAKT3 and siWEE1;

FIG. 2D is a graph showing percent inhibition of treatment (anti-WEE1 siRNA alone; anti-AKT3 siRNA alone; or anti-WEE1 siRNA and anti-AKT3 siRNA together) vs. control (scrambled siRNA) at day 21.5 from mice injected with UACC 9903 melanoma cells transfected with the indicated siRNA;

FIG. 2E is an image of Western blots showing siRNA-mediated knockdown of AKT3 or WEE1 protein expression in xenograft tumor lysates;

FIG. 2F is a graph showing analysis of proliferation of tumor cells in size and time matched tumors from mice injected with UACC 903 melanoma cells transfected with siScrambled controls or anti-AKT3 siRNA (siAKT3), anti-WEE1 siRNA (siWEE1) or siAKT3 and siWEE1;

FIG. 2G is a graph showing analysis of apoptosis in size and time matched tumors from mice injected with UACC 903 melanoma cells transfected with siScrambled controls or anti-AKT3 siRNA (siAKT3), anti-WEE1 siRNA (siWEE1) or siAKT3 and siWEE1;

FIG. 3A is a graph showing the effect of an AKT inhibitor (0.63, 1.25 or 2.5 micromolar GDC0068) alone, a WEE1 inhibitor (0.5 or 1.25 micromolar MK1775) alone or combinations of an AKT inhibitor and a WEE1 inhibitor (0.63, 1.25 or 2.5 micromolar GDC0068 and 0.5 or 1.25 micromolar MK1775), on UACC 903 human melanoma cancer cells;

FIG. 3B is a graph showing results of Chou-Talalay analysis for determining the combination index of the treatment of human UACC 903 human melanoma cancer cells with combinations of an AKT inhibitor and a WEE1 inhibitor (0.63 micromolar GDC0068 and 0.5 micromolar MK1775 shown as lightest circle, 1.25 micromolar GDC0068 and 0.5 micromolar MK1775, shown as medium intensity gray circle or 2.5 micromolar GDC0068 and 0.5 micromolar MK1775, shown as darkest gray circle; 0.63 micromolar GDC0068 and 1.25 micromolar MK1775 shown as lightest diamond, 1.25 micromolar GDC0068 and 1.25 micromolar MK1775, shown as medium intensity gray diamond or 2.5 micromolar GDC0068 and 1.25 micromolar MK1775, shown as darkest gray diamond, on UACC 903 human melanoma cancer cells;

FIG. 3C is a graph showing the effect of an AKT inhibitor (2.5, 5.0, 7.5 or 10 micromolar AZD5363) alone, a WEE1 inhibitor (0.63 MK1775) alone or combinations of an AKT inhibitor and a WEE1 inhibitor (2.5, 5.0. 7.5 or 10 micromolar AZD5363 and 0.63 micromolar MK1775), on UACC 903 human melanoma cancer cells;

FIG. 3D is a graph showing results of Chou-Talalay analysis for determining the combination index of the treatment of human UACC 903 human melanoma cancer cells with combinations of an AKT inhibitor and a WEE1 inhibitor (2.5 micromolar AZD5363 and 0.63 micromolar MK1775 shown as lightest circle, 5.0 micromolar AZD5363 and 0.63 micromolar MK1775, shown as medium intensity gray circle, 7.5 micromolar AZD5363 and 0.63 micromolar MK1775, shown as darkest gray circle or 10 micromolar AZD5363 and 0.63 micromolar MK1775, shown as black circle, on UACC 903 human melanoma cancer cells;

FIG. 3E is a graph showing the effect of an AKT inhibitor (0.31, 0.63, 1.25 or 2.5 micromolar GDC0068) alone, a WEE1 inhibitor (0.15 micromolar MK1775) alone or combinations of an AKT inhibitor and a WEE1 inhibitor (0.31, 0.63, 1.25 or 2.5 micromolar GDC0068 and 0.15 micromolar MK1775), on C8161.C19 melanoma cells;

FIG. 3F is a graph showing results of Chou-Talalay analysis for determining the combination index of the treatment of human UACC 903 human melanoma cancer cells with combinations of an AKT inhibitor and a WEE1 inhibitor (0.31 micromolar GDC0068 and 0.15 micromolar MK1775 shown as lightest circle, 0.63 micromolar GDC0068 and 0.15 micromolar MK1775, shown as medium intensity gray circle, 1.25 micromolar GDC0068 and 0.15 micromolar MK1775, shown as darkest gray circle or 2.5 micromolar GDC0068 and 0.15 micromolar MK1775, shown as black circle, on C8161.C19 melanoma cells:

FIG. 3G is a graph showing the effect of an AKT inhibitor (1.25, 2.5, 5.0 or 10 micromolar AZD5363) alone, a WEE1 inhibitor (0.15 micromolar MK1775) alone or combinations of an AKT inhibitor and a WEE1 inhibitor (2.5, 5.0. 7.5 or 10 micromolar AZD5363 and 0.15 micromolar MK1775), on C8161.C19 melanoma cells;

FIG. 3H is a graph showing results of Chou-Talalay analysis for determining the combination index of the treatment of human UACC 903 human melanoma cancer cells with combinations of an AKT inhibitor and a WEE1 inhibitor (1.25 micromolar AZD5363 and 0.15 micromolar MK1775 shown as lightest circle, 2.5 micromolar AZD5363 and 0.15 micromolar MK1775, shown as medium intensity gray circle, 5.0 micromolar AZD5363 and 0.15 micromolar MK1775, shown as darkest gray circle or 10 micromolar AZD5363 and 0.15 micromolar MK1775, shown as black circle, on C8161.C19 melanoma cells;

FIG. 4A is a set of graphs showing tumor kinetics of UACC 903 melanoma xenografts in mice following oral administration of vehicle, a WEE1 inhibitor (MK1775, 50 mg/kg), an AKT inhibitor (AZD5363, 150 mg/kg) or a combination of a MK1775, 50 mg/kg and AZD5363, 150 mg/kg, the inset graph shows the average weights of mice in each treatment group;

FIG. 4B is a graph showing sizes of tumors obtained from mice following the treatments described for FIG. 4A where error bars show standard error of the mean (SEM);

FIG. 4C is a graph showing tumor kinetics of 1205 Lu melanoma xenografts in mice following oral administration of vehicle, MK1775 (50 mg/kg), AZD5363 (150 mg/kg) or a combination of MK1775, 50 mg/kg and AZD5363, 150 mg/kg;

FIG. 4D is a graph showing the average weights of mice in each treatment group described for FIG. 4C;

FIG. 4E is a graph showing tumor kinetics of 1205 Lu melanoma xenografts in mice following oral administration, twice per day (b.i.d.), of vehicle, MK1775 (30 mg/kg, 3 days on and 4 days off); AZD5363 (130 mg/kg, 4 days on and 3 days off) or a combination of AZD5363, 130 mg/kg for first 4 days followed by MK1775, 30 mg/kg for next 3 days of the week;

FIG. 4F is a graph showing the average weights of mice in each treatment group described for FIG. 4E;

FIG. 5A is an image of Western blots showing the effect of siRNA targeting AKT3 (siAKT3) alone, siRNA targeting WEE1 (siWEE1) alone or siAKT3 in combination with increasing amounts of siWEE1 introduced into UACC 903 human melanoma cells via nucleofection, where protein lysates were collected and analyzed 2 days after nucleofection;

FIG. 5B is an image of Western blots showing the effect of siRNA targeting AKT3 (siAKT3) alone, siRNA targeting WEE1 (siWEE1) alone or siAKT3 in combination with increasing amounts of siWEE1 introduced into 1205 Lu human melanoma cells via nucleofection, where protein lysates were collected and analyzed 2 days after nucleofection;

FIG. 5C is an image of Western blots showing the changes in the levels Histone H2A.X, p53, p21, p27, pPRAS40, FOXM1, PLK, phosphorylation of CDK1 and serine phosphorylation of RBI proteins in UACC 903 melanoma cells treated with a WEE1 inhibitor (1 μM MK1775), an AKT inhibitor (1 μM, 3 μM or 10 μM AZD5363) or a combination of 1 μM MK1775 and 1 μM, 3 μM or 10 μM AZD5363 of AZD5363;

FIG. 5D is a diagram showing the mechanism of synergism for co-targeting AKT and WEE1 signaling pathways;

FIG. 6A is a graph showing the effect of an AKT inhibitor (5, 10 or 20 micromolar AZD5363) alone, a WEE1 inhibitor (0.63 micromolar MK1775) alone or combinations of an AKT inhibitor and a WEE1 inhibitor (5 micromolar AZD5363 and 0.63 micromolar MK1775, 10 micromolar AZD5363 and 0.63 micromolar MK1775 or 15 micromolar AZD5363 and 0.63 micromolar MK1775) on human MCF-7 breast cancer cells;

FIG. 6B is a graph showing results of Chou-Talalay analysis for determining the combination index of the treatment of human MCF-7 breast cancer cells with combinations of an AKT inhibitor and a WEE1 inhibitor (5 micromolar AZD5363 and 0.63 micromolar MK1775 shown as lightest circle, 10 micromolar AZD5363 and 0.63 micromolar MK1775, shown as medium intensity gray circle, on human MCF-7 breast cancer cells;

FIG. 6C is a graph showing the effect of an AKT inhibitor (5, 10 or 20 micromolar AZD5363) alone, a WEE1 inhibitor (0.63 micromolar MK1775) alone or combinations of an AKT inhibitor and a WEE1 inhibitor (5 micromolar AZD5363 and 0.63 micromolar MK1775, 10 micromolar AZD5363 and 0.63 micromolar MK1775 or 15 micromolar AZD5363 and 0.63 micromolar MK1775) on human PC-3 prostate cancer cells;

FIG. 6D is a graph showing results of Chou-Talalay analysis for determining the combination index of the treatment of human MCF-7 breast cancer cells with combinations of an AKT inhibitor and a WEE1 inhibitor (5 micromolar AZD5363 and 0.63 micromolar MK1775 shown as lightest circle, 10 micromolar AZD5363 and 0.63 micromolar MK1775, shown as medium intensity gray circle or 15 micromolar AZD5363 and 0.63 micromolar MK1775, shown as darkest gray circle, on human PC-3 prostate cancer cells;

FIG. 7A is an image showing a section of heart tissue obtained from a mouse treated with vehicle (control);

FIG. 7B is an image showing a section of heart tissue obtained from a mouse treated with AZD5363;

FIG. 7C is an image showing a section of heart tissue obtained from a mouse treated with MK1775;

FIG. 7D is an image showing a section of heart tissue obtained from a mouse treated with a combination of AZD5363 and MK1775;

FIG. 7E is an image showing a section of lungs tissue obtained from a mouse treated with vehicle (control);

FIG. 7F is an image showing a section of lungs tissue obtained from a mouse treated with AZD5363;

FIG. 7G is an image showing a section of lungs tissue obtained from a mouse treated with MK1775;

FIG. 7H is an image showing a section of lungs tissue obtained from a mouse treated with a combination of AZD5363 and MK1775;

FIG. 7I is an image showing a section of liver tissue obtained from a mouse treated with vehicle (control);

FIG. 7J is an image showing a section of liver tissue obtained from a mouse treated with AZD5363;

FIG. 7K is an image showing a section of liver tissue obtained from a mouse treated with MK1775;

FIG. 7L is an image showing a section of liver tissue obtained from a mouse treated with a combination of AZD5363 and MK1775;

FIG. 7M is an image showing a section of kidney tissue obtained from a mouse treated with vehicle (control);

FIG. 7N is an image showing a section of kidney tissue obtained from a mouse treated with AZD5363;

FIG. 7O is an image showing a section of kidney tissue obtained from a mouse treated with MK1775;

FIG. 7P is an image showing a section of kidney tissue obtained from a mouse treated with a combination of AZD5363 and MK1775;

FIG. 7Q is an image showing a section of spleen tissue obtained from a mouse treated with vehicle (control);

FIG. 7R is an image showing a section of spleen tissue obtained from a mouse treated with AZD5363;

FIG. 7S is an image showing a section of spleen tissue obtained from a mouse treated with MK1775;

FIG. 7T is an image showing a section of spleen tissue obtained from a mouse treated with a combination of AZD5363 and MK1775;

FIG. 7U is an image showing a section of intestine tissue obtained from a mouse treated with vehicle (control);

FIG. 7V is an image showing a section of intestine tissue obtained from a mouse treated with AZD5363;

FIG. 7W is an image showing a section of intestine tissue obtained from a mouse treated with MK1775;

FIG. 7X is an image showing a section of intestine tissue obtained from a mouse treated with a combination of AZD5363 and MK1775;

FIG. 8 shows a Western blot showing MK1775 or GDC0068 induced alterations in levels of Histone H2A.X, p53, CHK1, p21, p27, phosphorylation of CDK1 and AKT, serine phosphorylation of RBI proteins, phosphorylation of H2AX and CHK1;

FIG. 9A is a Western blot image showing p53 expression in lysates from day 11 UACC 903 xenograft tumor following oral administration of AZD5363, MK1775 or a combination of AZD5363 and MK1775 to tumor-bearing mice; and

FIG. 9B is a bar graph showing quantification of p53 expression in lysates from day 11 UACC 903 xenograft tumor following oral administration of AZD5363, MK1775 or a combination of AZD5363 and MK1775 to tumor-bearing mice.

### DETAILED DESCRIPTION OF THE INVENTION

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols: 5th Ed., 2002: B. Alberts et al., Molecular Biology of the Cell, 4th Ed., Garland, 2002; D. L. Nelson and M. M. Cox, Lehninger Principles of Biochemistry, 4th Ed., W.H. Freeman & Company, 2004; Engelke, D. R., RNA Interference (RNAi): Nuts and Bolts of RNAi Technology, DNA Press LLC, Eagleville, P A, 2003; Herdewijn, P. (Ed.), Oligonucleotide Synthesis: Methods and Applications, Methods in Molecular Biology, Humana Press, 2004; Chu, E. and Devita, V. T., Eds., Physicians' Cancer Chemotherapy Drug Manual, Jones & Bartlett Publishers, 2005; J. M. Kirkwood et al., Eds., Current Cancer Therapeutics, 4th Ed., Current Medicine Group, 2001; Remington: The Science and Practice of Pharmacy, Lippincott Williams & Wilkins, 21st Ed., 2005; L. V. Allen, Jr. et al., Ansel's Pharmaceutical Dosage Forms and Drug Delivery Systems, 8th Ed., Philadelphia, Pa.: Lippincott, Williams & Wilkins, 2004; and L. Brunton et al., Goodman & Gilman's The Pharmacological Basis of Therapeutics, McGraw-Hill Professional, 12th Ed., 2011.

The singular terms “a,” “an,” and “the” are not intended to be limiting and include plural referents unless explicitly stated otherwise or the context clearly indicates otherwise.

Synergistic effects of combination compositions and treatments including administration of an AKT inhibitor and a WEE1 inhibitor are unexpectedly found as described herein.

AKT is a serine/threonine protein kinase, also known as protein kinase B, which has a stimulatory effect on cell cycle progression, cell proliferation and inhibition of apoptosis. AKT proteins, nucleic acids and signaling pathway components are described, for instance, see Testa, J. R. et al., PNAS, 98:10983-10985; Fayard, E. et al., J. Cell Sci., 118:5675-5678, 2005; Cheng, J. and S. Nicosia, (2001) AKT signal transduction pathway in oncogenesis, in Encyclopedic Reference of Cancer, D. Schwab, Editor. 2001, Springer: Berlin, Germany, p. 35-7; Datta, S. R., et al. (1999) Cellular survival: a play in three AKTs. Genes Dev, 13(22): 2905-27; Fayard, E. et al. (2005) J Cell Sci, 118(Pt 24: 5675-8; Mirza, A. M., Fayard, E. et al. (2000) 2000. 11(6: 279-92; Nicholson, K. M. and N. G. Anderson, (2002)Cell Signal, 2002, 14(5): p. 381-95; Paez, J. and W. Sellers, (2003) PI3K/PTEN/AKT Pathway: A Critical Mediator of Oncogenic Signaling, in Signal Transduction in Cancer, D. Frank, Editor. 2003, Kluwer Academic Publishers: Netherlands; and Testa, J. R.; P. N. Tsichlis, (2005) Oncogene, 24(50): 7391-3 and other references listed herein.

AKT family members, AKT1, AKT2 and AKT3, are activated by phosphorylation, membrane translocation, increases in gene copy number and/or loss of a negative regulatory phosphatase, PTEN. Increased activation of AKT, including increased levels of AKT and/or increased levels of phosphorylated AKT is an indicator of AKT dysregulation associated with proliferation and cell survival in pathogenic conditions, such as cancer.

AKT 3 is active in ˜70% of melanomas. While all three AKT isoforms are expressed in melanocytes and melanoma cells, AKT3 is the predominantly active family member. Dysregulated AKT3 activity in melanoma cells reduces cellular apoptosis mediated through caspase-3, thereby promoting melanoma tumor development. As a well-established survival factor, hyperactivation of the AKT pathway is observed in many types of cancers, as described in Altomare et al, Oncogene 2005; 24(50): 7455-64.

AKT dysregulation is determined, for instance, by measurement of AKT gene copy number, AKT protein or RNA levels and/or levels of phosphorylated AKT, in cells known or suspected to be dysplastic, pre-cancerous, cancerous, metastatic or otherwise characterized by abnormal cell proliferation compared to normal cells. Assays for AKT dysregulation include, but are not limited to, immunoassays and nucleic acid assays.

The term “AKT inhibitor” refers to a substance having activity to specifically inhibit AKT activity in a cell in vitro or in vivo. Inhibition of AKT activity includes inhibition of all or one or more of AKT1, AKT2 and AKT3. An AKT inhibitor inhibits AKT activity abnormally activated in melanoma and other cancers and inhibits cancer cell survival and proliferation. An AKT inhibitor can be an agent effective to reduce the expression of all or one or more of AKT1, AKT2 and AKT3 in a cell, thereby inhibiting AKT activity. An AKT inhibitor reduces AKT activity in a cell by at least 10%, 20%, 30%, 40%, 50%, 60%, 70%, 80%, 90%, 95%, 99% or more, compared to a control.

The term “expression,” and grammatical equivalents, refers to transcription of a gene to produce a corresponding mRNA and/or translation of the mRNA to produce the corresponding protein.

According to aspects of the present invention, inhibition of AKT activity includes inhibition one or more of human AKT1, AKT2 and AKT3.

According to aspects of the present invention, the AKT inhibitor is an AKT3 inhibitor.

According to aspects of the present invention, the AKT inhibitor is an inhibitor of human AKT3.

The term “WEE1” as used herein refers to a protein kinase, particularly a human protein kinase. WEE1 is involved in the regulation of cell cycle by phosphorylating and inactivating cyclin-dependent kinase-1 (CDK1), see Watanabe et al., 1995, EMBO, 14:1878-1891.

As a component of G2/M checkpoint, WEE1 determines the time point for entry into mitosis and inhibits early progression of cell cycle. WEE1 is also involved in the coordination of cellular response to DNA damage. Furthermore, WEE1 was also identified as a key signaling molecule lying downstream of ^{V600E}BRAF in the MAPK signaling cascade, see Sharma et al., 2013, Am. J. Pathol., 182:1151-1162.

The term “WEE1 inhibitor” refers to a substance having activity to specifically inhibit WEE1 activity in a cell in vitro or in vivo. Inhibition of WEE1 activity includes inhibition one or both of WEE1A and WEE1B. A WEE1 inhibitor inhibits WEE1 activity abnormally activated in melanoma and other cancers and inhibits cancer cell survival and proliferation. A WEE1 inhibitor can be an agent effective to reduce the expression of all or one or both of WEE1A and WEE1B in a cell, thereby inhibiting WEE1 activity. A WEE1 inhibitor reduces WEE1 activity in a cell by at least 10%, 20%, 30%, 40%, 50%, 60%, 70%, 80%, 90%, 95%, 99% or more, compared to a control.

According to aspects of the present invention, inhibition of WEE1 activity includes inhibition one or both of human WEE1A and WEE1B.

According to aspects of the present invention, the WEE1 inhibitor is a WEE1A inhibitor.

WEE1 inhibitors include, but are not limited to, MK1775 (commercially available from suppliers such as Chemie Tek, Indianapolis, Ind.), also known as MK-1775, AZD1775 and 2-allyl-1-(6-(2-hydroxypropan-2-yl)pyridin-2-yl)-6-((4-(4-methylpiperazin-1-yl)phenyl)amino)-1H-pyrazolo[3,4-d]pyrimidin-3(2H)-one; MK-3652; PD-166285; PF-00120130; anti-WEE1 antibodies; anti-WEE1 peptides; and anti-WEE1 nucleic acids such as anti-WEE1 siRNA, all of which can be obtained commercially or chemically synthesized according to known methods.

AKT inhibitors include, but are not limited to, GDC0068 (commercially available from suppliers such as Chemie Tek, Indianapolis, Ind.) also known as GDC-0068, ipatasertib and RG7440; MK-2206.2HCI; perifosine (also known as KRX-0401); GSK690693; AT7867; triciribine; CCT128930; A-674563; PHT-427; Akti-1/2; afuresertib (also known as GSK2110183); AT13148; GSK2141795; BAY1125976; uprosertib (aka GSK2141795); AZD5363 also known as AZD-5363 (commercially available from suppliers such as Chemie Tek, Indianapolis, Ind.); anti-AKT antibodies; anti-AKT peptides; and anti-AKT nucleic acids such as anti-AKT siRNA, all of which can be obtained commercially or chemically synthesized according to known methods.

WEE1 inhibitors include siRNA directed to WEE1 effective to decrease WEE1 protein in a cell containing the siRNA directed to WEE1.

According to aspects of the present invention, an siRNA directed to WEE1 is directed to WEE1A.

AKT inhibitors include siRNA directed to AKT effective to decrease AKT protein in a cell containing the siRNA directed to AKT.

According to aspects of the present invention, an siRNA directed to AKT is directed to AKT3.

An AKT inhibitor or WEE1 inhibitor can be an antibody.

As used herein, the terms “antibody” and “antibodies” relate to monoclonal antibodies, polyclonal antibodies, bispecific antibodies, multispecific antibodies, human antibodies, humanized antibodies, chimeric antibodies, camelized antibodies, single domain antibodies, single-chain Fvs (scFv), single chain antibodies, disulfide-linked Fvs (sdFv), and anti-idiotypic (anti-Id) antibodies (including, e.g., anti-Id antibodies to antibodies of the invention), and epitope-binding fragments of any of the above. In particular, antibodies include immunoglobulin molecules and immunologically active fragments of immunoglobulin molecules, i.e., molecules that contain an antigen binding site. Immunoglobulin molecules are of any type (e.g., IgG, IgE, IgM, IgD, IgA and IgY), class (e.g., IgG1, IgG2a, IgG2b, IgG2, IgG3, IgG4, IgA1, and IgA2), or subclass.

Examples of antibody fragments that can be an AKT inhibitor or WEE1 inhibitor further include Fab fragments, Fab′ fragments, F(ab′)2 fragments, Fd fragments, Fv fragments, scFv fragments, and domain antibodies (dAb). Antibody fragments may be generated by any technique known to one of skill in the art. For example, Fab and F(ab′)2 fragments may be produced by proteolytic cleavage of immunoglobulin molecules, using enzymes such as papain (to produce Fab fragments) or pepsin (to produce F(ab′) 2 fragments). F(ab′) 2 fragments contain the complete light chain, and the variable region, the CH 1 region and the hinge region of the heavy chain. Antibody fragments are also produced by recombinant DNA technologies. Antibody fragments may be one or more complementarity determining regions (CDRs) of antibodies.

An antibody inhibitor of AKT or WEE1 can be obtained commercially, isolated from an immunized animal or a monoclonal-producing hybridoma, or generated synthetically, such as by recombinant protein expression techniques.

Antibodies and methods for preparation of antibodies are well-known in the art. Details of methods of antibody generation and screening of generated antibodies for substantially specific binding to an antigen are described in standard references such as E. Harlow and D. Lane, Antibodies: A Laboratory Manual, Cold Spring Harbor Laboratory Press, 1988; F. Breitling and S. Dübel, Recombinant Antibodies, John Wiley & Sons, New York, 1999; H. Zola, Monoclonal Antibodies: Preparation and Use of Monoclonal Antibodies and Engineered Antibody Derivatives, Basics: From Background to Bench, BIOS Scientific Publishers, 2000; and B. K. C. Lo, Antibody Engineering: Methods and Protocols, Methods in Molecular Biology, Humana Press, 2003.

An AKT inhibitor or WEE1 inhibitor can be an anti-AKT or anti-WEE1 nucleic acid, such an antisense nucleic acid or an RNA interference nucleic acid, such as an siRNA. The term “siRNA” refers to a “small interfering RNA,” also known as a “short interfering RNA” which is a synthetic double stranded RNA which targets a specific mRNA for degradation, reducing or preventing translation of the mRNA in a cell. An siRNA is generally 15 to 40 base pairs in length, preferably 19 to 25 base pairs in length, and may be blunt ended or include a 3′ and/or 5′ overhang on each strand, wherein the overhang on each strand is independently 1, 2, 3, 4 or 5 nucleotides.

siRNA can be designed to specifically target to AKT or WEE1, as exemplified by siRNA disclosed herein and included in compositions and methods according to aspects of the present invention. An siRNA AKT inhibitor or siRNA WEE1 inhibitor can be obtained commercially or synthesized, see for example, Engelke, D. R., RNA Interference (RNAi): Nuts and Bolts of RNAi Technology, DNA Press LLC, Eagleville, Pa., 2003; and Herdewijn, P. (Ed.), Oligonucleotide Synthesis: Methods and Applications, Methods in Molecular Biology, Humana Press, 2004.

GDC0068 has the structural formula:

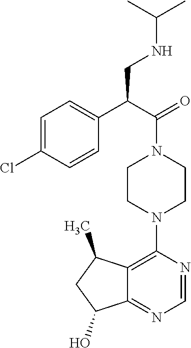 [SMILES: CC(C)NC[C@@H](C(=O)N1CCN(c2ncnc3c2[C@H](C)C[C@H]3O)CC1)c1ccc(Cl)cc1]

AZD5363 has the structural formula:

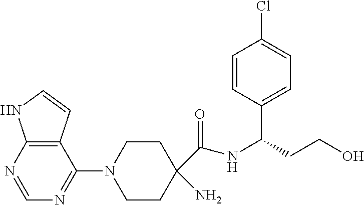 [SMILES: NC1(C(=O)N[C@@H](CCO)c2ccc(Cl)cc2)CCN(c2ncnc3[nH]ccc23)CC1]

MK1775 has the structural formula:

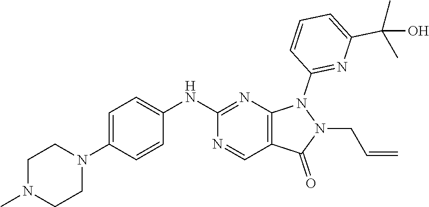 [SMILES: C=CCn1c(=O)c2cnc(Nc3ccc(N4CCN(C)CC4)cc3)nc2n1-c1cccc(C(C)(C)O)n1]

Compositions and pharmaceutical compositions including a WEE1 inhibitor encompass a pharmaceutically acceptable salt, hydrate, amide or ester of the WEE1 inhibitor according to aspects of the present invention. Compositions and pharmaceutical compositions including an AKT inhibitor may be provided as a pharmaceutically acceptable salt, hydrate, amide or ester of the AKT inhibitor according to aspects of the present invention.

Compositions and pharmaceutical compositions including MK1775 encompass a pharmaceutically acceptable salt, hydrate, amide or ester of MK1775 according to aspects of the present invention.

Compositions and pharmaceutical compositions including GDC0068 encompass a pharmaceutically acceptable salt, hydrate, amide or ester of GDC0068 according to aspects of the present invention.

Compositions and pharmaceutical compositions including AZD5363 encompass a pharmaceutically acceptable salt, hydrate, amide or ester of AZD5363 according to aspects of the present invention.

Compositions and pharmaceutical compositions according to the present invention encompass stereoisomers of a WEE1 inhibitor. Compositions and pharmaceutical compositions according to the present invention encompass the individual enantiomers of a WEE1 inhibitor as well as wholly or partially racemic mixtures of any of these.

Compositions and pharmaceutical compositions according to the present invention encompass stereoisomers of an AKT inhibitor. Compositions and pharmaceutical compositions according to the present invention encompass the individual enantiomers of an AKT inhibitor as well as wholly or partially racemic mixtures of any of these.

Compositions and pharmaceutical compositions according to the present invention encompass stereoisomers of MK1775. Compositions and pharmaceutical compositions according to the present invention encompass the individual enantiomers of MK1775, as well as wholly or partially racemic mixtures of any of these.

Compositions and pharmaceutical compositions according to the present invention encompass stereoisomers of GDC0068. Compositions and pharmaceutical compositions according to the present invention encompass the individual enantiomers of GDC0068, as well as wholly or partially racemic mixtures of any of these.

Compositions and pharmaceutical compositions according to the present invention encompass stereoisomers of AZD5363. Compositions and pharmaceutical compositions according to the present invention encompass the individual enantiomers of AZD5363, as well as wholly or partially racemic mixtures of any of these.

A derivative of an AKT inhibitor and a WEE1 inhibitor is optionally included in a composition or method according to aspects of the present invention. The term “derivative” as used herein refers to a compound that is modified compared to a reference compound and which has similar or improved bioactivity compared to the reference compound.

According to aspects, compositions and methods include an AKT inhibitor and a WEE1 inhibitor and exclude CHK1 inhibitors and mTOR inhibitors.

According to aspects, an AKT inhibitor included in compositions and methods of the present invention specifically inhibits AKT, i.e. all of AKT1, AKT2 and AKT3 or one or more of AKT1, AKT2 and AKT3, but does not substantially inhibit non-AKT protein kinases.

According to aspects, a WEE1 inhibitor included in compositions and methods of the present invention specifically inhibits WEE1, i.e. both of WEE1A and WEE1B or one of WEE1A and WEE1B, but does not substantially inhibit non-WEE1 protein kinases.

The term “pharmaceutically acceptable salt” refers to salts which are suitable for use in a subject without undue toxicity or irritation to the subject and which are effective for their intended use.

Pharmaceutically acceptable salts include pharmaceutically acceptable acid addition salts and base addition salts. Pharmaceutically acceptable salts are well-known in the art, such as those detailed in S. M. Berge et al., J. Pharm. Sci., 66:1-19, 1977. Exemplary pharmaceutically acceptable salts are those suitable for use in a subject without undue toxicity or irritation to the subject and which are effective for their intended use which are formed with inorganic acids such as hydrochloric acid, hydrobromic acid, hydroiodic acid, nitric acid, phosphoric acid, sulfuric acid and sulfamic acid; organic acids such as acetic acid, adipic acid, alginic acid, ascorbic acid, aspartic acid, benzenesulfonic acid, benzoic acid, 2-acetoxybenzoic acid, butyric acid, camphoric acid, camphorsulfonic acid, cinnamic acid, citric acid, digluconic acid, ethanesulfonic acid, formic acid, fumaric acid, glutamic acid, glycolic acid, glycerophosphoric acid, hemisulfic acid, heptanoic acid, hexanoic acid, 2-hydroxyethanesulfonic acid (isethionic acid), lactic acid, maleic acid, hydroxymaleic acid, malic acid, malonic acid, mandelic acid, mesitylenesulfonic acid, methanesulfonic acid, naphthalenesulfonic acid, nicotinic acid, 2-naphthalenesulfonic acid, oxalic acid, pamoic acid, pectinic acid, phenylacetic acid, 3-phenylpropionic acid, picric acid, pivalic acid, propionic acid, pyruvic acid, pyruvic acid, salicylic acid, stearic acid, succinic acid, sulfanilic acid, tartaric acid, p-toluenesulfonic acid, trichloroacetic acid, trifluoroacetic acid and undecanoic acid, inorganic bases such as ammonia, hydroxide, carbonate, and bicarbonate of ammonium; organic bases such as primary, secondary, tertiary and quaternary amine compounds ammonium, arginine, betaine, choline, caffeine, diolamine, diethylamine, diethanolamine, 2-dimethylaminoethanol, 2-diethylaminoethanol, dicyclohexylamine, dicyclohexylamine, dibenzylamine, N, N-dibenzylphenethylamine, 1-ephenamine, N, N′-dibenzylethylenediamine, ethanolamine, ethylamine, ethylenediamine, glucosamine, histidine, hydrabamine, isopropylamine, 1 h-imidazole, lysine, methylamine, N-ethylpiperidine, N-methylpiperidine, N-methylmorpholine, N, N-dimethylaniline, piperazine, trolamine, methylglucamine, purines, piperidine, pyridine, theobromine, tetramethylammonium compounds, tetraethylammonium compounds, trimethylamine, triethylamine, tripropylamine and tributylamine and metal cations such as aluminum, calcium, copper, iron, lithium, magnesium, manganese, potassium, sodium, and zinc.

Methods of treatment of a subject having, or at risk of having cancer are provided according to aspects of the present invention which include administering a combination of an AKT inhibitor and a WEE1 inhibitor, as a combination formulation or separately, wherein administration of the combination provides a synergistic effect.

Cancers treated using methods and compositions described herein are characterized by abnormal cell proliferation including, but not limited to, pre-neoplastic hyperproliferation, cancer in-situ, neoplasms and metastasis, and include solid and non-solid tumors. Examples of cancers treated according to aspects of the present invention include, but are not limited to, lymphoma, leukemia, squamous cell cancer, small-cell lung cancer, non-small cell lung cancer, adenocarcinoma of the lung, squamous carcinoma of the lung, cancer of the peritoneum, adrenal cancer, anal cancer, bile duct cancer, bladder cancer, brain cancer, breast cancer, triple negative breast cancer, central or peripheral nervous system cancers, cervical cancer, colon cancer, colorectal cancer, endometrial cancer, esophageal cancer, gall bladder cancer, gastrointestinal cancer, glioblastoma, head and neck cancer, kidney cancer, liver cancer, nasopharyngeal cancer, nasal cavity cancer, oropharyngeal cancer, oral cavity cancer, osteosarcoma, ovarian cancer, pancreatic cancer, parathyroid cancer, pituitary cancer, prostate cancer, retinoblastoma, sarcoma, salivary gland cancer, skin cancer, small intestine cancer, stomach cancer, testicular cancer, thymus cancer, thyroid cancer, uterine cancer, vaginal cancer and vulval cancer.

Cancers treated using methods and compositions according to aspects of the present invention are characterized by abnormal cell proliferation and AKT dysregulation.

Cancers treated using methods and compositions according to aspects of the present invention are characterized by high expression of AKT and/or WEE1 compared to normal cells. For example, prostate cancer treated using methods and compositions according to aspects of the present invention are characterized by high expression of AKT and/or WEE1 compared to normal prostate cells. Similarly, any cancer such as lymphoma, leukemia, squamous cell cancer, small-cell lung cancer, non-small cell lung cancer, adenocarcinoma of the lung, squamous carcinoma of the lung, cancer of the peritoneum, adrenal cancer, anal cancer, bile duct cancer, bladder cancer, brain cancer, breast cancer, triple negative breast cancer, central or peripheral nervous system cancers, cervical cancer, colon cancer, colorectal cancer, endometrial cancer, esophageal cancer, gall bladder cancer, gastrointestinal cancer, glioblastoma, head and neck cancer, kidney cancer, liver cancer, nasopharyngeal cancer, nasal cavity cancer, oropharyngeal cancer, oral cavity cancer, osteosarcoma, ovarian cancer, pancreatic cancer, parathyroid cancer, pituitary cancer, prostate cancer, retinoblastoma, sarcoma, salivary gland cancer, skin cancer, small intestine cancer, stomach cancer, testicular cancer, thymus cancer, thyroid cancer, uterine cancer, vaginal cancer and vulval cancer characterized by high expression of AKT and/or WEE1 compared to normal cells of a corresponding normal tissue are treated using methods and compositions according to aspects of the present invention by a method including administering a combination of an AKT inhibitor and a WEE1 inhibitor.

Methods of treatment of a subject having, or at risk of having cancer are provided according to aspects of the present invention which include administering a combination of an AKT inhibitor and a WEE1 inhibitor as a combination formulation or separately, wherein administration of the combination provides a synergistic effect.

Methods of treatment of a subject having, or at risk of having cancer are provided according to aspects of the present invention which include administering a combination of: 1) one or more of AZD5363, GDC0068, and an siRNA directed to AKT; and 2) one or both of MK1775 and an siRNA directed to WEE1; as a combination formulation or separately, wherein administration of the combination of 1) and 2) provides a synergistic effect.

Methods of treatment of a subject having, or at risk of having melanoma are provided according to aspects of the present invention which include administering a combination of an AKT inhibitor and a WEE1 inhibitor as a combination formulation or separately, wherein administration of the combination provides a synergistic effect.

Methods of treatment of a subject having, or at risk of having melanoma are provided according to aspects of the present invention which include administering a combination of: 1) one or more of AZD5363, GDC0068, and an siRNA directed to AKT: and 2) one or both of MK1775 and an siRNA directed to WEE1; as a combination formulation or separately, wherein administration of the combination of 1) and 2) provides a synergistic effect.

Methods and compositions of the present invention can be used for prophylaxis as well as amelioration of signs and/or symptoms of cancer. The terms “treating” and “treatment” used to refer to treatment of a cancer in a subject include: preventing, inhibiting or ameliorating the cancer in the subject, such as slowing progression of the cancer and/or reducing or ameliorating a sign or symptom of the cancer.

A therapeutically effective amount of an AKT inhibitor and a WEE1 inhibitor administered as a combination treatment of the present invention is an amount which has a beneficial effect in a subject being treated. In subjects having cancer or at risk for having cancer, such as a condition characterized by abnormal cell proliferation including, but not limited to, pre-neoplastic hyperproliferation, cancer in-situ, neoplasms, metastasis, a tumor, a benign growth or other condition responsive to a composition of the present invention, a therapeutically effective amount of a composition of the present invention is effective to ameliorate or prevent one or more signs and/or symptoms of the condition.

A subject treated according to methods and using compositions of the present invention can be mammalian or non-mammalian. A mammalian subject can be any mammal including, but not limited to, a human; a non-human primate; a rodent such as a mouse, rat, or guinea pig: a domesticated pet such as a cat or dog; a horse, cow, pig, sheep, goat, or rabbit. A non-mammalian subject can be any non-mammal including, but not limited to, a bird such as a duck, goose, chicken, or turkey. Subjects can be either gender and can be any age. In aspects of methods including administration of an inventive pharmaceutical composition to a subject, the subject is human. The terms “subject” and “patient” are used interchangeably herein.

Combinations of an AKT inhibitor, a WEE1 inhibitor, and one or more additional therapeutic agents are administered according to aspects of the present invention.

Combinations of: 1) one or more of AZD5363, GDC0068 and an siRNA directed to AKT; 2) one or both of MK1775 and an siRNA directed to WEE1; and 3) one or more additional therapeutic agents, are administered according to aspects of the present invention

The term “additional therapeutic agent” is used herein to refer to a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues which is a biologically, physiologically, or pharmacologically active substance (or substances) that acts locally or systemically in a subject.

Additional therapeutic agents included according to aspects of methods and compositions of the present invention include, but are not limited to, antibiotics, antivirals, antineoplastic agents, analgesics, antipyretics, antidepressants, antipsychotics, anti-cancer agents, antihistamines, anti-osteoporosis agents, anti-osteonecrosis agents, antiinflammatory agents, anxiolytics, chemotherapeutic agents, diuretics, growth factors, hormones, non-steroidal anti-inflammatory agents, steroids and vasoactive agents.

Combination therapies including administration of an AKT inhibitor and a WEE1 inhibitor show synergistic effects.

According to aspects of the present invention, combination therapies include: (A) pharmaceutical compositions that include a pharmaceutical combination composition including an AKT inhibitor and a WEE1 inhibitor formulated together in a single pharmaceutical composition; and/or (B) co-administration of an AKT inhibitor and a WEE1 inhibitor wherein the AKT inhibitor and the WEE1 inhibitor have not been formulated in the same composition. When using separate formulations the AKT inhibitor may be administered at the same time, intermittent times, staggered times, prior to, subsequent to, or combinations thereof, with reference to the WEE1 inhibitor.

According to aspects of the present invention, combination therapies include: (A) pharmaceutical compositions that include a pharmaceutical combination composition including: 1) one or more of AZD5363, GDC0068 and an siRNA directed to AKT; and 2) one or both of MK1775 and an siRNA directed to WEE1; formulated together in a single pharmaceutical composition; and/or (B) co-administration of 1) one or more of AZD5363, GDC0068 and an siRNA directed to AKT; and 2) one or both of MK1775 and an siRNA directed to WEE1; wherein the components 1) and 2), have not been formulated in the same composition. When using separate formulations, the component 1); may be administered at the same time, intermittent times, staggered times, prior to, subsequent to, or combinations thereof, with reference to the component 2).

According to aspects of the present invention, combination therapies include: (A) pharmaceutical compositions that include a pharmaceutical combination composition including an AKT inhibitor and a WEE1 inhibitor formulated together with one or more additional therapeutic agents in a single pharmaceutical composition; (B) co-administration of an AKT inhibitor, a WEE1 inhibitor, and one or more additional pharmaceutical agents wherein the AKT inhibitor, the WEE1 inhibitor and the one or more additional pharmaceutical agents have not been formulated in the same composition; and/or (C) co-administration of an AKT inhibitor, a WEE1 inhibitor and one or more additional pharmaceutical agents wherein two or more, but not all, of: the AKT inhibitor, the WEE1 inhibitor and the one or more additional pharmaceutical agents are formulated in the same composition. When using separate formulations each of the AKT inhibitor, the WEE1 inhibitor and one or more additional pharmaceutical agents may be administered at the same time, intermittent times, staggered times, prior to, subsequent to, or combinations thereof, with reference to each of the other components.

According to aspects of the present invention, combination therapies include: (A) pharmaceutical compositions that include a pharmaceutical combination composition including: 1) one or more of AZD5363, GDC0068 and an siRNA directed to AKT; and 2) one or both of MK1775 and an siRNA directed to WEE1; formulated together with 3) one or more additional therapeutic agents in a single pharmaceutical composition; (B) co-administration of 1) one or more of AZD5363, GDC0068 and an siRNA directed to AKT; 2) one or both of MK1775 and an siRNA directed to WEE1; and 3) one or more additional pharmaceutical agents, wherein the one or more of AZD5363, GDC0068 and an siRNA directed to AKT, the one or both of MK1775 and an siRNA directed to WEE1 and the one or more additional pharmaceutical agents have not been formulated in the same composition; and/or (C) co-administration of: 1) one or more of AZD5363, GDC0068 and an siRNA directed to AKT; 2) one or both of MK1775 and an siRNA directed to WEE1; and 3) one or more additional pharmaceutical agents, wherein two or more, but not all, of: the one or more of AZD5363, GDC0068 and an siRNA directed to AKT; one or both of MK1775 and an siRNA directed to WEE1; and the one or more additional pharmaceutical agents are formulated in the same composition. When using separate formulations, the one or more of AZD5363, GDC0068 and an siRNA directed to AKT; one or both of MK1775 and an siRNA directed to WEE1; and the one or more additional pharmaceutical agents may be administered at the same time, intermittent times, staggered times, prior to, subsequent to, or combinations thereof, with reference to each of the other components.

According to aspects of the present invention, the AKT inhibitor and the WEE1 inhibitor are administered together daily, administered together twice daily or administered together more often in one day.

According to aspects of the present invention, the AKT inhibitor and the WEE1 inhibitor are both administered separately daily, both administered separately twice daily or both administered separately more often in one day.

According to aspects of the present invention, the AKT inhibitor and the WEE1 inhibitor are administered sequentially within a period of time selected from: one hour, two hours, four hours, eight hours, twelve hours, twenty-four hours, 2 days, 3 days, 4 days, 5 days, 6 days or 1 week in a method of treatment of cancer in a subject.

According to aspects of the present invention, the AKT inhibitor is administered weekly, twice weekly, three times in a week, every other day, daily, administered twice daily or administered more often in one day and the WEE1 inhibitor is administered less often or more often than the AKT inhibitor in a treatment for cancer to achieve a synergistic effect of the combined administration to the subject.

According to aspects of the present invention, the AKT inhibitor and the WEE1 inhibitor are administered together or separately at the same time, intermittent times, or staggered times during a treatment period which can be from 1 day to 100 days, such as 1 day to 2 days, 2 day to 3 days, 3 day to 5 days, 5 day to 7 days, 7 days to 14 days, 14 days to 21 days, 21 days to 28 days, 28 days to 35 days, 35 days to 50 days or 50 days to 60 days and which may include one or more periods in which the amount of the AKT inhibitor and/or the WEE1 inhibitor is increased or decreased, in which the identity of the particular AKT inhibitor and/or the WEE1 inhibitor is changed, or in which no treatment is given.

Combination treatments can allow for reduced effective dosage and increased therapeutic index of the pharmaceutical composition including an AKT inhibitor and a WEE1 inhibitor.

An additional pharmaceutical agent is an anti-cancer agent according to aspects of the present invention.

Anti-cancer agents are described, for example, in Goodman et al., Goodman and Gilman's The Pharmacological Basis of Therapeutics, 8th Ed., Macmillan Publishing Co., 1990.

Anti-cancer agents illustratively include acivicin, aclarubicin, acodazole, acronine, adozelesin, aldesleukin, alitretinoin, allopurinol, altretamine, ambomycin, ametantrone, amifostine, aminoglutethimide, amsacrine, anastrozole, anthramycin, arsenic trioxide, asparaginase, asperlin, azacitidine, azetepa, azotomycin, batimastat, benzodepa, bevacizumab, bicalutamide, bisantrene, bisnafide dimesylate, bizelesin, bleomycin, brequinar, bropirimine, busulfan, cactinomycin, calusterone, capecitabine, caracemide, carbetimer, carboplatin, carmustine, carubicin, carzelesin, cedefingol, celecoxib, chlorambucil, cirolemycin, cisplatin, cladribine, cobimetinib, crisnatol mesylate, cyclophosphamide, cytarabine, dacarbazine, dactinomycin, daunorubicin, decitabine, dexormaplatin, dezaguanine, dezaguanine mesylate, diaziquone, docetaxel, doxorubicin, droloxifene, dromostanolone, duazomycin, edatrexate, eflomithine, elsamitrucin, enloplatin, enpromate, epipropidine, epirubicin, erbulozole, esorubicin, estramustine, etanidazole, etoposide, etoprine, fadrozole, fazarabine, fenretinide, floxuridine, fludarabine, fluorouracil, flurocitabine, fosquidone, fostriecin, fulvestrant, gemcitabine, hydroxyurea, idarubicin, ifosfamide, ilmofosine, interleukin II (IL-2, including recombinant interleukin II or rIL2), interferon alfa-2a, interferon alfa-2b, interferon alfa-n1, interferon alfa-n3, interferon beta-Ia, interferon gamma-Ib, iproplatin, irinotecan, lanreotide, letrozole, leuprolide, liarozole, lometrexol, lomustine, losoxantrone, masoprocol, maytansine, mechlorethamine hydrochlride, megestrol, melengestrol acetate, melphalan, menogaril, mercaptopurine, methotrexate, metoprine, meturedepa, mitindomide, mitocarcin, mitocromin, mitogillin, mitomalcin, mitomycin, mitosper, mitotane, mitoxantrone, mycophenolic acid, nelarabine, nocodazole, nogalamycin, ormnaplatin, oxisuran, paclitaxel, pegaspargase, peliomycin, pentamustine, peplomycin, perfosfamide, pipobroman, piposulfan, piroxantrone hydrochloride, plicamycin, plomestane, porfimer, porfiromycin, prednimustine, procarbazine, puromycin, pyrazofurin, riboprine, rogletimide, safingol, semustine, simtrazene, sparfosate, sparsomycin, spirogermanium, spiromustine, spiroplatin, streptonigrin, streptozocin, sulofenur, talisomycin, tamoxifen, tecogalan, tegafur, teloxantrone, temoporfin, teniposide, teroxirone, testolactone, thiamiprine, thioguanine, thiotepa, tiazofurin, tirapazamine, topotecan, toremifene, trestolone, triciribine, trimetrexate, triptorelin, tubulozole, uracil mustard, uredepa, vapreotide, vemurafenib, verteporfin, vinblastine, vincristine sulfate, vindesine, vinepidine, vinglycinate, vinleurosine, vinorelbine, vinrosidine, vinzolidine, vorozole, zeniplatin, zinostatin, zoledronate, and zorubicin.

According to aspects of the present invention, one or more correlative biomarkers of therapeutic activity of an AKT inhibitor and a WEE1 inhibitor administered as a combination treatment of the present invention to treat cancer in a subject in need thereof are assayed to assess treatment of the cancer in the subject. Biomarkers of apoptosis are correlative biomarkers of therapeutic activity of an AKT inhibitor and a WEE1 inhibitor administered as a combination treatment of the present invention to treat cancer in a subject in need thereof and an increase in one or more biomarkers of apoptosis in cancer cells is indicative of efficacy of an AKT inhibitor and a WEE1 inhibitor administered as a combination treatment of the present invention to treat cancer in a subject in need thereof. Biomarkers of apoptosis include, but are not limited to, detection of DNA fragmentation, characteristic morphological changes distinct from necrosis and activation of caspase-3. Biomarkers of apoptosis are measured according to standard methodologies, for example as described herein.

According to aspects of the present invention, assays for effects of combination treatment with an AKT inhibitor and a WEE1 inhibitor are used to monitor a subject. Thus, for example, a test sample is obtained from the subject before treatment according to a method of the present invention and at one or more times during and/or following treatment in order to assess effectiveness of the treatment. In a further example, a test sample is obtained from the subject at various times in order to assess the course or progress of disease or healing.

In particular aspects, one or more additional biomarkers are assayed in a test sample obtained from a subject to aid in monitoring treatment with a pharmaceutical composition of the present invention. For example, apoptosis of cancer cells is assayed in a test sample obtained from a subject to aid in monitoring treatment with a pharmaceutical composition of the present invention. In a further example, AKT and/or WEE1 expression and/or activity is assayed in a test sample obtained from the subject to aid in monitoring treatment with a pharmaceutical composition of the present invention.

Optionally, a method of treating cancer in a subject in need thereof further includes an adjunct anti-cancer treatment. An adjunct anti-cancer treatment can be a radiation treatment of a subject or an affected area of a subject's body.

The dosage of an AKT inhibitor, a WEE1 inhibitor and any optional additional therapeutic agent will vary based on factors such as, but not limited to, the route of administration; the age, health, sex, and weight of the subject to whom the composition is to be administered; the nature and extent of the subject's symptoms, if any, and the effect desired. Dosage may be adjusted depending on whether treatment is to be acute or continuing. One of skill in the art can determine a pharmaceutically effective amount in view of these and other considerations typical in medical practice.

In particular aspects of inventive methods, the amount of the AKT inhibitor and/or WEE1 inhibitor administered in a combination treatment is less than an amount of the AKT inhibitor or WEE1 inhibitor necessary to achieve a therapeutic effect if the subject is treated with with either agent alone. Thus, in particular aspects of the present invention, the amount of an AKT inhibitor administered in combination with a WEE1 inhibitor in a treatment of cancer in a subject is at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, or at least 90% or more, less than an amount of the AKT inhibitor necessary to achieve a therapeutic effect when administered without a combination treatment of the present invention.

In particular aspects of inventive methods, the amount of the AKT inhibitor and/or WEE1 inhibitor administered in a combination treatment is less than an amount of the AKT inhibitor or WEE1 inhibitor necessary to achieve a therapeutic effect if the subject is treated with with either agent alone. Thus, in particular aspects of the present invention, the amount of a WEE1 inhibitor administered in combination with an AKT inhibitor in a treatment of cancer in a subject is at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, or at least 90%0 or more, less than an amount of the WEE1 inhibitor necessary to achieve a therapeutic effect when administered without a combination treatment of the present invention.

In general it is contemplated that a daily dosage of an AKT inhibitor, a WEE1 inhibitor and any optional additional therapeutic agent is in the range of about 0.001 to 100 milligrams per kilogram of a subject's body weight. A daily dose may be administered as two or more divided doses to obtain the desired effect. A pharmaceutical composition including any one or more of: an AKT inhibitor, a WEE1 inhibitor and any optional additional therapeutic agent, may also be formulated for sustained release to obtain desired results.

In particular aspects of inventive methods, an AKT inhibitor is administered in doses of 0.1 mg/day to 1 g/day, such as 0.1 mg/day to 0.25 mg/day, 0.25 mg/day to 0.5 mg/day, 0.5 mg/day to 0.75 mg/day, 0.75 mg/day to 1 mg/day, 0.25 mg/day to 500 mg/day, 0.5 mg/day to 200 mg/day, 0.75 mg/day to 100 mg/day, 1 mg/day to 2 mg/day, such as 2 mg/day to 5 mg/day, 5 mg/day to 10 mg/day, 5 mg/day to 20 mg/day, 10 mg/day to 20 mg/day, 20 mg/day to 30 mg/day, 30 mg/day to 40 mg/day, 40 mg/day to 50 mg/day, 40 mg/day to 60 mg/day, 60 mg/day to 70 mg/day, 70 mg/day to 80 mg/day, 80 mg/day to 90 mg/day, 90 mg/day to 95 mg/day, 95 mg/day to 100 mg/day, 100 mg/day to 150 mg/day, 150 mg/day to 200 mg/day, 200 mg/day to 250 mg/day, 250 mg/day to 300 mg/day, 300 mg/day to 350 mg/day, 350 mg/day to 400 mg/day, 400 mg/day to 450 mg/day, 450 mg/day to 500 mg/day, 500 mg/day to 550 mg/day, 550 mg/day to 600 mg/day, 600 mg/day to 650 mg/day, 650 mg/day to 700 mg/day, 700 mg/day to 750 mg/day, 750 mg/day to 800 mg/day, 800 mg/day to 850 mg/day, 850 mg/day to 900 mg/day, 900 mg/day to 950 mg/day or 950 mg/day to 1 g/day in a combination treatment with a WEE1 inhibitor.

In particular aspects of inventive methods, a WEE1 inhibitor is administered in doses of 0.1 mg/day to 1 g/day, such as 0.1 mg/day to 0.25 mg/day, 0.25 mg/day to 0.5 mg/day, 0.5 mg/day to 0.75 mg/day, 0.75 mg/day to 1 mg/day, 0.25 mg/day to 500 mg/day, 0.5 mg/day to 200 mg/day, 0.75 mg/day to 100 mg/day, 1 mg/day to 2 mg/day, such as 2 mg/day to 5 mg/day, 5 mg/day to 10 mg/day, 5 mg/day to 20 mg/day, 10 mg/day to 20 mg/day, 20 mg/day to 30 mg/day, 30 mg/day to 40 mg/day, 40 mg/day to 50 mg/day, 40 mg/day to 60 mg/day, 60 mg/day to 70 mg/day, 70 mg/day to 80 mg/day, 80 mg/day to 90 mg/day, 90 mg/day to 95 mg/day, 95 mg/day to 100 mg/day, 100 mg/day to 150 mg/day, 150 mg/day to 200 mg/day, 200 mg/day to 250 mg/day, 250 mg/day to 300 mg/day, 300 mg/day to 350 mg/day, 350 mg/day to 400 mg/day, 400 mg/day to 450 mg/day, 450 mg/day to 500 mg/day, 500 mg/day to 550 mg/day, 550 mg/day to 600 mg/day, 600 mg/day to 650 mg/day, 650 mg/day to 700 mg/day, 700 mg/day to 750 mg/day, 750 mg/day to 800 mg/day, 800 mg/day to 850 mg/day, 850 mg/day to 900 mg/day, 900 mg/day to 950 mg/day or 950 mg/day to 1 g/day in a combination treatment with an AKT inhibitor.

In particular aspects of inventive methods, an AKT inhibitor and a WEE1 inhibitor are administered in a ratio (mole:mole) in the range of 0.1:100 to 100:0.1, such as 0.25:50, 0.5:25, 0.75:15, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 15:0.75, 25:0.5 or 50:0.25. According to further particular aspects of inventive methods, an AKT inhibitor and a WEE1 inhibitor are administered in a ratio (mole:mole) in the range of 1:1.25, 0.15:1, 0.31:1, 0.63:1, 1.25:1, 12:1, 128:1, 16:1, 2.5:1, 32:1, 64:1 or 1:2.5.

In particular aspects of inventive methods, the amount of the adjunct anti-cancer treatment and/or anti-cancer agent administered is less than an amount of the adjunct anti-cancer treatment and/or anti-cancer agent necessary to achieve a therapeutic effect if administered without a combination treatment of the present invention including administration of an AKT inhibitor and a WEE1 inhibitor. Thus, in particular aspects of the present invention, the amount of an anti-cancer treatment and/or agent administered is at least 5%, at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, or at least 90%, less than an amount of the adjunct anti-cancer treatment and/or agent necessary to achieve a therapeutic effect when administered without a combination treatment of the present invention including administration of an AKT inhibitor and a WEE1 inhibitor.

Methods of the present invention include administration of a pharmaceutical composition of the present invention by a route of administration including, but not limited to, oral, rectal, nasal, pulmonary, epidural, ocular, otic, intraarterial, intracardiac, intracerebroventricular, intradermal, intravenous, intramuscular, intraperitoneal, intraosseous, intrathecal, intravesical, subcutaneous, topical, transdermal, and transmucosal, such as by sublingual, buccal, vaginal, and inhalational, routes of administration.

Combination Pharmaceutical Compositions

A combination pharmaceutical composition including both an AKT inhibitor and a WEE1 inhibitor according to the invention generally includes about 0.1-99% of an AKT inhibitor, about 0.1-99% of a WEE1 inhibitor; and a pharmaceutically acceptable carrier.

A combination pharmaceutical composition including 1) one or more of: AZD5363, GDC0068 and an siRNA directed to AKT and 2) one or both of MK1775 and an siRNA directed to WEE1; according to the invention generally includes about 0.1-99% of component 1), about 0.1-99% of component 2) and a pharmaceutically acceptable carrier.

A pharmaceutical composition of the present invention may be in any dosage form suitable for administration to a subject, illustratively including solid, semi-solid and liquid dosage forms such as tablets, capsules, powders, granules, suppositories, pills, solutions, suspensions, ointments, lotions, creams, gels, pastes, sprays and aerosols. Liposomes and emulsions are well-known types of pharmaceutical formulations that can be used to deliver a pharmaceutical agent, particularly a hydrophobic pharmaceutical agent. Pharmaceutical compositions of the present invention generally include a pharmaceutically acceptable carrier such as an excipient, diluent and/or vehicle. Delayed release formulations of compositions and delayed release systems, such as semipermeable matrices of solid hydrophobic polymers can be used.

The term “pharmaceutically acceptable carrier” refers to a carrier which is suitable for use in a subject without undue toxicity or irritation to the subject and which is compatible with other ingredients included in a pharmaceutical composition.

Pharmaceutically acceptable carriers, methods for making pharmaceutical compositions and various dosage forms, as well as modes of administration are well-known in the art, for example as detailed in Pharmaceutical Dosage Forms: Tablets, eds. H. A. Lieberman et al., New York: Marcel Dekker, Inc., 1989; and in L. V. Allen, Jr. et al., Ansel's Pharmaceutical Dosage Forms and Drug Delivery Systems, 8th Ed., Philadelphia, Pa.: Lippincott, Williams & Wilkins, 2004; A. R. Gennaro, Remington: The Science and Practice of Pharmacy, Lippincott Williams & Wilkins, 21st ed., 2005, particularly chapter 89; and J. G. Hardman et al., Goodman & Gilman's The Pharmacological Basis of Therapeutics, McGraw-Hill Professional, 10th ed., 2001.

A solid dosage form for administration or for suspension in a liquid prior to administration illustratively includes capsules, tablets, powders, and granules. In such solid dosage forms, one or more active agents, is admixed with at least one carrier illustratively including a buffer such as, for example, sodium citrate or an alkali metal phosphate illustratively including sodium phosphates, potassium phosphates and calcium phosphates; a filler such as, for example, starch, lactose, sucrose, glucose, mannitol, and silicic acid; a binder such as, for example, carboxymethylcellulose, alginates, gelatin, polyvinylpyrrolidone, sucrose, and acacia; a humectant such as, for example, glycerol; a disintegrating agent such as, for example, agar-agar, calcium carbonate, plant starches such as potato or tapioca starch, alginic acid, certain complex silicates, and sodium carbonate; a solution retarder such as, for example, paraffin; an absorption accelerator such as, for example, a quaternary ammonium compound; a wetting agent such as, for example, cetyl alcohol, glycerol monostearate, and a glycol; an adsorbent such as, for example, kaolin and bentonite; a lubricant such as, for example, talc, calcium stearate, magnesium stearate, a solid polyethylene glycol or sodium lauryl sulfate; a preservative such as an antibacterial agent and an antifungal agent, including for example, sorbic acid, gentamycin and phenol; and a stabilizer such as, for example, sucrose, EDTA, EGTA, and an antioxidant.

Solid dosage forms optionally include a coating such as an enteric coating. The enteric coating is typically a polymeric material. Preferred enteric coating materials have the characteristics of being bioerodible, gradually hydrolyzable and/or gradually water-soluble polymers. The amount of coating material applied to a solid dosage generally dictates the time interval between ingestion and drug release. A coating is applied having a thickness such that the entire coating does not dissolve in the gastrointestinal fluids at pH below 3 associated with stomach acids, yet dissolves above pH 3 in the small intestine environment. It is expected that any anionic polymer exhibiting a pH-dependent solubility profile is readily used as an enteric coating in the practice of the present invention to achieve delivery of the active agent to the lower gastrointestinal tract. The selection of the specific enteric coating material depends on properties such as resistance to disintegration in the stomach; impermeability to gastric fluids and active agent diffusion while in the stomach; ability to dissipate at the target intestine site; physical and chemical stability during storage; non-toxicity; and ease of application.

Suitable enteric coating materials illustratively include cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, preferably formed from acrylic acid, methacrylic acid, methyl acrylate, ammonium methylacrylate, ethyl acrylate, methyl methacrylate and/or ethyl; vinyl polymers and copolymers such as polyvinyl pyrrolidone, polyvinyl acetate, polyvinylacetate phthalate, vinylacetate crotonic acid copolymer, and ethylene-vinyl acetate copolymers; shellac; and combinations thereof. A particular enteric coating material includes acrylic acid polymers and copolymers described for example U.S. Pat. No. 6,136,345.

The enteric coating optionally contains a plasticizer to prevent the formation of pores and cracks that allow the penetration of the gastric fluids into the solid dosage form. Suitable plasticizers illustratively include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, a coating composed of an anionic carboxylic acrylic polymer typically contains approximately 10% to 25% by weight of a plasticizer, particularly dibutyl phthalate, polyethylene glycol, triethyl citrate and triacetin. The coating can also contain other coating excipients such as detackifiers, antifoaming agents, lubricants (e.g., magnesium stearate), and stabilizers (e.g. hydroxypropylcellulose, acids or bases) to solubilize or disperse the coating material, and to improve coating performance and the coated product.

Liquid dosage forms for oral administration include one or more active agents and a pharmaceutically acceptable carrier formulated as an emulsion, solution, suspension, syrup, or elixir. A liquid dosage form of a composition of the present invention may include a colorant, a stabilizer, a wetting agent, an emulsifying agent, a suspending agent, a sweetener, a flavoring, or a perfuming agent.

For example, a composition for parenteral administration may be formulated as an injectable liquid. Examples of suitable aqueous and nonaqueous carriers include water, ethanol, polyols such as propylene glycol, polyethylene glycol, glycerol, and the like, suitable mixtures thereof, vegetable oils such as olive oil; and injectable organic esters such as ethyloleate. Proper fluidity can be maintained, for example, by the use of a coating such as lecithin, by the maintenance of a desirable particle size in the case of dispersions, and/or by the use of a surfactant, such as sodium lauryl sulfate. A stabilizer is optionally included such as, for example, sucrose, EDTA, EGTA, and an antioxidant.

For topical administration, a composition can be formulated for administration to the skin such as for local effect, and/or as a “patch” formulation for transdermal delivery. Pharmaceutical formulations suitable for topical administration include, for example, ointments, lotions, creams, gels, pastes, sprays and powders. Ointments, lotions, creams, gels and pastes can include, in addition to one or more active agents, a base such as an absorption base, water-removable base, water-soluble base or oleaginous base and excipients such as a thickening agent, a gelling agent, a colorant, a stabilizer, an emulsifying agent, a suspending agent, a sweetener, a flavoring, or a perfuming agent.

Transdermal formulations can include percutaneous absorption enhancers such as acetone, azone, dimethyl acetamide, dimethyl formamide, dimethyl sulfoxide, ethanol, oleic acid, polyethylene glycol, propylene glycol and sodium lauryl sulfate. Ionotophoresis and/or sonophoresis can be used to enhance transdermal delivery.

Powders and sprays for topical administration of one or more active agents can include excipients such as talc, lactose and one or more silicic acids. Sprays can include a pharmaceutical propellant such as a fluorinated hydrocarbon propellant, carbon dioxide, or a suitable gas. Alternatively, a spray can be delivered from a pump-style spray device which does not require a propellant. A spray device delivers a metered dose of a composition contained therein, for example, using a valve for regulation of a delivered amount.

Ophthalmic formulations of one or more active agents can include ingredients such as a preservative, a buffer and a thickening agent.

Suitable surface-active agents useful as a pharmaceutically acceptable carrier or excipient in the pharmaceutical compositions of the present invention include non-ionic, cationic and/or anionic surfactants having good emulsifying, dispersing and/or wetting properties. Suitable anionic surfactants include both water-soluble soaps and water-soluble synthetic surface-active agents. Suitable soaps are alkaline or alkaline-earth metal salts, non-substituted or substituted ammonium salts of higher fatty acids (C10-C22), e.g. the sodium or potassium salts of oleic or stearic acid, or of natural fatty acid mixtures obtainable form coconut oil or tallow oil. Synthetic surfactants include sodium or calcium salts of polyacrylic acids; fatty sulphonates and sulphates; sulphonated benzimidazole derivatives and alkylarylsulphonates. Fatty sulphonates or sulphates are usually in the form of alkaline or alkaline-earth metal salts, non-substituted ammonium salts or ammonium salts substituted with an alkyl or acyl radical having from 8 to 22 carbon atoms, e.g. the sodium or calcium salt of lignosulphonic acid or dodecylsulphonic acid or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Suitable sulphonated benzimidazole derivatives preferably contain 8 to 22 carbon atoms. Examples of alkylarylsulphonates are the sodium, calcium or alcanolamine salts of dodecylbenzene sulphonic acid or dibutyl-naphthalene sulphonic acid or a naphthalene-sulphonic acid/formaldehyde condensation product. Also suitable are the corresponding phosphates, e.g. salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g. phosphatidylethanolamine, phosphatidylserine, phosphatidylglycerine, lysolecithin, cardiolipin, dioctanylphosphatidylcholine, dipalmitoylphosphatidyl-choline and their mixtures.

Suitable non-ionic surfactants useful as pharmaceutically acceptable carriers or excipients in the pharmaceutical compositions of the present invention include polyethoxylated and polypropoxylated derivatives of alkylphenols, fatty alcohols, fatty acids, aliphatic amines or amides containing at least 12 carbon atoms in the molecule, alkylarenesulphonates and dialkylsulphosuccinates, such as polyglycol ether derivatives of aliphatic and cycloaliphatic alcohols, saturated and unsaturated fatty acids and alkylphenols, said derivatives preferably containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with poylypropylene glycol, ethylenediaminopolypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol-polyethoxyethanol, castor oil polyglycolic ethers, polypropylenei polyethylene oxide adducts, tributylphenoxypolyethoxyethanol, polyethyleneglycol and octylphenoxypolyethoxyethanol. Fatty acid esters of polyethylene sorbitan (such as polyoxyethylene sorbitan trioleate), glycerol, sorbitan, sucrose and pentaerythritol are also suitable non-ionic surfactants.

Suitable cationic surfactants useful as pharmaceutically acceptable carriers or excipients in the pharmaceutical compositions of the present invention include quaternary ammonium salts, preferably halides, having 4 hydrocarbon radicals optionally substituted with halo, phenyl, substituted phenyl or hydroxy; for instance quaternary ammonium salts containing as N-substituent at least one C8-C22 alkyl radical (e.g. cetyl, lauryl, palmityl, myristyl, oleyl and the like) and, as further substituents, unsubstituted or halogenated lower alkyl, benzyl and/or hydroxy-lower alkyl radicals.

A more detailed description of surface-active agents suitable for this purpose may be found for instance in “McCutcheon's Detergents and Emulsifiers Annual” (MC Publishing Crop., Ridgewood, N.J., 1981), “Tensid-Taschenbuch”, 2nd ed. (Hanser Verlag, Vienna, 1981) and “Encyclopaedia of Surfactants (Chemical Publishing Co., New York, 1981).

Structure-forming, thickening or gel-forming agents may be included into the pharmaceutical compositions and combined preparations of the invention. Suitable such agents are in particular highly dispersed silicic acid, such as the product commercially available under the trade name Aerosil; bentonites; tetraalkyl ammonium salts of montmorillonites (e.g., products commercially available under the trade name Bentone), wherein each of the alkyl groups may contain from 1 to 20 carbon atoms; cetostearyl alcohol and modified castor oil products (e.g. the product commercially available under the trade name Antisettle).

In particular aspects, a pharmaceutically acceptable carrier is a particulate carrier such as lipid particles including liposomes, micelles, unilamellar or mulitlamellar vesicles; polymer particles such as hydrogel particles, polyglycolic acid particles or polylactic acid particles; inorganic particles such as calcium phosphate particles such as described in for example U.S. Pat. No. 5,648,097; and inorganic/organic particulate carriers such as described for example in U.S. Pat. No. 6,630,486.

A particulate pharmaceutically acceptable carrier can be selected from among a lipid particle; a polymer particle; an inorganic particle; and an inorganic/organic particle. A mixture of particle types can also be included as a particulate pharmaceutically acceptable carrier.

A particulate carrier is typically formulated such that particles have an average particle size in the range of about 1 nm-10 microns. In particular aspects, a particulate carrier is formulated such that particles have an average particle size in the range of about 1 nm-100 nm.

Detailed information concerning customary ingredients, equipment and processes for preparing dosage forms is found in Pharmaceutical Dosage Forms: Tablets, eds. H. A. Lieberman et al., New York: Marcel Dekker, Inc., 1989; and in L. V. Allen, Jr. et al., Ansel's Pharmaceutical Dosage Forms and Drug Delivery Systems, 8th Ed., Philadelphia, Pa.: Lippincott, Williams & Wilkins, 2004; A. R. Gennaro, Remington: The Science and Practice of Pharmacy, Lippincott Williams & Wilkins, 21st ed., 2005, particularly chapter 89; and J. G. Hardman et al., Goodman & Gilman's The Pharmacological Basis of Therapeutics, McGraw-Hill Professional, 10th ed., 2001.

Commercial packages according to aspects of the present invention include an AKT inhibitor and a WEE1 inhibitor, formulated in combination or separately. Instructions for administering the AKT inhibitor and the WEE1 inhibitor are included according to aspects of the invention.

Commercial packages according to aspects of the present invention include 1) AZD5363 and/or GDC0068 and 2) MK1775, formulated in combination or separately.

One or more ancillary components is optionally included in commercial packages of the present invention, such as a buffer or diluent.

Methods of treating cancer are provided according to aspects of the present invention which include obtaining a first sample containing or suspected of containing cancer cells from the subject prior to administering the combination of the AKT inhibitor and the WEE1 inhibitor, obtaining a second sample containing or suspected of containing cancer cells from the subject after administering the combination of the AKT inhibitor and the WEE1 inhibitor; and assaying the first and second samples for one or more markers of apoptosis and/or for activity of a mitogen-activated protein kinase-signaling pathway, thereby monitoring effectiveness of administering the combination of the AKT inhibitor and the WEE1 inhibitor.

Methods of treating cancer are provided according to aspects of the present invention which include obtaining a first sample containing or suspected of containing cancer cells from the subject prior to administering the combination of the AKT inhibitor and the WEE1 inhibitor, obtaining a second sample containing or suspected of containing cancer cells from the subject after administering the combination of the AKT inhibitor and the WEE1 inhibitor; and assaying the first and second samples for one or more markers of AKT dysregulation, thereby monitoring effectiveness of administering the combination of the AKT inhibitor and the WEE1 inhibitor.

Methods of treating cancer are provided according to aspects of the present invention which include obtaining a first sample containing or suspected of containing cancer cells from the subject prior to administering the combination of the AKT inhibitor and the WEE1 inhibitor, obtaining a second sample containing or suspected of containing cancer cells from the subject after administering the combination of the AKT inhibitor and the WEE1 inhibitor; and assaying the first and second samples for p53 expression and/or expression of a downstream factor in the p53 transcriptional pathway selected from the group consisting of: chloride channel accessory 2 (CLCA2), nectin cell adhesion molecule 4 (PVRL4), sulfatase 2 (SULF2), cyclin dependent kinase inhibitor 1A (CDKN a). BTG anti-proliferation factor 2 (BTG2), actin, alpha 2, smooth muscle, aorta (ACTA2), tumor protein p53 (TP53), ferredoxin reductase (FDXR), growth differentiation factor 15 (GDF15), insulin like growth factor binding protein 5 (IGFBP5) and ADAM metallopeptidase domain 19 (ADAM19), wherein an increase in p53 expression, and/or a downstream factor selected from the group consisting of: CLCA2, PVRL4, SULF2, CDKN Ia, BTG2, ACTA2, TP53, FDXR, GDF15, IGFBP5 and ADAM19, is an indicator of an anti-cancer cell effect of treatment with the combination of the AKT inhibitor and the WEE1 inhibitor, thereby monitoring effectiveness of administering the combination of the AKT inhibitor and the WEE1 inhibitor.

Methods of treating cancer are provided according to aspects of the present invention which include obtaining a first sample containing or suspected of containing cancer cells from the subject prior to administering the combination of the AKT inhibitor and the WEE1 inhibitor, obtaining a second sample containing or suspected of containing cancer cells from the subject after administering the combination of the AKT inhibitor and the WEE1 inhibitor; and assaying the first and second samples for forkhead box MI (FOXM1) expression and/or expression of an associated downstream protein in this transcriptional pathway selected from the group consisting of: thymopoietin (TMPO), acidic nuclear phosphoprotein 32 family member E (ANP32E), structural maintenance of chromosomes 4 (SMC4), kinesin family member 20B (KIF20B), abnormal spindle microtubule assembly (ASPM), DEP domain containing 1 (DEPDC1), non-SMC condensin I complex subunit G (NCAPG) and centromere protein E (CENPE), wherein a decrease in FOXM1 expression and/or an associated downstream protein selected from the group consisting of: TMPO, ANP32E, SMC4, KIF20B, ASPM, DEPDC1, NCAPG and CENPE, is an indicator of an anti-cancer cell effect of treatment with the combination of the AKT inhibitor and the WEE1 inhibitor, thereby monitoring effectiveness of administering the combination of the AKT inhibitor and the WEE inhibitor.

Assays for expression of one or more markers of apoptosis, activity of a mitogen-activated protein kinase-signaling pathway, AKT dysregulation, p53 expression and/or expression of a downstream factor in the p53 transcriptional pathway selected from the group consisting of: CLCA2, PVRL4, SULF2, CDKN1a, BTG2, ACTA2, TP53, FDXR, GDF15, IGFBP5 and ADAM19; and/or FOXM1 expression and/or expression of an associated downstream protein in the FOXM1 transcriptional pathway selected from the group consisting of: TMPO, ANP32E, SMC4, KIF20B, ASPM, DEPDC1, NCAPG and CENPE, are performed using standard techniques such as nucleic acid assays, spectrometric assays, immunoassays and functional assays. One or more standards and/or controls can be used to allow quantitative determination of target marker in a sample.

Standards and controls suitable for assays are well-known in the art and the standard and/or control used can be any appropriate standard and/or control.

Methods of treatment of a subject having, or at risk of having cancer characterized by constitutive activation of MAP (mitogen-activated protein) kinase-signaling pathway through BRAF, KIT and/or RAS mutations are provided according to aspects of the present invention which include administering a combination of an AKT inhibitor and a WEE1 inhibitor as a combination formulation or separately, wherein administration of the combination provides a synergistic effect.

^{V600E}BRAF is the most frequent genetic alteration occurring in 50% of sporadic melanoma, see Sullivan R J et al., Journal of Skin Cancer 2011; 2011:423239. Mutations in BRAF are also common in many other cancers and ^{V600E}BRAF is commonly found in colorectal and thyroid cancers. Mutations in KIT and N-RAS also activate MAPK signaling in 2 to 6% and 15 to 20% of cutaneous melanomas respectively, which like BRAF mutation regulates diverse cellular processes including proliferation, survival and metastases, see Sullivan R J et al., Journal of Skin Cancer 2011, 2011:423239; and Flaherty K T et al., The New England Journal of Medicine, 2010, 363(9):809-19.

Targeting WEE1 has been shown to be effective especially on mutant cancer cell lines. Methods of treatment of a subject having, or at risk of having cancer characterized by 1) constitutive activation of MAP (mitogen-activated protein) kinase-signaling pathway through BRAF, KIT and/or RAS mutations and/or 2) one or more p53 mutations, are provided according to aspects of the present invention which include administering a combination of an AKT inhibitor and a WEE1 inhibitor as a combination formulation or separately, wherein administration of the combination provides a synergistic effect.

The mutation status of BRAF, KIT, RAS and/or p53 can be assayed in a test sample obtained from a subject.

The mutation status of BRAF, KIT, RAS and/or p53 can be assayed by any of various methodologies including, but not limited to, protein or peptide sequencing, nucleic acid assay and immunoassay.

Assays for detecting BRAF, KIT, RAS and/or p53 nucleic acids, particularly mRNA or cDNA, include, but are not limited to, sequencing; polymerase chain reactions (PCR) such as RT-PCR; dot blot; in situ hybridization; Northern blot; and RNase protection.

A test sample can be any biological fluid, cell or tissue of a subject that includes or is suspected of including cancer cells or circulating DNA derived from cancer cells, illustratively including blood, plasma, serum, urine, saliva, ascites, cerebrospinal fluid, cerebroventricular fluid, pleural fluids, pulmonary and bronchial lavage samples, mucous, sweat, tears, semen, bladder wash samples, amniotic fluid, lymph, peritoneal fluid, synovial fluid, bone marrow aspirate, tumor cells or tissue, organ cells or tissue, such as biopsy material.

Immunoassay methods can be used to assay BRAF, KIT, RAS and/or p53 mutation status in a sample, including, but not limited to, enzyme-linked immunosorbent assay (ELISA), enzyme-linked immunofiltration assay (ELIFA), flow cytometry, immunoblot, immunoprecipitation, immunohistochemistry, immunocytochemistry, luminescent immunoassay (LIA), fluorescent immunoassay (FIA), and radioimmunoassay.

Embodiments of inventive compositions and methods are illustrated in the following examples. These examples are provided for illustrative purposes and are not considered limitations on the scope of inventive compositions and methods.

### EXAMPLES

Statistical Analyses

Statistical analyses were performed using Prism 4.0 GraphPad Software. Data were subjected to Chou-Talalay method for determining the combination index using CalcuSyn software and combination index (CI) values plotted against fraction affected as described in detail in Chou et al, Advances in Enzyme Regulation 1984; 22:27-55. Using this approach, combination index values of <0.9 are synergistic, >1.1 are antagonistic, and values 0.9-1.1 are nearly additive. For comparison between two groups, Student t test (2 tailed) was used. One-way Analysis Of Variance (ANOVA) was used for group wise comparisons, followed by the Tukey's post hoc test. Results represent at least two to three independent experiments and are shown as averages t SEM. Results with a P value less than 0.05 (95% CI) were considered significant. Number of asterisks in the figures indicates the level of statistical significance as follows: *P<0.05, **P<0.01, ***P<0.001.

Cell Lines and Culture Conditions

Metastatic Melanoma Cell Lines, UACC 903 and the 1205 Lu Cell Line, Both contain ^{V600E}B-Raf. Wild-type B-Raf protein containing cell line, C8161.C19 was also used. Breast cancer cell lines MCF7 and T47D, prostate cancer cell lines PC3 amd LNCAP, sarcoma cell line HT1080, glioblastoma cell line U87MG, T-cell acute lymphoblastic leukaemia (T-ALL) cell line Nalm6, colon cancer cell line Caco2, lung cancer cell line A549 and liver cancer cell line HepG2 were used in some experiments. All cell lines were maintained in DMEM (Life Technologies, Grand Island, N.Y.) supplemented with 1% GlutaMAX from Gibco (Life Technologies, Grand Island, N.Y.) and 10% FBS (HyClone, Logan, Utah) in a 37° C. humidified 5% CO₂ atmosphere incubator and periodically monitored for genotypic characteristics, phenotypic behavior and tumorigenic potential.

Western Blotting

Cell lysates were collected 48-96 hours after siRNA transfection or drug treatment by washing plates with PBS followed by addition of RIPA lysis buffer containing Halt Protease & Phosphatase Inhibitor Cocktail (Thermo Scientific, Rockford, Ill., USA). Lysates were centrifuged (10,000×g) for 10 minutes at 4° C. to remove cell debris. Protein concentration was measured using the bicinchoninic acid assay kit (Thermo Scientific, Rockford, Ill., USA). 25 μg of respective lysates were loaded onto 4-12% Bis-Tris NuPAGE gels (Life Technologies, Grand Island, N.Y.) and run in an XCell SureLock Mini-Cell gel apparatus (Life Technologies, Grand Island, N.Y.), transferred to polyvinylidene difluoride membranes (PVDF) (Pall Corporation, Port Washington, N.Y.), probed with respective primary antibody followed by horseradish peroxidase-conjugated secondary antibody, and exposed using ECL Western Blotting Substrate (Thermo Scientific, Rockford, Ill., USA) or Supersignal West Femto Chemiluminescent Substrate (Thermo Scientific, Rockford, Ill., USA). Primary antibodies used: p21 (sc-756), p27 (sc-528), PLK1 (sc-17783), p53 (sc-6243) and ERK2 (sc-1647) from Santa Cruz Biotechnology (Dallas, Tex.), pAKT (S473 in AKT1, S474 in AKT2 and S472 in AKT3, #9271), AKT3 (#3788), Total AKT (#4685), pWEE1 (#4910), WEE1 (#4936), p-CDC2 (Y15) (#9111), p-CDC2 (T161) (#9114), CDC2 (#9112) CHK1 (#2345), pCHK1 (#2344), pRB (S807/811) (#9308), pRB (S795) (#9301), pRB (S780) (#9307), RB (#9309), FOXM1 (#5436), p-Histone H2AX (S139) (#2577) and Cleaved PARP (#9541) from Cell Signaling (Danvers, Mass.). Secondary antibodies goat anti-rabbit IgG-HRP (sc-2004) and goat anti-mouse IgG-HRP (sc-2005) were purchased from Santa Cruz Biotechnology, Dallas, Tex.

Small Interfering RNA (siRNA) Transfection

siRNA was introduced into melanoma cells via nucleofection using an Amaxa nucleofector with solution R/program K-17 for UACC 903 and 1205 Lu cells, as described in detail in Sharma et al, A J P, 2013; 182(4): 1151-62; Stahl et al., Cancer Res., 2004, 64:7002-7010; and in Sharma et al, Cancer Research 2006; 66(16): 8200-9. Nucleofection efficiency was >90% with 80-90% cell viability. Following siRNA transfection, cells were plated and allowed to recover for 2 days and were then re-plated in 96-well plates to assess viability or cells were harvested for protein knockdown studies by Western blotting.

Duplexed Stealth siRNA (Invitrogen) sequences for scrambled (control), ^{V600E}BRAF, WEE1, AURKB, GSK3A and TPK1 were:

AURKB#1,
(SEQ ID NO: 1)
5′-CCAACAUCCUGCGUCUCUACAACUA-3′
AURKB#2,
(SEQ ID NO: 2)
5′-GGAUCUGUGGUGCAUUGGAGUGCUU-3
AURKB#3,
(SEQ ID NO: 3)
5′-ACUUGUCCUCAUGAG-CCGCUCCAAU-3′
WEE1#1,
(SEQ ID NO: 4)
5′-GGGCAUCCAACAAAGUUAUGUUUAA-3′
WEE1#2,
(SEQ ID NO: 5)
5′-CCAGAGUAAUAGAACAUCUCGACUU-3′
WEE1#3,
(SEQ ID NO: 6)
5′-CCUCUUCCGAGAAAUGGAGAUCAAU-3′
GSK3A#1,
(SEQ ID NO: 7)
5′-CCAAGGCCAAGUUGACCAUCCCUAU-3′
GSK3A#2,
(SEQ ID NO: 8)
5′-GGAGUUCAAGUUCCCUCAGAUUAAA-3′
GSK3A#3,
(SEQ ID NO: 9)
5′-CCAGGGAACUAGUCGCCAUCAAGAA-3′
TPK1#1,
(SEQ ID NO: 10)
5′-GCUACUAAGGGAUGUGAGCUCAUUU-3′
TPK1#2,
(SEQ ID NO: 11)
5′-CCACACUGACUUUACUAGUGCCUU-3′
TPK1#3,
(SEQ ID NO: 12)
5′-UGGAACAUUGGUCAGUACUUCCAAU-3′
AKT3-
(SEQ ID NO: 13)
GGA CUA UCU ACA UUC CGG AAA GAU U
^{V600E}B-RAF-
(SEQ ID NO: 14)
AUC GAG AUU UCU CUG UAG CUA GAC C
SCRAMBLED-
(SEQ ID NO: 15)
AAU UCU CCG AAC GUG UCA CGU GAG A

siRNA screening and synergy analysis of cultured cells to identify kinases synergizing with AKT3

siRNA screening was performed as described in Sharma et al, the American Journal of Pathology 2013; 182(4): 1151-62 and in Madhunapantula et al, Pigment Cell & Melanoma Research 2013; 26(6): 218-21. For synergy studies, 6.25-100 pmole of siRNA targeting AKT3 and five kinases (WEE1, aurora kinase B (AURKB), glycogen synthase kinase-3 alpha (GSK3A), thiamin pyrophosphokinase 1 (TPK1) or mutant B-raf proto oncogene (^{V600E} BRAF) that were identified from the screen were introduced into 1×10⁶ melanoma cells alone or in combination using an Amaxa nucleofector. Two days post transfection, cells were trypsinised and 1×10⁴ cells/well in 100 μL of serum-free media were plated in to 96-well plates with six to eight replicates for each siRNA and for each Scrambled siRNA control. Cells were grown for 72 hours and viability of cells was measured using MTS assay and percentage decrease in viability compared with the scrambled siRNA control. The data were subjected to Chou-Talalay analysis for determining the combination index using CalcuSyn software (Biosoft, Cambride, UK) and CI values were plotted against fraction affected. Using this approach, combination index values of <0.9 are synergistic, >1.1 are antagonistic and values in the range of 0.9-1.1 are additive.

Targeting AKT3 and WEE1 Synergistically Inhibits Melanoma Cell Survival

To identify druggable targets that enhance the efficacy of AKT3 inhibition, a screen was undertaken to identify kinases that could be targeted to inhibit melanoma tumor development. An siRNA library was screened to identify important kinases involved in melanoma cell viability. The screen was undertaken using a pool of three siRNAs targeting each of the 636 kinases (totaling 1908 individual siRNAs). siRNAs were nucleofected into the UACC 903 melanoma cell line using the AMAXA 96-well shuttle transfection system and 5 days later, viability of cells was measured by MTS assay. Results were compared to the average of high-, medium-, and low-GC content scrambled siRNA transfected cells. From this primary screen, 34 kinases were identified as potential hits that are able to reduce viability of UACC 903 cells more significantly than the set experimental cut-off i.e. 15% growth inhibition, see Table I.

TABLE I
Kinases identified from a siRNA screen that regulate melanoma cell survival

| Genes Identified In Primary Screen | | | | | | |
|---|---|---|---|---|---|---|
| (3 pooled siRNA, transfected using | | | | | | |
| AMAχA 96 well shuttle system) | | | | | | |
| Gene | | % of Growth | Secondary Screen | Tertiary Screen | Validation in | In vivo |
| Symbol | Gene Name | Inhibition | (3 pooled siRNAs) | (3 individual siRNAs) | 2nd cell line | efficacy |
| PDPK1 | 3-phosphoinositide | 19 | ✓ | x | | |
| | dependent protein | | | | | |
| | kinase-1 | | | | | |
| AAK1 | AP2 associated | 20 | ✓ | x | | |
| | kinase 1 | | | | | |
| ARKB | Aurora kinase B | 21 | ✓ | ✓ | ✓ | ✓ |
| CKMT2 | Creatine kinase | 16.9 | ✓ | x | | |
| | mitrochondrial-2 | | | | | |
| GSK3A | Glycogen synthase | 31.3 | ✓ | ✓ | ✓ | ✓ |
| | kinase 3 alpha | | | | | |
| MARK1 | Microtubule affinity- | 32 | ✓ | x | | |
| | regulating kinase | | | | | |
| MAPK1 | Mitogen-activated | 23 | ✓ | x | | |
| | protein kinase 1 | | | | | |
| MAPK13 | Mitogen-activated | 33 | ✓ | x | | |
| | protein kinase 13 | | | | | |
| MAP2K1 | Mitogen-activated | 35.2 | ✓ | x | | |
| | protein kinase | | | | | |
| | kinase | | | | | |
| NAGS | N-acetylglutamate | 18.8 | ✓ | x | | |
| | synthase | | | | | |
| TPK1 | Thiamin pyrophospho | 21.3 | ✓ | ✓ | ✓ | ✓ |
| | kinase 1 | | | | | |
| TYK2 | Tyrosine kinase 2 | 33 | ✓ | x | | |
| BRAF | v-raf murine sarcoma | 57.4 | ✓ | ✓ | ✓ | ✓ |
| | viral oncogene | | | | | |
| | homolog B1 | | | | | |
| WEE1 | WEE1 homolog | 23.1 | ✓ | ✓ | ✓ | ✓ |
| | (S. pombe) | | | | | |

For the secondary screen, 34 kinases identified from the primary screen were again introduced into melanoma cell UACC 903 via nucleofection and 14 of the initial hits were validated, listed in Table I.

A tertiary screen was then conducted to determine if individual siRNA to each target had a similar inhibitory effect to the pooled siRNA used in the previous screens. A tertiary screen required that at least two of the three siRNAs targeting different regions of respective mRNAs decrease UACC 903 cell viability. Based on this criterion, AURKB, WEE1, GSK3A, TPK1 and BRAF were identified as potential targets that were able to reduce the proliferative potential of UACC 903 melanoma cells. The potential of these targets was then confirmed in two additional melanoma cell lines (A375M and 1205 Lu) to show similar growth inhibitory effects. Subsequently, siRNA mediated knockdown of these kinases was used to measure the inhibitory effect on xenografted melanoma tumor development. Results of the screen and subsequent validation of WEE1, GSK3A, TPK1 and AURKB are shown in Table I.

AKT3 was co-targeted with the identified kinases from the screen. Viability of UACC 903 cells following siRNA mediated knockdown of AKT3 alone or in combination with WEE1, GSK3A, AURKB, TPK1 and BRAF was examined by MTS assay. 100 pmole of siRNA to AKT3 was combined with increasing amounts of each of the validated kinases and viable cells quantitated. The Chou-Talalay method for determining the combination index (CI) was used to determine synergy when targeting AKT3 and AURKB. AKT3 and WEE1, AKT3 and GSK3A, or AKT3 and TPK1. The combination index (CI) values (a measure of the strength of association between two targets) were calculated using CalcuSyn software. Using this approach, combination index values <0.9 are synergistic, >1.1 is antagonistic, and values 0.9 to 1.1 are additive. The data indicated strong synergism between AKT3 and WEE1 (CI value 0.095) inhibition, see Table II.

TABLE II
Analysis of synergy between knockdown of AKT3 and
validated kinase hits from siRNA screen using Calcusyn software.

| Kinase target identified | siAKT3 | CI | |
|---|---|---|---|
| in the screen (pmole) | (pmole) | value | Result |
| ^{V600E}BRAF (3) | 200 | 0.909 | Additive |
| WEE1 (12.5) | 100 | 0.095 | Highly synergistic |
| GSK3A (50) | 100 | 0.267 | Synergistic |
| AURKB (100) | 100 | 0.986 | Additive |
| TPK1 (100) | 100 | 1.12 | Antagonistic |

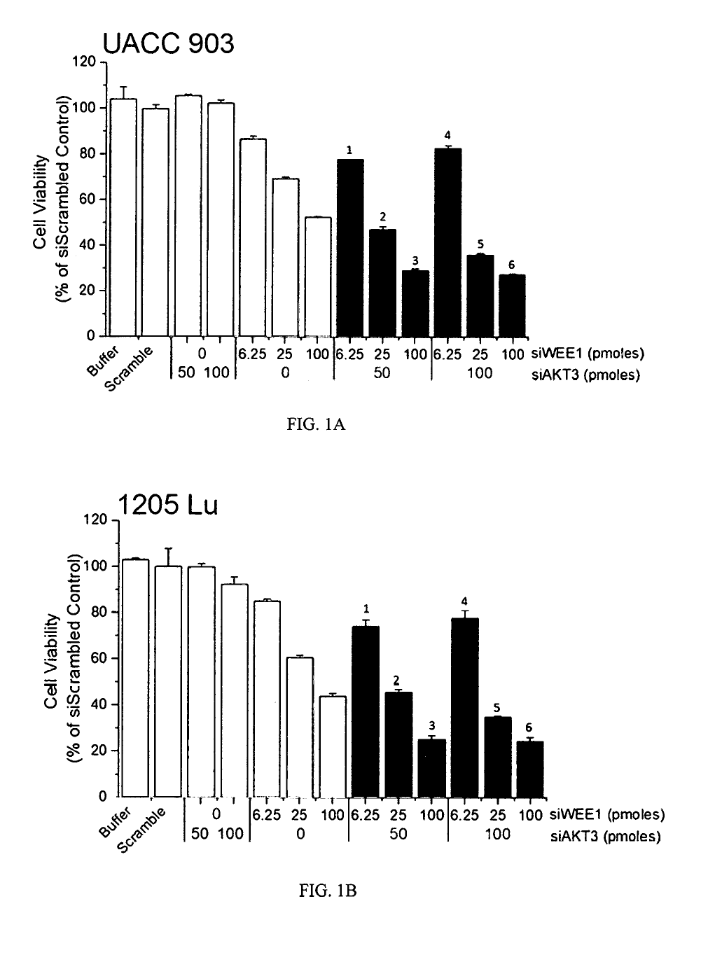

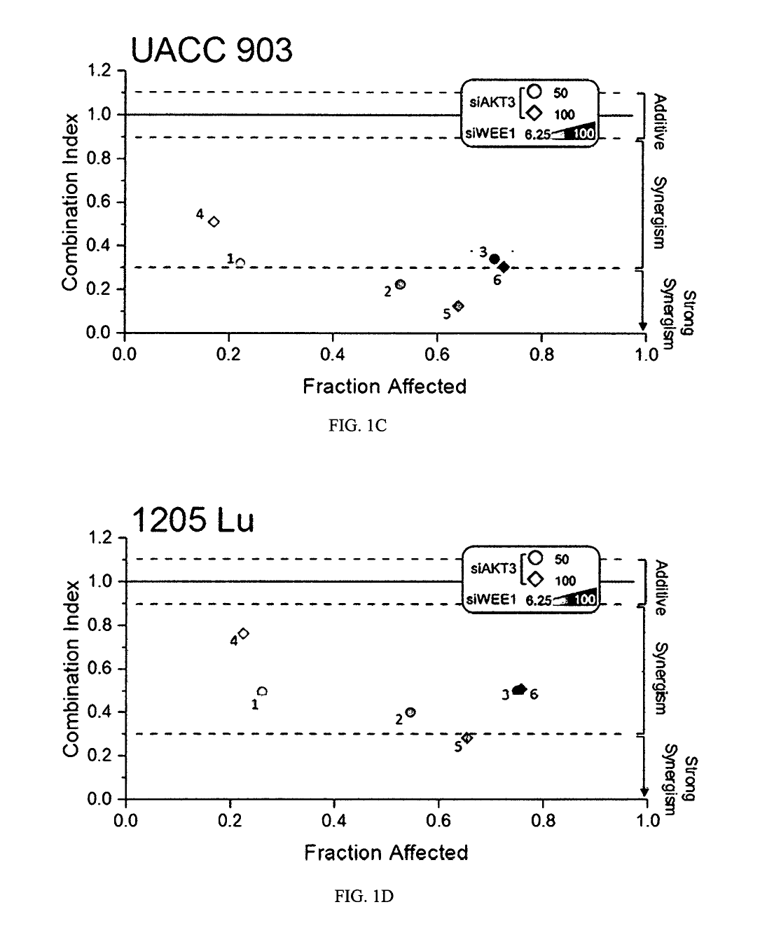

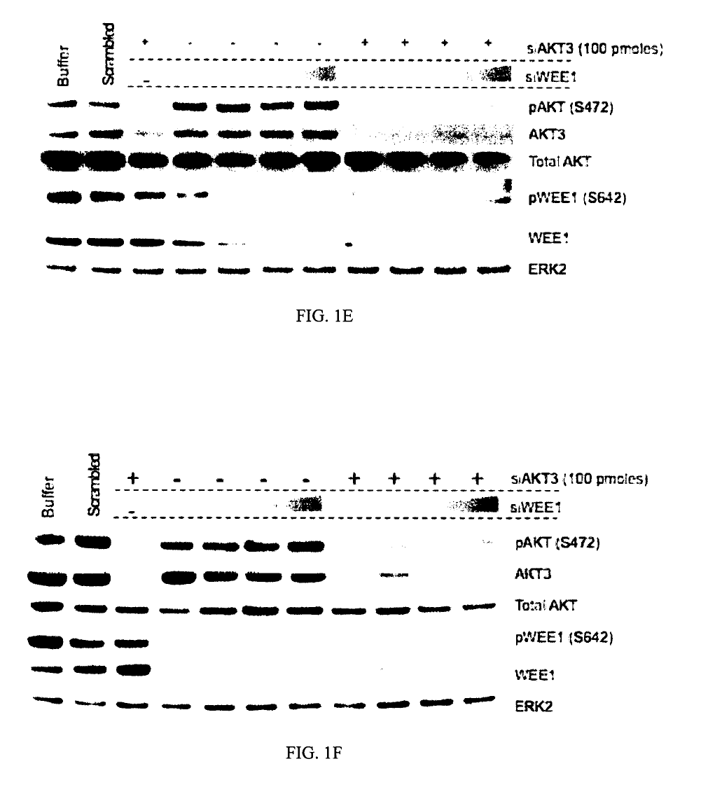

Nucleofection of 50 or 100 pmole of siRNA targeting AKT3 with increasing concentrations of WEE1 siRNA (6.25, 12.5, 25, 50 and 100 pmole) into UACC 903 and 1205 Lu cells had a measured effect on cell viability. Cellular viability was measured after 3 days of growth in serum free medium and dose response curves of siRNA are shown in FIGS. 1A and 1B for UACC 903 and 1205 Lu melanoma cell lines, respectively. Columns; mean (n=6-8); error bars show SEM.

Analyses of the combination index (CI) for the knockdown of AKT3 and WEE1 showed a synergistic relationship in both UACC 903 and 1205 Lu melanoma cell lines, with all concentrations of WEE1 siRNA, FIGS. 1C and 1D, respectively. Numbers associated with bars in FIGS. 1A and 1B correspond to the same number associated with a circle or diamond marker in FIGS. 1C and 1D, respectively.

An example of protein knockdown efficiencies following nucleofection was demonstrated by Western blotting. SiRNA-mediated knockdown of AKT3 and WEE1 protein levels. SiRNA to AKT3 alone or in combination with increasing amounts of WEE1 siRNA was introduced into melanoma cells via nucleofection and protein levels measured 2 days later. ERK2 served as a control for equal protein loading. Western blot analysis was reproduced at least twice, FIGS. 1E and 1F. siRNA for both AKT3 and WEE1 significantly decreased total and phosphorylated (active) levels of respective targets.

Synergy Studies with Pharmacological Agents

WEE1 inhibitor MK1775 (ChemieTek, Indianapolis, Ind.) and AKT inhibitors GDC0068 (ChemieTek, Indianapolis, Ind.) or AZD5363 (ChemieTek, Indianapolis, Ind.) were dissolved in DMSO. Cytotoxicity of these agents alone or in combination in melanoma cells was measured by plating 2.5 to 5×10³ cells into 96-well plates followed by growth for 24 to 48 hours in a humidified 37° C. cell culture incubator. Next, stock solutions of MK1775 and GDC0068 or AZD5363 were added to the media and cells were treated for 48 hours at concentrations ranging from 0.125 to 20 micromolar. Control cells were treated with vehicle (DMSO) alone. Cytotoxicity was measured using the CellTiter 96 Aqueous Non-Radioactive Cell Proliferation Assay (Promega). The combined effects of MK1775 and GDC0068 were quantified using CalcuSyn software.

Colony Formation Assay

UACC 903 cells were plated at a density of 500 cells per well in a 6-well plate in duplicate. 4 days later wells were treated with MK1775 and GDC0068 alone or in combination at concentrations ranging from 0.125 to 2.5 micromolar for 24 hours and subsequently grown in normal culture medium. Cells were to grown for 10 days, then the medium was aspirated, the wells were washed with phosphate buffered saline (PBS) and the colonies were stained with 0.05% crystal violet/l % methanol solution/l % formaldehyde solution and plates photographed.

Pharmacological Cotargeting of AKT3 and WEE1 Synergistically Inhibits Melanoma Cell Survival

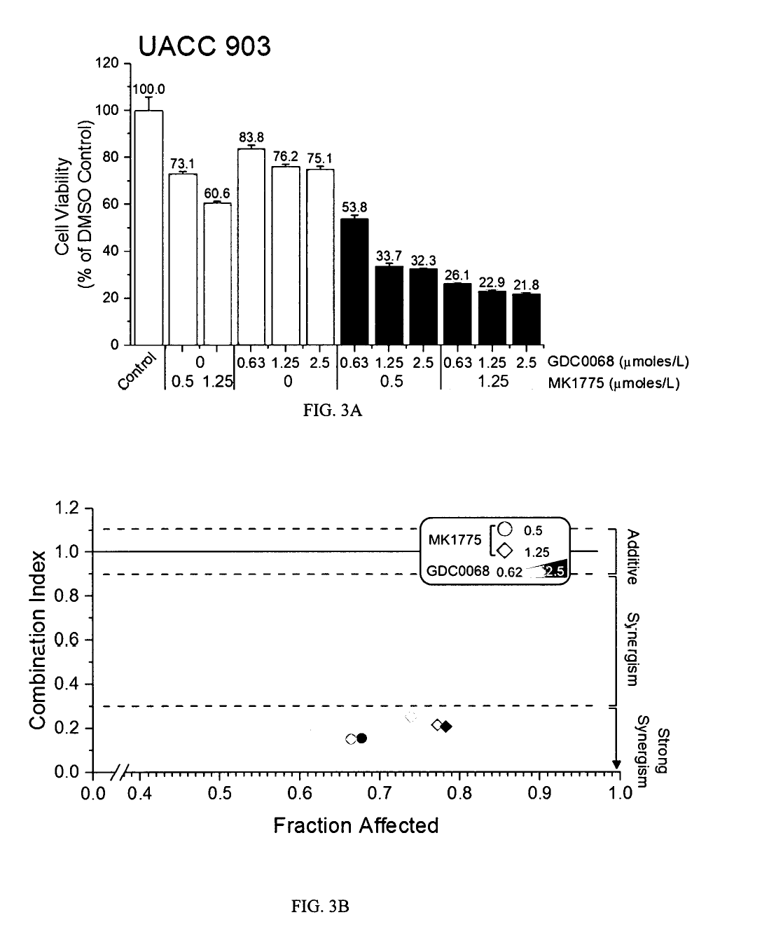

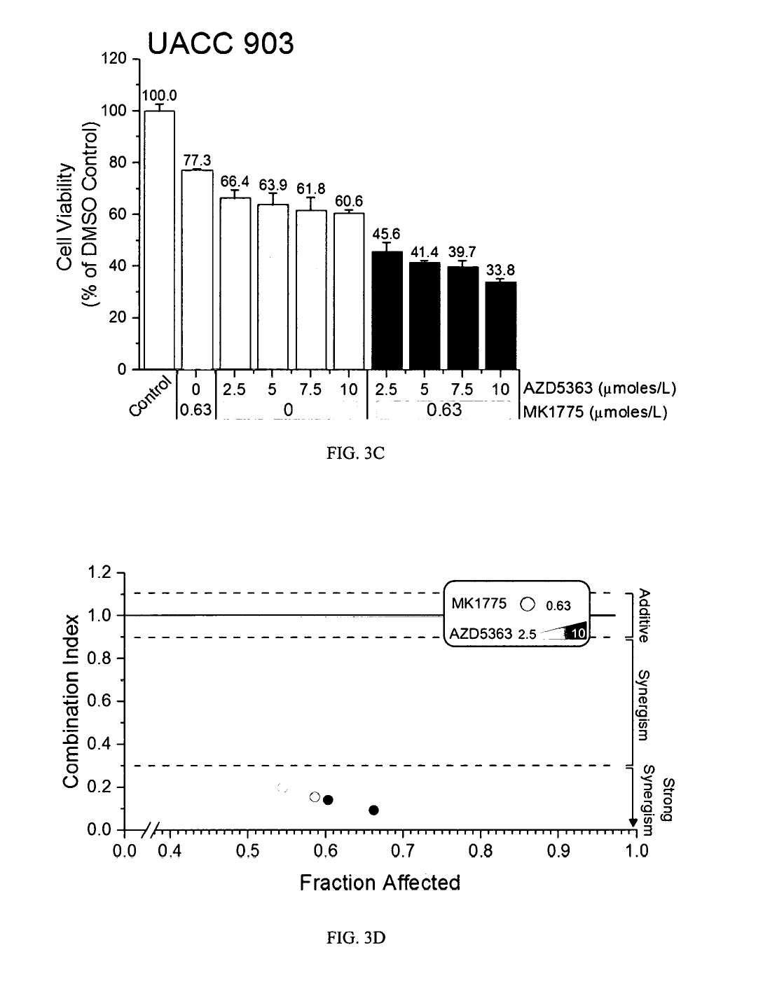

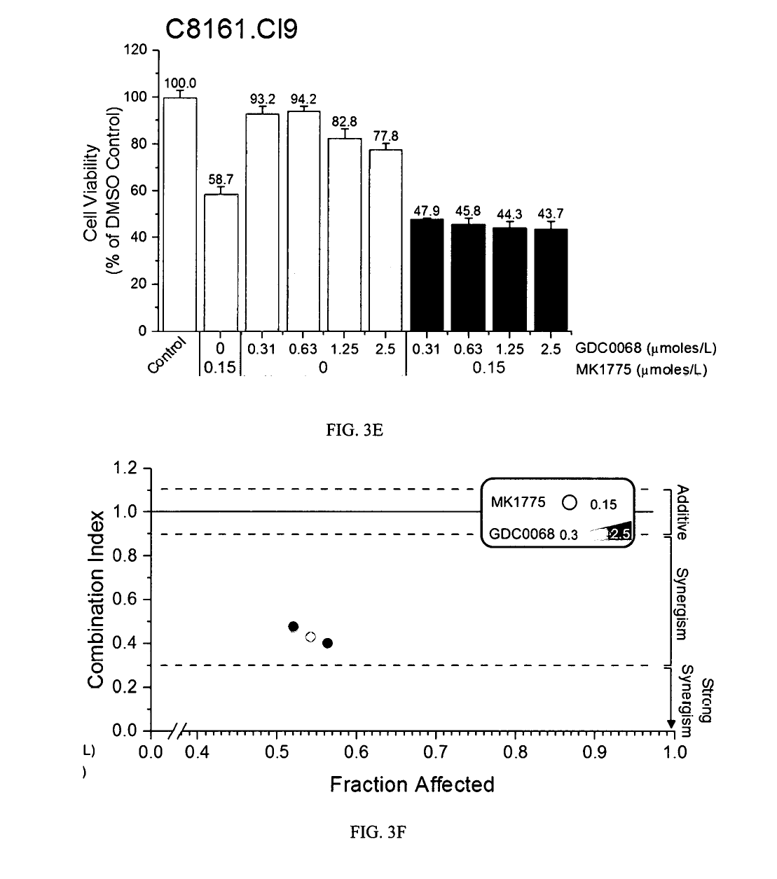

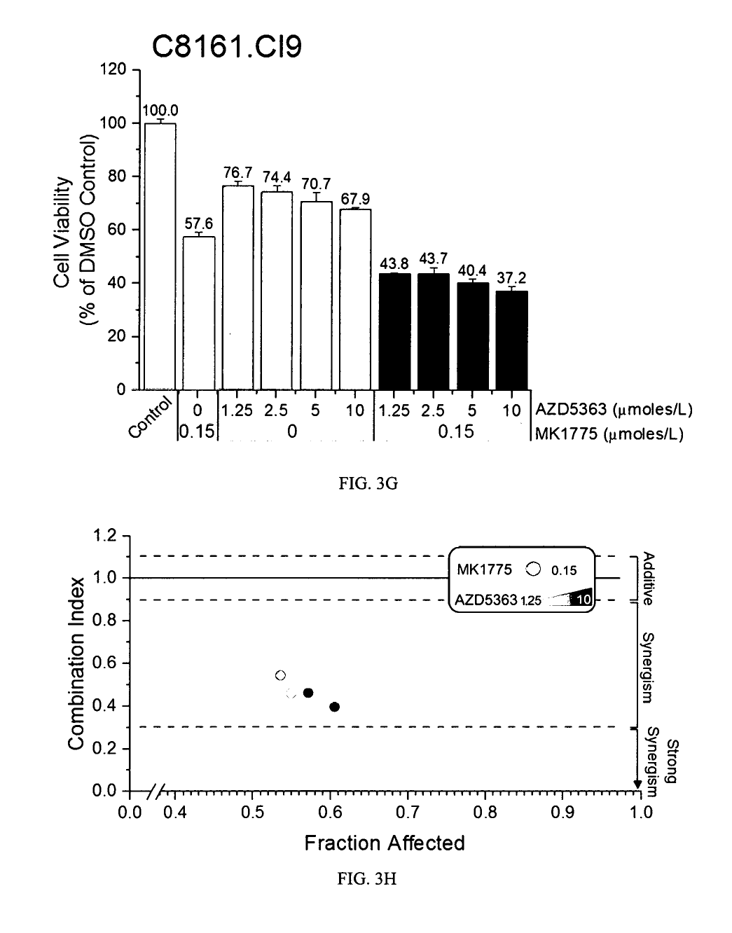

Since siRNA-mediated targeting of AKT3 and WEE) synergistically inhibited melanoma cell growth, combination of small molecule inhibitors targeting AKT (AZD5363 or GDC0068) and WEE1 (MK1775) were investigated. In both mutant ^{V600E}BRAF and wild-type BRAF melanoma cells, UACC 903 and C8161.C19 cells, respectively, a combination of a WEE1 inhibitor and an AKT inhibitor synergistically inhibited cell survival (FIGS. 3A-3H, Columns, mean (n=6-8); error bars show SEM.

In UACC 903 cells, 0.5 μM MK1775 or 1.25 μM alone or 0.63 to 2.5 μM GDC0068 alone reduced cell viability by 20 to 25% (FIG. 3A). In contrast, combinations of 0.5 μM MK1775 or 1.25 μM MK1775 with 0.63 μM GDC0068, 1.25 μM GDC0068 or 2.5 μM GDC0068 achieved larger decreases in cell viability (FIG. 3A).

In UACC 903 cells, 0.63 μM MK1775 alone or 2.5 to 10 μM AZD5363 alone reduced cell viability by 35 to 40% (FIG. 3C). In contrast, combinations of 0.63 μM MK1775 with 2.5 μM AZD5363, 5.0 μM AZD5363, 7.5 μM AZD5363 or 10 μM AZD5363 achieved larger decreases in cell viability (FIG. 3C).

FIGS. 3A and 3C show bar graphs representing significant reduction in cell viability of UACC 903 melanoma cells treated with combination of AKT inhibitors (GDC0068 or AZD-5363) and WEE1 inhibitor (MK1775). Columns; mean (n=6-8), error bars show SEM.

The calculated combination index (CI) values for results shown in FIGS. 3A and 3C are characterized by strong synergy between the AKT inhibitor and the WEE1 inhibitor, FIG. 3B and FIG. 3D.

In C8161.C19 cells, 0.15 μM MK1775 alone or 0.31 to 2.5 μM GDC0068 alone reduced cell viability (FIG. 3E). In contrast, combinations of 0.15 μM MK1775 with 0.31 μM GDC0068, 0.63 μM GDC0068, 1.25 μM GDC0068 or 2.5 μM GDC0068 achieved larger decreases in cell viability (FIG. 3E).

In C8161.C19 cells, 0.15 μM MK1775 alone or 1.25 to 10 μM AZD5363 alone reduced cell viability of the cells (FIG. 3G). In contrast, combinations of 0.15 μM MK1775 with 1.25 μM AZD5363, 2.5 μM AZD5363, 5.0 μM AZD5363 or 10 μM AZD5363 achieved larger decreases in cell viability (FIG. 3G).

FIGS. 3E and 3G show bar graphs representing significant reduction in cell viability of C8161.C19 melanoma cells treated with combinations of AKT inhibitors (GDC0068 or AZD-5363) and WEE1 inhibitor (MK1775). Columns; mean (n=6-8), error bars show SEM.

The calculated combination index (CI) values for results shown in FIGS. 3E and 3G are characterized by synergy between the AKT inhibitor and the WEE1 inhibitor, FIG. 3F and FIG. 3H.

Comparable but slightly less synergistic results were obtained with the AKT inhibitor and the WEE1 inhibitor combinations in C8161.C19 cells indicating that the identified synergy is independent from the BRAF mutational status.

In contrast to the weak activity of AZD5363 (AKT inhibitor), MK1775 (WEE1 inhibitor), was significantly more effective on the C8161.C19 cell line and necessitated significantly lower concentrations.

The synergy of GDC0068 and MK1775 was further confirmed with colony formation assay for UACC 903 cells. A significant reduction in number of colonies formed was observed following co-treatment with the two agents.

In Vivo Analysis of Synergy, Cell Proliferation and Apoptosis in Time and Size Matched Tumors

Tumor kinetics studies were undertaken in athymic-Foxn1^{nu} nude mice (Harlan Laboratories, Indianapolis, Ind., USA). In genetic knockdown models, siRNA targeting AKT3 and WEE1 alone or in combination was nucleofected into 1×10⁶ UACC 903 or 1205 Lu cells as described above. Cells transfected with scrambled siRNA were used as a control. After plating and allowing the cells to recover for 48 hours, 1×10⁶ cells were aliquoted in 0.2 ml of 10% FBS-DMEM and then injected subcutaneously above both the left and right rib cages of 4-6 week old female mice (3-4 mice/group). Dimensions of developing tumors were measured on alternate days, using calipers to estimate size by multiplying length, width and depth in mm³. For pharmacological synergy studies, 1×10⁶ UACC 903 cells were injected subcutaneously above both the left and right rib cages of 4-6 week old female mice. 5 days following cell injection, oral treatments of MK1775 (50 mg/kg, daily) and AZD5363 (150 mg/kg, daily) were initiated. Drugs were prepared in 0.5% methylcellulose, 0.5% Tween 80, 5% DMSO and 200 uL administered per oral treatment.

To generate time and size matched tumors, 1×10⁶ or 10×10⁶ of UACC 903 melanoma cells were nucleofected with siScrambled, siAKT3, siWEE1 or siAKT3+siWEE1, respectively, as detailed above and injected into nude mice. Tumors were removed from euthanized mice at days 9 and 11, flash frozen in liquid nitrogen, pulverized and protein lysates collected by the addition of 600 to 800 uL of RIPA protein lysis buffer containing Halt Protease & Phosphatase Inhibitor Cocktail (Thermo Scientific, Rockford, Ill., USA). Protein concentration was measured using the bicinchoninic acid assay kit (Thermo Scientific, Rockford, Ill., USA) followed by Western blotting to measure the levels of AKT3, WEE1 and p53 proteins in tumors. The band intensity was quantified by scanning the optical density of each band using ImageJ. Cell proliferation rates were measured using a mouse anti-human Ki-67 antibody from BD Pharmigen (BD Biosciences, San Diego, Calif.). Apoptosis rate was measured using the “terminal deoxynucleotidyl transferase-mediated dUTP nick end labeling (TUNEL)” TMR Red Apoptosis kit (Roche, Mannheim, Germany). Number of Ki-67 or TUNEL stained cells were quantified as the percentage of total cells in tumors. Sections were imaged using Nikon Eclipse 600 and quantified using IP lab imaging software (Scanalytics, Fairfax, Va.). A minimum of six different tumors with 4 to 6 fields per tumor was analyzed.

At the end of the study, blood samples were collected from each mouse, serum was separated and levels of various toxicity markers were analyzed. All of the measured biomarkers in samples collected from mice were within the normal range which excludes any toxicity caused by these drugs. Furthermore, visceral organs such as, heart, lungs, liver, intestine, spleen and kidney from each mouse were also collected, fixed in formalin and embedded in paraffin to assess any toxicity associated abnormality in cell morphology and tissue structure following hematoxylin and eosin staining.

Targeting AKT3 and WEE) Inhibited Xenografted Melanoma Tumor Growth by Decreasing Proliferation and Inducing Apoptosis in Mice

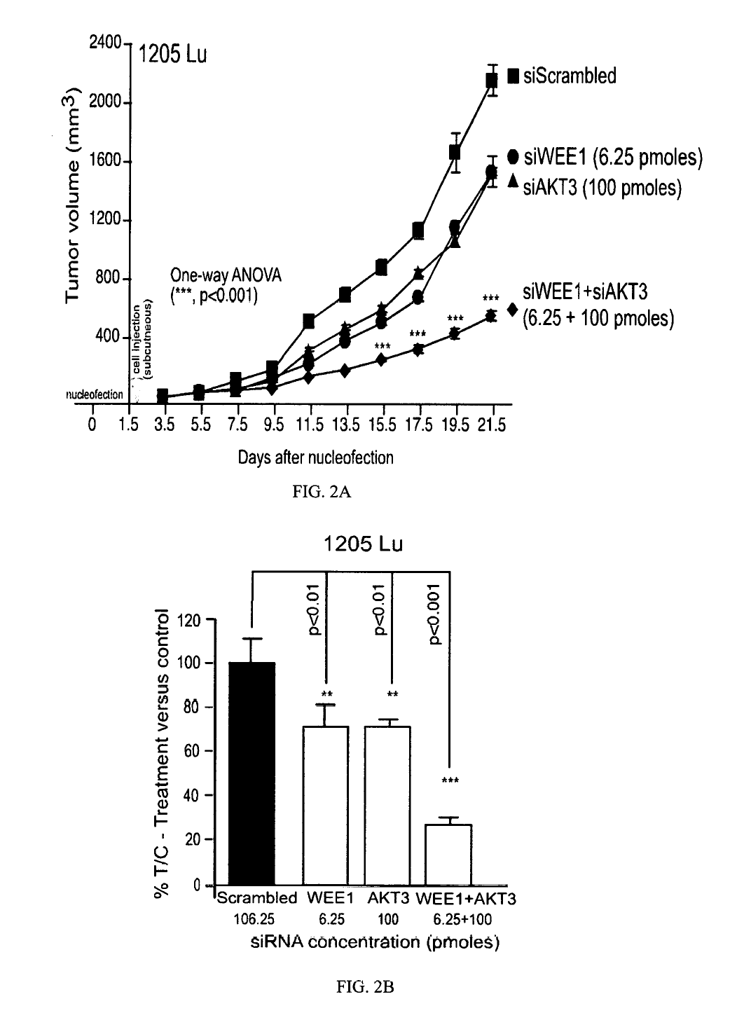

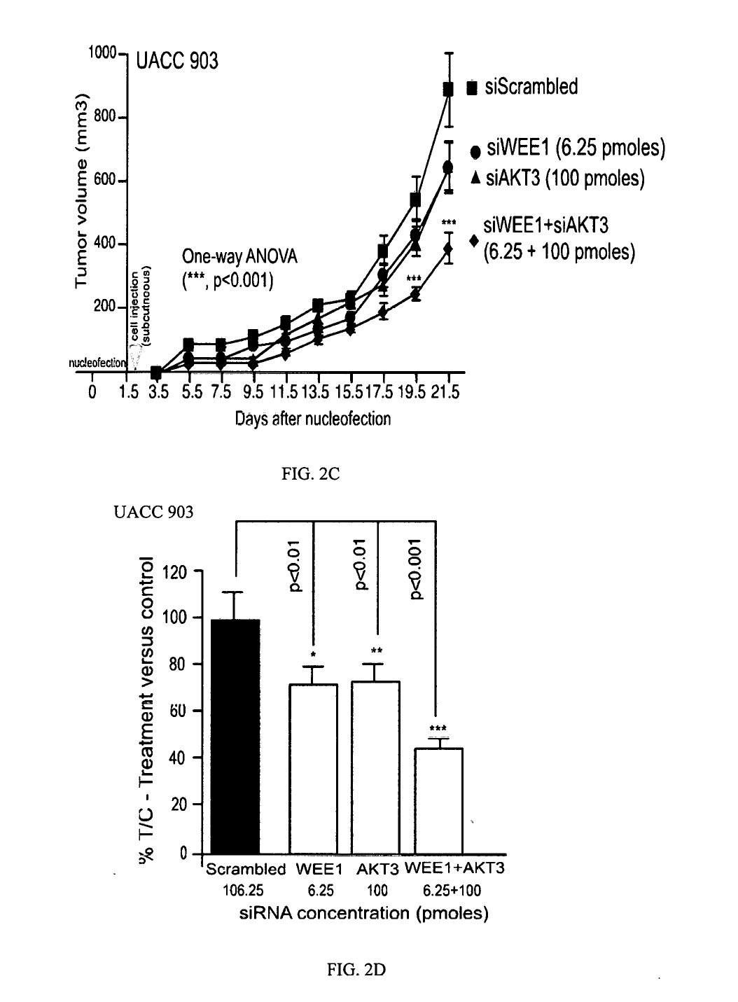

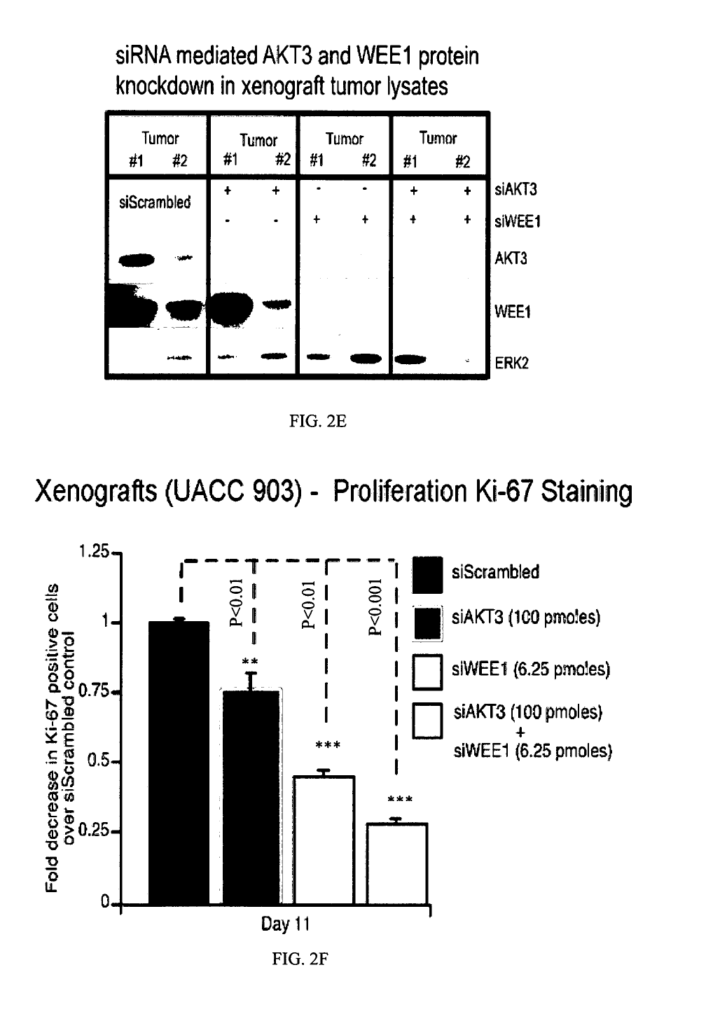

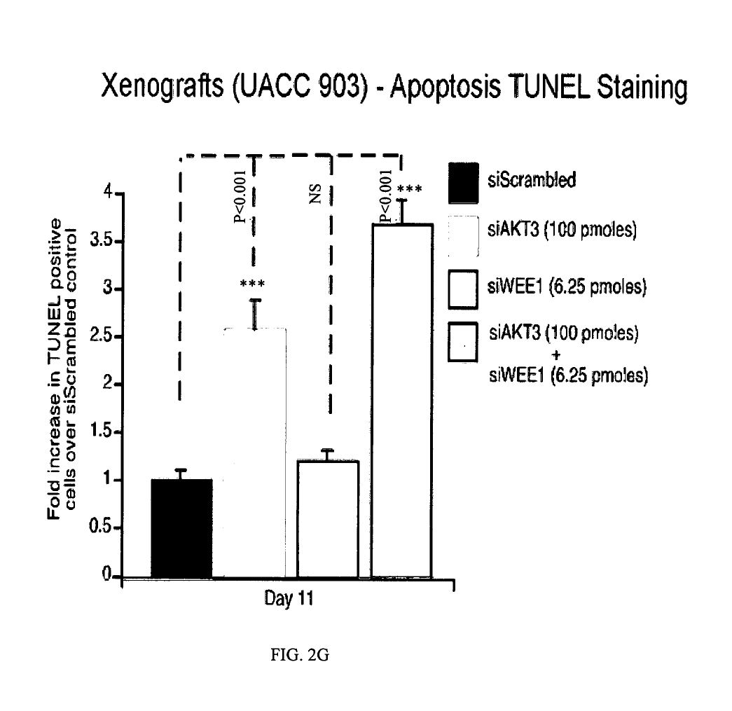

Since targeted inhibition of AKT3 and WEE1 synergistically reduced melanoma cell survival in cultured cells, efficacy of this combination was examined in human melanoma xenograft models. 1×10⁶ 1205 Lu or UACC 903 cells were transfected with siRNA targeting AKT3 (100 pmole) and WEE1 (6.25 pmole) alone or in combination, and cells transfected with scrambled siRNA (106.25 pmole) were used as a control. The concentration of WEE1 siRNA for these studies was selected based on the titration for tumor inhibition to occur in a similar range as with 100 pmole of AKT3 knockdown. This amounted to 6.25 pmole for WEE1 siRNA and 100 pmole for AKT3 siRNA (FIGS. 2A-2D). Scrambled siRNA was used to compensate for total siRNA amount in AKT3 or WEE1 alone groups. Two days after transfection, cells were subcutaneously injected into left and right flanks of 4 to 6 week old nude mice and volumes of tumors were measured on alternate days for three weeks. In both melanoma cell lines, combinatorial knockdown of AKT3 and WEE1 was significantly more effective at inhibiting tumor development in contrast to single knockdowns (FIGS. 2A-2D). A 60 to 80% decrease in tumor volume was observed when AKT3 and WEE1 were targeted together compared to a 25% decrease when each kinase was targeted alone. In FIGS. 2A and 2C, the line graph presents tumor volume (mm³). Significance was measured by the one way analysis of variance, followed by the post hoc test, ***P<0.01. Each point represents average data obtained from six nude mice. Data; means±SEM. FIGS. 2B and 2D show bar graphs representing percentage of treatment (grey and white bars) versus control (black bar) at day 21. Significance was measured by the one way analysis of variance, followed by the post hoc test, *P<0.05, **P<0.01, ***P<0.01. Columns; mean (n=6), error bars show SEM. Data were obtained from duplicate experiments with three mice per group, containing two tumors per mouse. Efficiency of protein knockdown by siRNA was measured by Western blotting of size and time matched xenografted tumor lysates. Decreased levels of AKT3 and WEE1 protein expression was observed also in tumor lysates (FIG. 2E). FIG. 2E shows siRNA-mediated knockdown of AKT3 or WEE1 protein expression in xenograft tumors. Western blot analysis shows knockdown of AKT3 and WEE1 protein levels in tumor lysates harvested at day 9. ERK2 served as a control for equal protein loading.

To dissect the cellular processes mediating enhanced tumor inhibition when targeting AKT3 and WEE1, rates of cellular proliferation and apoptosis were measured in size and time matched human melanoma xenograft tumors. Formaldehyde fixed, paraffin embedded sections of tumors were stained with Ki-67 to measure tumor cell proliferation rates and TUNEL to detect apoptotic cell death. Tumor sections were immunostained for Ki-67 or TUNEL to measure proliferation and apoptosis, respectively. Co-targeting AKT3 and WEE1 together led to 4-fold inhibition in melanoma tumor cell proliferation (FIG. 2F) and 3.5-fold increase in apoptosis (FIG. 2G). In contrast, targeting these genes alone led to 1.3 and 2 fold decrease in proliferating cells as well as 2.5 to 1.25 fold increase in apoptotic cells, respectively. Bar graphs show fold change in Ki-67 or TUNEL-positive cells from xenograft tumors of mice treated as indicated compared to scrambled siRNA control. Data were obtained from three to four tumors with four to five fields averaged per tumor. Significance measured by the one way analysis of variance, followed by the post hoc test, **P<0.01, ***P<0.001, NS; Non-significant. Columns, mean; error bars show SEM.

Inhibitors of AKT and WEE1 Kinases Synergize to Inhibit Xenografted Melanoma Tumor Growth

To assess whether pharmacological inhibitors of AKT and WEE1 kinases (MK1775 and AZD-5363) also synergistically inhibit tumor development in vivo, activity of this drug combination on xenografted UACC 903 human melanoma tumors was assessed.

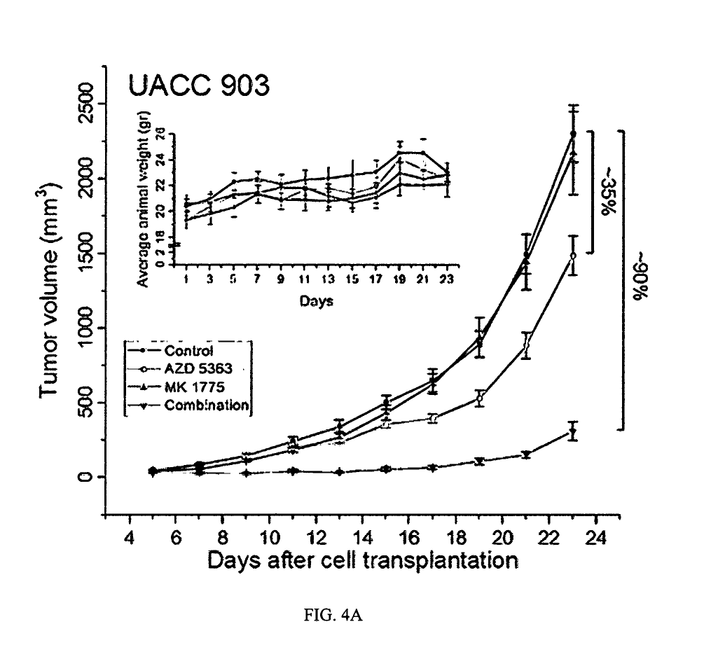

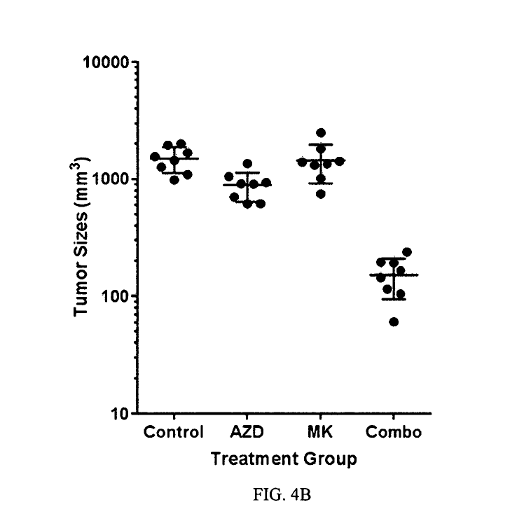

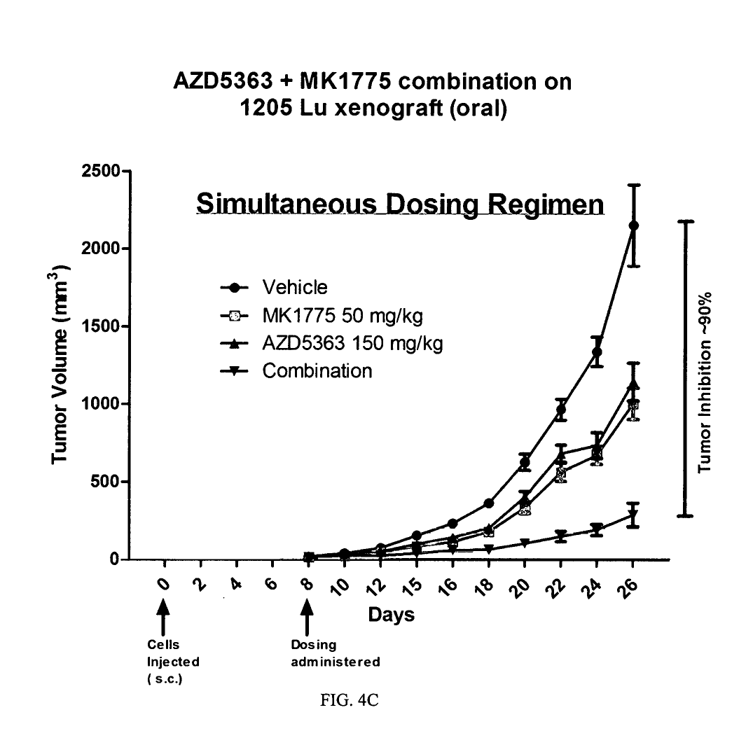

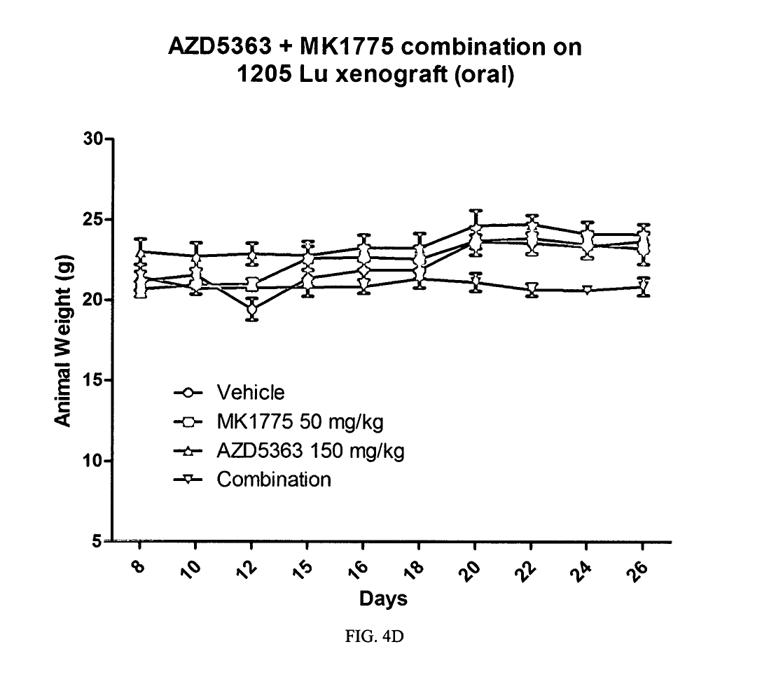

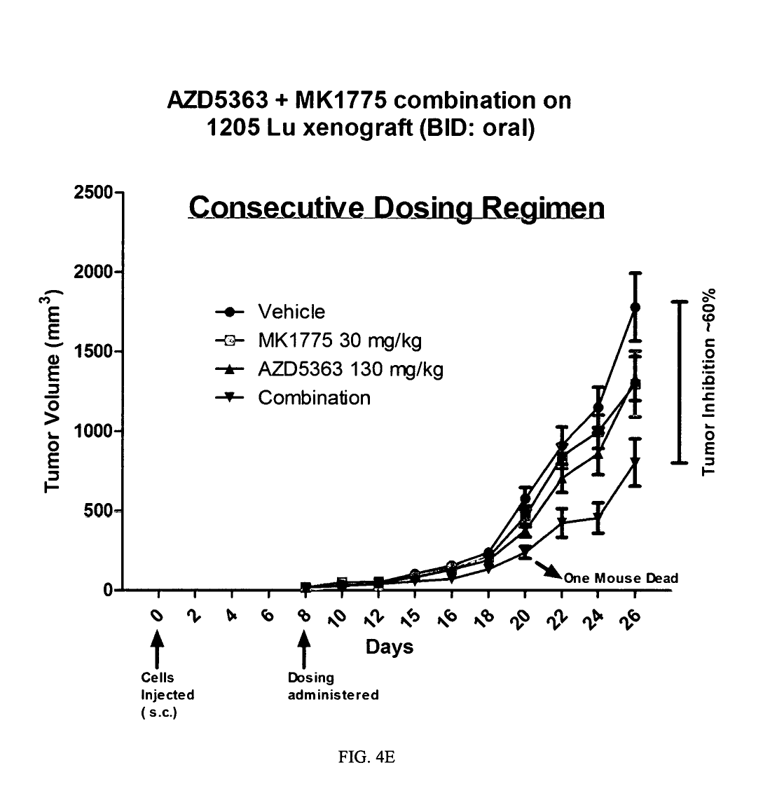

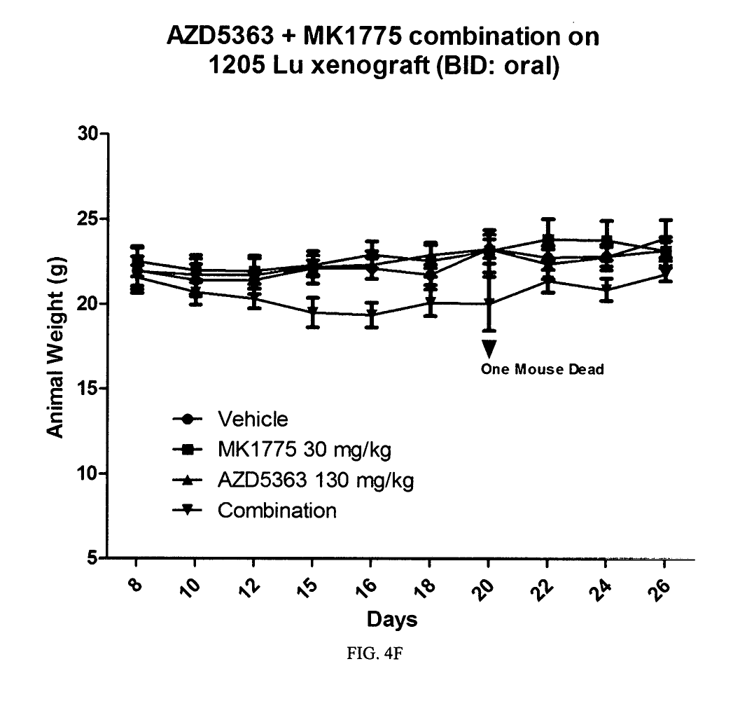

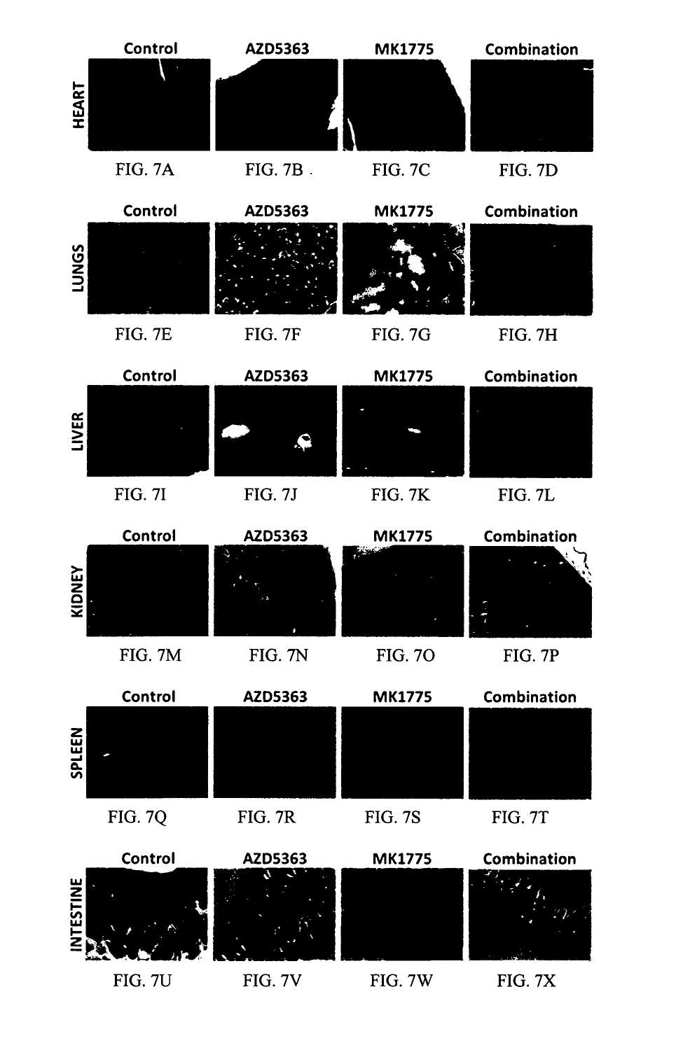

Oral administrations of MK1775 (50 mg/kg), AZD5363 (150 mg/kg) or a combination of MK1775 (50 mg/kg) and AZD5363 (150 mg/kg) were initiated when a vasculated xenograft tumor had formed and the oral administrations continued daily for 18 days. MK1775 alone was ineffective at reducing UACC 903 tumor development while AZD5363 alone led to a 36% decrease in tumor size (FIG. 4A, error bars show ±SEM). In contrast, the combination of the two agents was strongly synergistic against the xenografts of both cell lines, reducing tumor development by >90% compared to vehicle treatments (FIGS. 4A and 4B). No significant changes were observed in animal body weights or blood biomarkers indicative of vital organ function (FIG. 4A inset, error bars show ±SEM; and Table III). Histologic examination of major organs at the end of treatments showed no obvious change in the cellular morphology or tissue architecture suggesting no toxicity associated with the performed treatments (FIGS. 7A-7X).

TABLE III
Serum analysis

| | | Reference | | | | | |
|---|---|---|---|---|---|---|---|
| | | Levels | | Control | AZD5363 | MK1775 | Combination |
| Liver | SGPT | 33-132 | u/L | 42.5 ± 2.2 | 41.3 ± 7.4 | 55 ± 10.1 | 46 ± 4.2 |
| Function | (ALT) | | | | | | |
| | ALKP | 62-209 | u/L | 79 ± 6.9 | 86.3 ± 7.5 | 75 ± 5.6 | 102.7 ± 9.5 |
| | ALB | 2.5-4.8 | mg/dL | 2.5 ± 0.1 | 2.5 ± 0 | 2.6 ± 0.1 | 2.6 ± 0 |
| | TBIL | 0.1-0.9 | mg/dL | 0.6 ± 0.2 | 0.5 ± 0.1 | 0.6 ± 0 | 0.6 ± 0.1 |
| Kidney | BUN | 18-33.7 | mg/dL | 20 ± 2.7 | 25.8 ± 2.2 | 25.3 ± 1.9 | 20.7 ± 1.5 |
| | CREA | 0.1-0.3 | mg/dL | 0.1 ± 0 | 0.1 ± 0 | 0.1 ± 0 | 0 ± 0 |
| Muscle | GLOB | g/dL | | 2.8 ± 0.1 | 2.6 ± 0.1 | 2.6 ± 0 | 2.5 ± 0.1 |
| Pancreas | AMYL | 1691-3615 | U/L | 1830 ± 66.7 | 1891 ± 129.7 | 2251.8 ± 50.2 | 1966.3 ± 134.8 |
| Others | CHOL | 92-157 | mg/dL | 116 ± 5.3 | 113.5 ± 7.6 | 142.5 ± 4.6 | 132.7 ± 7.8 |
| | GLU | 198-247 | mg/dL | 191.8 ± 11.7 | 214 ± 30.6 | 237.5 ± 31.4 | 238.3 ± 47.2 |
| | TP | 3.6-6.6 | g/dL | 5.3 ± 0.2 | 5.1 ± 0.1 | 5.2 ± 0.1 | 5.1 ± 0.1 |

To further assess whether pharmacological inhibitors of AKT and WEE1 would also synergistically inhibit tumor development in vivo, the efficacy of AZD5363 and MK1775 alone and in combination was evaluated on 1205 Lu melanoma xenografts in mice. Oral administrations of AZD5363 alone, MK1775 alone, AZD5363 and MK1775 administered concurrently or sequentially, or control materials were initiated when a vasculated tumor had formed and continued for 18 days according to the schedule outlined below. 1205 Lu melanoma xenograft tumors were established by subcutaneous injection of 1×10⁶ 1205 Lu cells injected above both left and right rib cages of 4-6 week-old female Athymic-Foxn1nu nude mice (Harlan Sprague Dawley). Six days later, when a fully vascularized tumor had formed, mice were randomly divided into treatment groups described below. Body weight in grams and dimensions of developing tumors in mm³ were measured on alternate days (1-6). Drugs were prepared in 0.5% methylcellulose, 0.5% Tween 80, 5% DMSO and 200 μL administered per oral treatment.

AZD5363 and/or MK1775, or control materials, were orally administered to groups of tumor bearing mice as follows:

Group 1 (vehicle control, 0.5% methylcellulose, 0.5% Tween 80, 5% DMSO) daily for 18 days;

Group 2 (AZD5363, 150 mg/kg) daily for 18 days;

Group 3 (MK1775, 50 mg/kg) daily for 18 days; and

Group 4 (AZD5363, 150 mg/kg+MK1775, 50 mg/kg) daily for 18 days.

Group 5 (vehicle control, 0.5% methylcellulose, 0.5% Tween 80, 5% DMSO) b.i.d. (twice a day) for 18 days;

Group 6 (AZD5363, 130 mg/kg b.i.d. for 4 days on, 3 days off) for 18 days;

Group 7, (MK1775, 30 mg/kg b.i.d. for 3 days on, 4 days off) for 18 days; and

Group 8 (AZD5363, 130 mg/kg b.i.d. for 4 days and 3 days of 30 mg/kg bid of AZD1775) for 18 days.

Results of Daily Treatment with AZD5363 and/or MK1775

Treatment with AZD5363 or MK1775 alone led to ˜50% reduced growth of 1205 Lu xenograft compared to the control (FIG. 4C). In contrast, administration of the combination of AZD5363 and MK1775 was strongly synergistic, reducing tumor development by ˜90% compared to controls (FIG. 4C). No significant changes were observed in animal body weights suggesting no obvious toxicity associated with major organ function (FIG. 4D).

Results of Consecutive Alternating Treatments

As noted above, for this example, AZD5363 was administered orally at 130 mg/kg bid (twice a day) for 4 days on, 3 days off and this pattern was continued for 18 days; or AZD1775 was administered orally at 30 mg/kg bid for 3 days on, 4 days off and this pattern was continued for 18 days; or AZD1775 was administered orally at 30 mg/kg bid for 3 days on (i.e. the three days on which AZD5363 was not administered), 4 days off (i.e. the four days when AZD5363 was administered) and this pattern was continued for 18 days, for separate groups of tumor bearing mice. AZD5363 or MK1775 alone (Groups 6 and 7) led to 30% reduced growth of 1205 Lu xenograft tumors compared to the control (FIG. 4E). In contrast to the simultaneous drug dosing regimen (Group 4), the consecutive dosing of both AZD5363 and MK1775 (Group 8) led to a moderate reduction in tumor growth of ˜60% compared to the control (FIG. 4E). Approximately 20% body weight loss was observed shortly after treatment in the combination group (Group 8) followed by death of a mouse at day 20, suggesting some dose related toxicity (FIG. 4F).

Inhibition of both AKT and WEE1 synergistically inhibits viability of MCF7 human breast cancer cells and PC-3 human prostate cancer cells.

WEE1 inhibitor MK1775 and AKT inhibitor AZD5363 were dissolved in DMSO. Cytotoxicity of these agents alone or in combination was measured by plating 2.5 to 10×10′ cells into 96-well plates followed by growth for 24 to 48 hours in a humidified 37° C. cell culture incubator. The cells were treated for 72 hours at concentrations of WEE1 inhibitor MK1775, AKT inhibitor AZD5363, or both, at doses ranging from 0.15 to 15 micromolar. Cytotoxicity was measured using the CellTiter 96 Aqueous Non-Radioactive Cell Proliferation Assay Kit (Promega). The CI values were calculated using CalcuSyn software.

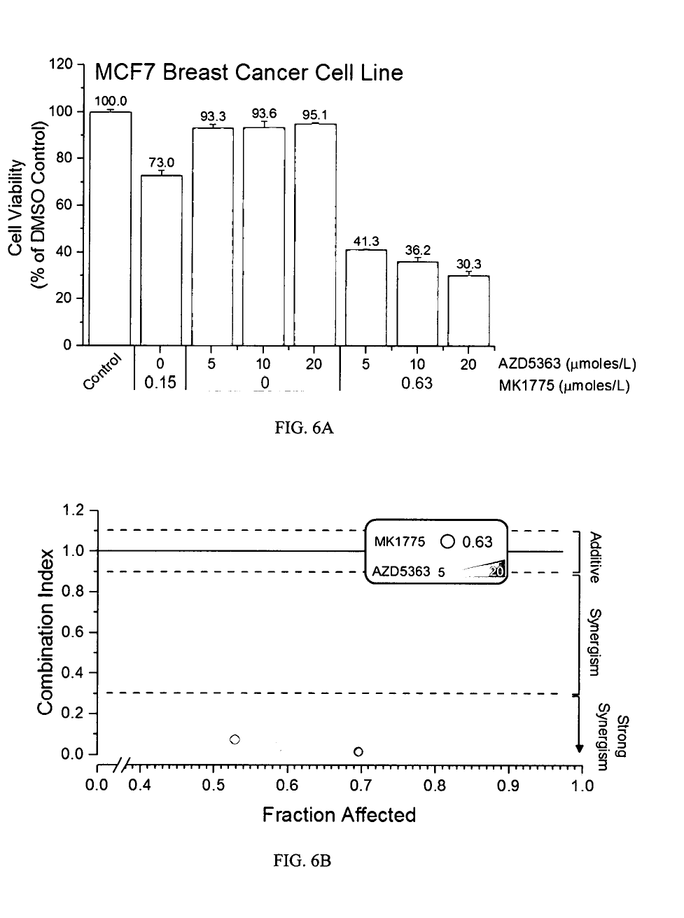

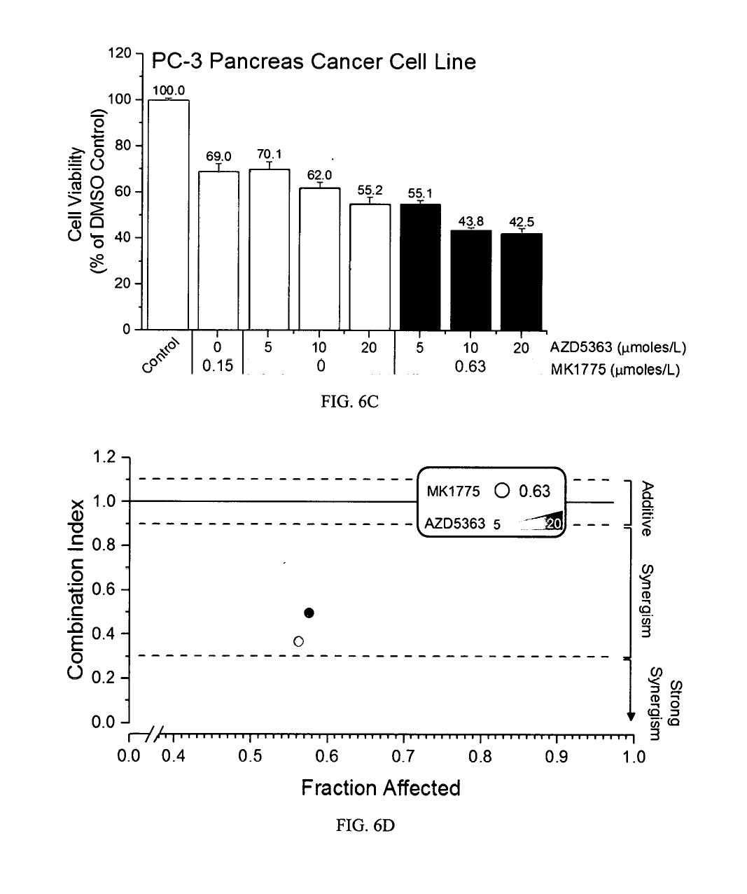

FIG. 6A shows the effect of an AKT inhibitor (5, 10 or 20 micromolar AZD5363) alone, a WEE1 inhibitor (0.63 micromolar MK1775) alone or combinations of an AKT inhibitor and a WEE1 inhibitor (5 micromolar AZD5363 and 0.63 micromolar MK1775, 10 micromolar AZD5363 and 0.63 micromolar MK1775 or 20 micromolar AZD5363 and 0.63 micromolar MK1775) on human MCF-7 breast cancer cells in vitro.

FIG. 6B shows results of Chou-Talalay analysis for determining the combination index of the treatment of human MCF-7 breast cancer cells with combinations of an AKT inhibitor and a WEE1 inhibitor (5 micromolar AZD5363 and 0.63 micromolar MK1775 shown as lightest circle, 10 micromolar AZD5363 and 0.63 micromolar MK1775, shown as medium intensity gray circle or 20 micromolar AZD5363 and 0.63 micromolar MK1775, shown as darkest gray circle, on human MCF-7 breast cancer cells in vitro.

FIG. 6C shows the effect of an AKT inhibitor (5, 10 or 20 micromolar AZD5363) alone, a WEE1 inhibitor (0.63 micromolar MK1775) alone or combinations of an AKT inhibitor and a WEE1 inhibitor (5 micromolar AZD5363 and 0.63 micromolar MK1775, 10 micromolar AZD5363 and 0.63 micromolar MK1775 or 20 micromolar AZD5363 and 0.63 micromolar MK1775) on human PC-3 prostate cancer cells in vitro.

FIG. 6D shows results of Chou-Talalay analysis for determining the combination index of the treatment of human MCF-7 breast cancer cells with combinations of an AKT inhibitor and a WEE1 inhibitor (5 micromolar AZD5363 and 0.63 micromolar MK1775 shown as lightest circle, 10 micromolar AZD5363 and 0.63 micromolar MK1775, shown as medium intensity gray circle or 20 micromolar AZD5363 and 0.63 micromolar MK1775, shown as darkest gray circle, on human PC-3 prostate cancer cells in vitro.

Additional cancer cell types were assayed for the effect of inhibition of both AKT and WEE1 on cell proliferation. For these assays, WEE1 inhibitor MK1775 and AKT inhibitor AZD5363 were dissolved in DMSO. Cytotoxicity of these agents alone or in combination was measured by plating 2.5 to 10×10³ cells of the type indicated in Table IV into 96-well plates followed by growth for 24 to 48 hours in a humidified 37° C. cell culture incubator. The cells were treated for 72 hours at concentrations of WEE1 inhibitor MK1775, AKT inhibitor AZD5363, or both, at doses ranging from 0.31 to 10 micromolar. Cytotoxicity was measured using the CellTiter 96 Aqueous Non-Radioactive Cell Proliferation Assay Kit (Promega). The CI values were calculated using CalcuSyn software

TABLE IV

| | | AZD5363 + MK1775 | |
|---|---|---|---|
| | Representative | Identified Synergism | |
| Cancer type | Cell line | CI Values | Effect |
| Melanoma | UACC 903 | 0.098 | Synergy |
| Sarcoma | HT1080 | 0.034 | Synergy |
| Breast | T47D | 0.221 | Synergy |
| Glioblastoma | U87MG | 0.335 | Synergy |
| Prostate | LNCAP | 0.153 | Synergy |
| T-cell acute | Nalm6 | 0.264 | Synergy |
| lymphoblastic | | | |
| leukaemia (T-ALL) | | | |
| Colon | Caco2 | 0.934 | Additive |
| Lung | A549 | 1.042 | Additive |
| Liver | HepG2 | 0.984 | Additive |

The CI values in Table IV denote the best value obtained for each cell line.

A synergistic effect of inhibition of both AKT3 and WEE1 kinases was observed in cell types characterized by high gene expression levels of at least one of AKT3 or WEE1.

TABLE V
Gene expression levels of AKT and WEE1

| Cancer | AKT Expression | WEE1 Expression |
|---|---|---|
| Melanoma | High | Low |
| Sarcoma | High | Medium |
| Breast | Low | High |
| Glioblastoma | Medium | High |
| Prostate | High | Low |
| T-ALL | Medium | High |
| Colon | Low | Low |
| Lung | Low | Low |
| Liver | Low | Medium |

High, Medium and Low AKT expression or WEE1 expression in Table V represent the level of expression of genes in specific cancers when normalized to their expression in all the cancer types as presented in The Cancer Genome Atlas (TCGA), a publicly available database maintained by the National Institutes of Health, U.S.

Co-Targeting AKT3 and WEE1 Results in a Unique Mechanism of Synergistically Inhibiting Melanoma Tumor Development

To identify the synergistic mechanisms through which co-targeting AKT3 and WEE1 inhibits melanoma tumor development, first, alterations in gene expression levels were determined in genome-wide level through mRNA sequencing of UACC 903 cells following siRNA or pharmacological targeting of AKT and WEE1 kinases. Next, reverse phase protein arrays were used to validate alterations at the protein level, which was subsequently further validated through Western blotting.

RNA Sequencing

Twenty-four hours following treatment with tested agents, total RNA was extracted from UACC 903 cells using mirVana RNA isolation kit (Life Technologies, Grand Island, N.Y.). Next, using SureSelect Strand Specific RNA Library Preparation kit (Agilent) cDNA libraries were constructed, polyA RNA was purified with oligo (dT) beads and then extracted RNA was processed through fragmentation, reverse transcription, end repair, 3′-end adenylation, adaptor ligation, PCR amplification and SPRI bead purification (Beckman Coulter). For multiplexed high-throughput sequencing, the unique barcode sequences were incorporated. Libraries were then denatured using the Illumina protocol, diluted with pre-chilled hybridization buffer and loaded onto TruSeq SR v3 flow cells on an Illumina HiSeq 2500 (50 cycles, single-read). Sequencing reads were extracted by using Illumina CASAVA pipeline Version 1.8 and aligned to the human reference genome (hg38) using Tophat (version 2.0.9) as described in detail in Trapnell et al, Bioinformatics 2009; 25(9): 1105-11. SeqMonk (version 0.32.0) was used for the identification of differentially expressed genes. Following RNA-Seq quantitation, 75% percentile normalization was performed and significant alterations were determined via intensity difference test with Benjamini and Hochberg multiple testing correction (p-value <0.05). Enrichment analyses were performed using Enrichr online gene set enrichment analysis tool described in Chen et al., BMC Bioinformatics, 14:128, 2013.

RNA Sequencing identified 41 significantly deregulated genes following siRNA-mediated knockdown of WEE1 in UACC 903 cells. Enrichment analyses of the 22 upregulated genes showed the p53 protein as a significantly enriched transcriptional regulator. As a matter of fact, 14 of the 22 genes were modulated by p53 gene family transcription factors. Analysis of the remaining 19 downregulated genes identified transcription factors FOXM1 and E2F4 as prominent modulators of this effect. 13 of the 19 (70%) downregulated genes were modulated by these two transcription factors. Enrichment analysis of all 41 deregulated genes implicated induction of DNA damage and cell cycle deregulation. These observations were consistent with the function of WEE1, since, as a regulator of the G2/M phase checkpoint, inhibition of WEE1 has been shown to induce DNA damage through perturbation of the cell cycle.

siRNA mediated knockdown of AKT3 only altered 18 genes limiting the functionality of the enrichment analysis. However, of these 18 genes, downregulation of CDK6 was the most prominent as it is an important effector of AKT signaling. In contrast to targeting AKT3 alone, siRNA mediated co-inhibition of WEE1 and AKT3 kinases deregulated 40 genes, of which 14 were upregulated and 26 were downregulated. PLK1 signaling modulating the FOXM1 transcription factor was enriched only during the combination treatment. FOXM1 and E2F transcription factors were enriched among the downregulated genes while GATA3 was enriched among the upregulated ones.

Treatment of UACC 903 cells with pharmacological agents targeting AKT3 and WEE1 kinases led to similar results to those observed following siRNA mediated targeting and knockdown of these genes.

WEE1 inhibitor, MK1775, led to deregulated expression of 94 genes.

Similar to the genetic knockdown of WEE1, FOXM1/E2F4 transcription factors were enriched among the downregulated genes, and the p53 transcription factor was enriched among the upregulated ones. 38 of the 57 upregulated genes were transcriptionally regulated by the p53 transcription factor family. The AKT inhibitor, AZD-5363, deregulated 64 genes. Downregulation of the cell cycle regulator CDC25A was a notable alteration, but like siRNA targeting of AKT3, enrichment analyses did not implicate any particular pathway. In contrast, the combination of AZD536 with MK1775 altered expression of 84 genes. 42 of these genes were unique to the combination treatment. Among the promoters of “unique” gene list, FOXM/E2F4 transcription factors were significantly enriched, as 22 of the 24 downregulated genes were modulated by these two transcription factors. Similar to the siRNA-mediated cotargeting of AKT3 and WEE1, decreased PLK1 levels modulating FOXM1/E2F signaling was notable following the combination of MK1775 and AZD5363. Thus, several key proteins involved in the regulation of cell cycle (e.g., PLK1, FOXM1, E2F1) and DNA damage signaling (e.g., TP53INP1 and TP5313) were synergistically deregulated following MK1775 and AZD5363 treatments.

To validate the signaling alterations at the protein level, Reverse Phase Protein Arrays (RPPA) were used.

UACC 903 cells were transfected with siRNAs targeting AKT3 (siAKT3, 50 pmole), WEE1 (siWEE1, 25 pmole) or a combination thereof. siScramble (50 pmole) was used as a control. 48 hours after transfections, cell lysates were collected using protein extraction reagent (T-PER, Thermo Scientific) supplemented with 1 mM EDTA, 5 mM NaF, 2 μM staurosporine, PhosSTOP Phosphatase Inhibitor Cocktail (Roche), and Complete Mini Protease Inhibitor Cocktail (Roche). Total protein concentration was determined by bicinchoninic acid assay (Thermo Scientific) and submitted to the Functional Proteomics Core Facility at MD Anderson for the RPPA analysis. The array was consisted of 287 antibodies including 64 phospho-specific antibodies.

Reverse Phase Protein Array analysis of UACC 903 cells transfected with siRNAs targeting AKT3 (100 pmole), WEE1 (12.5 pmole), their combination or Scrambled controls showed that AKT1/2/3 levels, pPRAS and XIAP protein levels were decreased with siAKT3.

Confirming the RNA Sequencing results, knockdown of WEE1 enhanced the expression of DNA damage marker phospho H2AX, as well as p53 and downstream p21 levels. Furthermore, FOXM1 levels were decreased concomitantly with PLK1 levels. Knockdown of AKT3 increased p21 and phospho H2AX levels while reducing total AKT, pPRAS40, XIAP, PLK1, FOXM1 and phospho-Rb levels. Targeting the two kinases synergistically increased GATA3, phospho H2AX, p53 and downstream p21 levels while leading to a significant reduction in PLK1, FOXM1, Cyclin B1 and phospho-Rb levels. These observations were in concordance with the RNA Sequencing analysis suggesting that during drug combination treatment, WEE1 inhibition leads to DNA damage that activates p53/p21 signaling, while AKT inhibition modulates this process through the PLK1/FOXM1 axes.

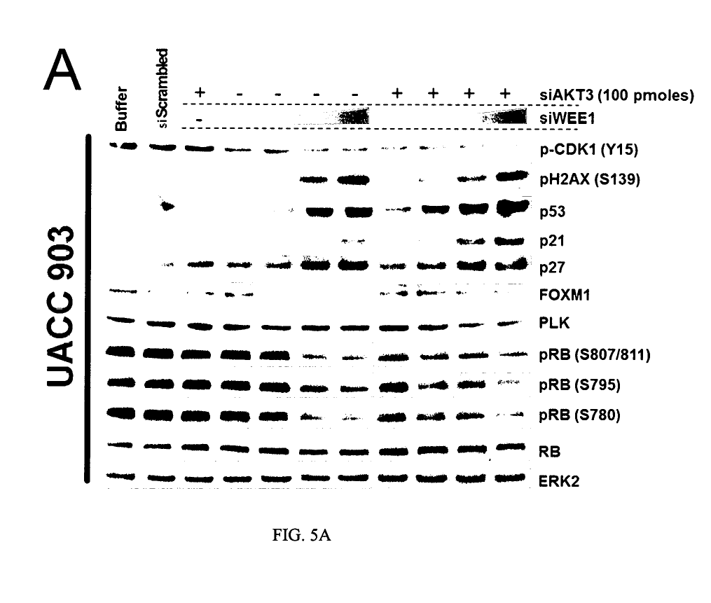

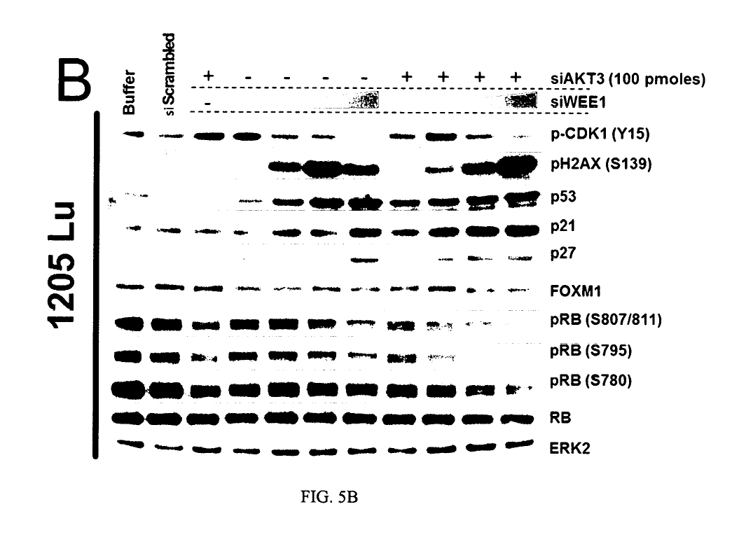

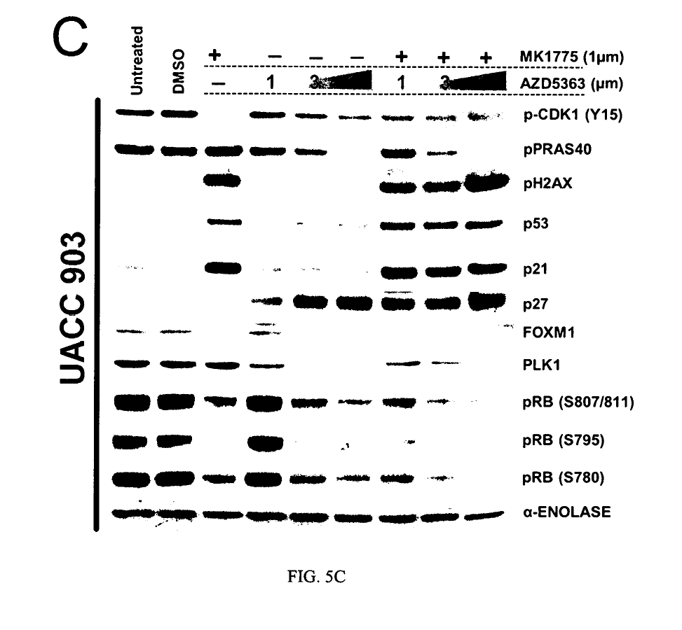

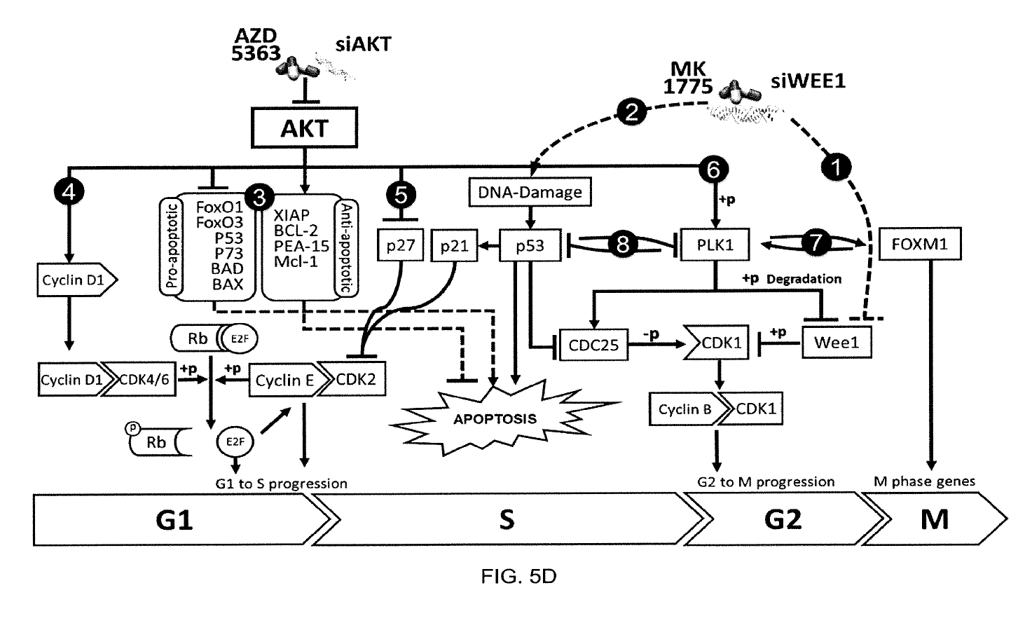

The results obtained from RPPA analyses were confirmed by Western blotting studies, results of which are shown in FIGS. 5A-C. Knockdown of FEE kinase led to a dose-dependent decrease in the phosphorylation of its substrate CDK1, FIGS. 5A and 5B. Decreased phosphorylation of CDK1 was predicted to increase its activity leading to the bypass of the G2/M checkpoint and induction of DNA damage. As expected, a dose-dependent increase in the phosphorylation of H2AX was observed. Consequently, this resulted in increased p53, p21 as well as p27 levels, which are known to be inhibitory to cell proliferation, FIGS. 5A, B, and D. FOXM1 and phosphorylated RB levels were also dose-dependently decreased by WEE1 knockdown, FIG. 5A. Decreased phosphorylation of RB and enhanced expression of p21 were observed with the AKT3 knockdown. Co-targeting of AKT3 and WEE) was more effective at reducing FOXM1 and phosphorylated RBI levels while enhancing cellular levels of p53, FIGS. 5A and 5B. Moreover, consistent with RNA sequencing and RPPA experiments, PLK1 levels were synergistically downregulated when targeting AKT3 and WEE1, FIGS. 5A and 5C.

Pharmacological inhibition of AKT and WEE1 kinases using AZD5363 and MK1775 treatments showed comparable results, FIG. 5C. WEE1 inhibitor, MK1775 decreased phosphorylated CDK1 levels, and AKT inhibitor AZD5363 dose-dependently decreased phosphorylated PRAS40 levels, validating inhibition of WEE1 and AKT kinases. Interestingly, AZD5363 also reduced the phosphorylation of CDK1 in a dose-dependent manner. Similar to genetic targeting, pharmacological inhibitors increased p27, p53 and p21 levels with a concomitant increase in DNA damage marker phospho-Histone H2AX, FIG. 5C. A concomitant decrease in PLK1, FOXM1, and phospho-Rb levels was observed, FIG. 5C.

FIG. 5D is a diagram showing the mechanism of synergism for co-targeting AKT and WEE1 signaling pathways. Genetic or pharmacological inhibition of WEE1 (1) suppresses inhibitory phosphorylation of CDK1 leading to early-G2/M progression. This leads DNA damage (2) and activates p53 signaling. p53 inhibits cell cycle progression by induction of p21, allowing DNA damage repair. If the DNA damage is not repairable, p53 induces apoptosis. However, in many cancer cells, apoptotic cascades are suppressed by oncogenic alterations. Over-activated AKT inhibits pro-apoptotic factors while inducing antiapoptotic factors (3). AKT signaling also enhances cell cycle progression by CyclinD1 mediated phosphorylation of RB (4) and inhibition of p27 (5). Furthermore, AKT phosphorylates and induces Polo-like kinase 1 (PLK1) (6), which in turn inhibits pro-apoptotic functions of p53 and its family members, p63 and p73 (7). In addition, PLK1 also induces FOXM1 activity and M-phase progression (8).

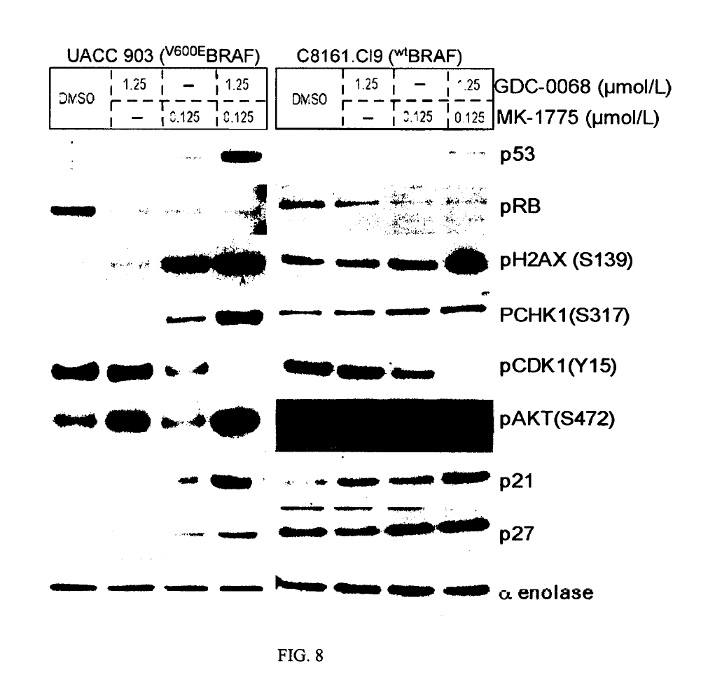

Similar results were also observed with combination of MK1775 and pan-AKT inhibitor, GDC0068, in both UACC903 and C8161.C19 cells, FIG. 8. FIG. 8 is Western blot showing MK1775 or GDC0068 induced alterations in levels of Histone H2A.X, p53, CHK1, p21, p27, phosphorylation of CDK1 and AKT, and serine phosphorylation of RBI proteins. Alpha enolase served as a control for equal protein loading.

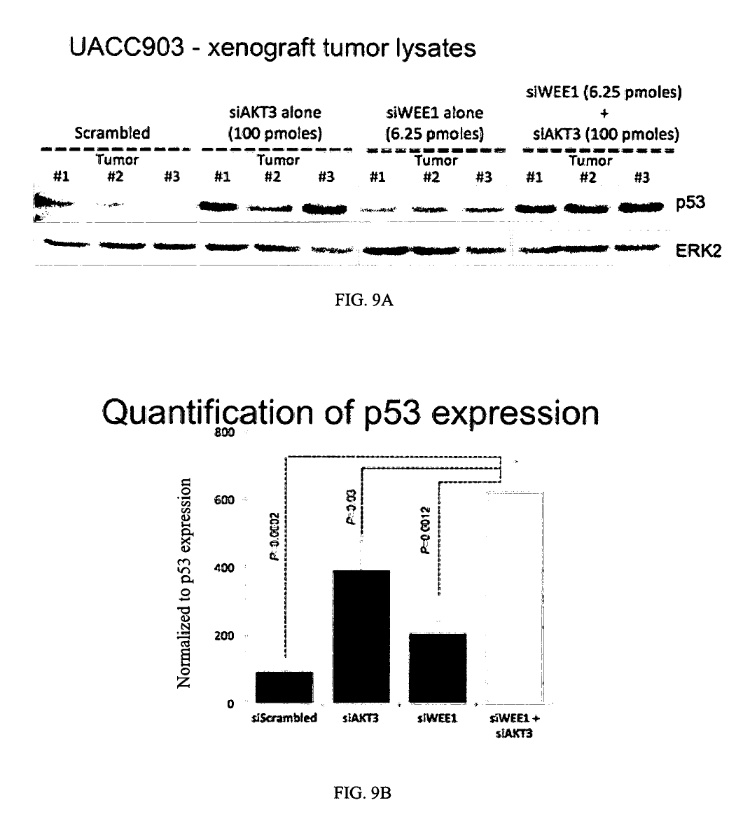

Similar to results in cultured cells, 7.5 fold higher p53 levels were observed in tumor lysates from time and size matched xenografted tumors in which AKT3 and WEE1 was co-targeted, FIGS. 9A and 9B. FIG. 9A shows Western blots of tumor lysates from UACC 903 xenografts following oral administration of AZD5363, MK1775 or their combination. Western blotting in FIG. 9A shows p53 expression in lysates from day 11 UACC 903 xenograft tumors. FIG. 9B is a bar graph representing quantification of Western blotting normalized to p53 expression at day 11. Significance measured by the t test, *P<0.05, **P<0.01, ***P<0.01. Columns, mean (n=3); error bars show SEM. ERK2 served as a control for equal protein loading.

Knockdown of WEE1 dose dependently reduced phosphorylated RBI levels (FIGS. 5A-SC). Therefore, targeting WEE1 has the ability to induce cell cycle arrest and apoptosis through induction of p53 activity.

Targeting AKT3 primarily induced apoptosis in melanoma tumors.

AKT knockdown interferes with WEE1 mediated regulation of cell cycle by controlling Polo like kinase-1 (PLK1) and consequently FOXM1 levels (FIG. 5D). The diagram in FIG. 5D shows the mechanism of synergism for co-targeting AKT and WEE1 signaling pathways. RNA-sequencing experiments showed that co-targeting AKT and WEE1 synergistically inhibits PLK1 and FOXM1 levels. RPPA array analyses and Western blot experiments have confirmed these results.

Combined inhibition of the AKT3 and WEE1 kinases synergistically reduced cellular proliferation and induced apoptosis leading to enhanced tumor inhibition. In both UACC 903 and 1205 Lu melanoma cell lines, this combination synergistically decreased phosphorylated RBI levels while increasing p53 levels, indicating a potential mechanism for the observed synergistic decrease in cell proliferation, FIGS. 5A-5C.

Both colony formation and cell viability assays showed that combination of WEE1 inhibitor, MK1775 with AKT inhibitors, GDC0068 and AZD5363, synergistically inhibits melanoma cell viability.

Targeting AKT3 and WEE1 synergistically kills melanoma cells by altering cell cycle and DNA damage signaling, enhancing the efficacy of targeting AKT3. A screening is conducted where AKT3 was targeted together with a panel of kinases identified to be important in melanoma development. siRNA-mediated knockdown of AKT3 with WEE1 synergistically inhibited viability of cultured melanoma cells, which was further validated with pharmacological inhibitors of AKT and WEE1. siRNA mediated knockdown of these two kinases led to an 75% decrease in tumor development, compared to the 25% inhibition when targeting either protein alone. Combination of AKT inhibitor, AZD5363, with WEE1 inhibitor, MK1775, led to significant synergism both in cultured cells and in animals. In xenografted melanoma tumor models, these agents had a significant anti-tumor effect, almost eliminating the tumor development. The synergistic effect of targeting AKT and WEE1 was mediated by activating p53-p21 signaling while decreasing the activity of FOXM1 and phospho RB pathways.

Items

Item 1. A composition comprising: an AKT inhibitor and a WEE1 inhibitor.

Item 2. The composition of item 1, wherein the AKT inhibitor is selected from the group consisting of: AZD5363, GDC0068, a combination of AZD5363 and GDC0068, a pharmaceutically acceptable salt, hydrate, amide or ester of any thereof, and an siRNA directed to AKT.

Item 3. The composition of item 1 or 2, wherein the WEE1 inhibitor is selected from the group consisting of: MK1775, a pharmaceutically acceptable salt, hydrate, amide or ester thereof, and an siRNA directed to WEE1.

Item 4. The composition of any of items 1-3, further comprising a pharmaceutically acceptable carrier.

Item 5. A commercial package comprising an AKT inhibitor and a WEE1 inhibitor.

Item 6. The commercial package of item 5, wherein the AKT inhibitor is selected from the group consisting of: AZD5363, GDC0068, a combination of AZD5363 and GDC0068, a pharmaceutically acceptable salt, hydrate, amide or ester of any thereof, and an siRNA directed to AKT.

Item 7. The commercial package of item 5 or 6, wherein the WEE1 inhibitor is selected from the group consisting of: MK1775, a pharmaceutically acceptable salt, hydrate, amide or ester thereof, and an siRNA directed to WEE1.

Item 8. The commercial package of any of items 5-7, wherein the AKT inhibitor and the WEE1 inhibitor are provided as a single pharmaceutical formulation.

Item 9. The commercial package of any of items 5-7, wherein the AKT inhibitor and the WEE1 inhibitor are provided as separate pharmaceutical formulations.

Item 10. A method of treating cancer in a subject in need thereof comprising:

administering a combination of an AKT inhibitor and a WEE1 inhibitor as a combination formulation or separately.

Item 11. The method of item 10, wherein administration of the combination provides a synergistic effect.

Item 12. The method of treating cancer of item 10 or 11, wherein the AKT inhibitor is selected from the group consisting of: AZD5363, GDC0068, a combination of AZD5363 and GDC0068, a pharmaceutically acceptable salt, hydrate, amide or ester of any thereof and an siRNA directed to AKT.

Item 13. The method of any of items 10-12, wherein the WEE1 inhibitor is selected from the group consisting of: MK1775, a pharmaceutically acceptable salt, hydrate, amide or ester thereof and an siRNA directed to WEE1.

Item 14. The method of treating cancer of any of items 10-13, wherein the cancer is characterized by constitutive activation of a mitogen-activated protein kinase-signaling pathway.

Item 15. The method of treating cancer of any of items 10-14, wherein the cancer is characterized by constitutive activation of a mitogen-activated protein kinase-signaling pathway associated with one or more mutations in BRAF, KIT and/or RAS.

Item 16. The method of treating cancer of any of items 10-15, wherein the cancer is characterized by constitutive activation of a mitogen-activated protein kinase-signaling pathway associated with ^{V600E} BRAF.

Item 17. The method of treating cancer of any of items 10-16, wherein the cancer is characterized by AKT dysregulation.

Item 18. The method of treating cancer of any of items 10-17, wherein the cancer is selected from the group consisting of: melanoma, colorectal cancer, thyroid cancer, breast cancer, prostate cancer, sarcoma, glioblastoma, T-cell acute lymphoblastic leukaemia, lung cancer and liver cancer.

Item 19. The method of treating cancer of any of items 10-18, wherein the cancer is melanoma.

Item 20. The method of treating cancer of any of items 10-19, further comprising: obtaining a first sample containing or suspected of containing cancer cells from the subject prior to administering the combination of the AKT inhibitor and the WEE1 inhibitor; obtaining a second sample containing or suspected of containing cancer cells from the subject after administering the combination of the AKT inhibitor and the WEE1 inhibitor; and assaying the first and second samples for one or more markers of apoptosis, thereby monitoring effectiveness of administering the combination of the AKT inhibitor and the WEE1 inhibitor.

Item 21. The method of treating cancer of any of items 10-20, further comprising: obtaining a first sample containing or suspected of containing cancer cells from the subject prior to administering the combination of the AKT inhibitor and the WEE1 inhibitor; obtaining a second sample containing or suspected of containing cancer cells from the subject after administering the combination of the AKT inhibitor and the WEE1 inhibitor; and assaying the first and second samples for activity of a mitogen-activated protein kinase-signaling pathway, thereby monitoring effectiveness of administering the combination of the AKT inhibitor and the WEE1 inhibitor.

Item 22. The method of treating cancer of any of items 10-21, further comprising: obtaining a first sample containing or suspected of containing cancer cells from the subject prior to administering the combination of the AKT inhibitor and the WEE1 inhibitor; obtaining a second sample containing or suspected of containing cancer cells from the subject after administering the combination of the AKT inhibitor and the WEE1 inhibitor; and assaying the first and second samples for AKT dysregulation, thereby monitoring effectiveness of administering the combination of the AKT inhibitor and the WEE1 inhibitor.

Item 23. The method of treating cancer of any of items 10-22, further comprising: obtaining a first sample containing or suspected of containing cancer cells from the subject prior to administering the combination of the AKT inhibitor and the WEE1 inhibitor; obtaining a second sample containing or suspected of containing cancer cells from the subject after administering the combination of the AKT inhibitor and the WEE1 inhibitor; and assaying the first and second samples for p53 expression and/or an associated gene selected from the group consisting of: CLCA2, PVRL4, SULF2, CDKN1a, BTG2, ACTA2, TP53, FDXR, GDF15, IGFBP5 and ADAM19, wherein an increase in p53 expression and/or expression of an associated gene selected from the group consisting of: CLCA2, PVRL4, SULF2, CDKN1a, BTG2, ACTA2, TP53, FDXR, GDF15, IGFBP5 and ADAM19, is an indicator of an anti-cancer cell effect of treatment with the combination of the AKT inhibitor and the WEE1 inhibitor, thereby monitoring effectiveness of administering the combination of the AKT inhibitor and the WEE1 inhibitor.

Item 24. The method of treating cancer of any of items 10-22, further comprising: obtaining a first sample containing or suspected of containing cancer cells from the subject prior to administering the combination of the AKT inhibitor and the WEE1 inhibitor; obtaining a second sample containing or suspected of containing cancer cells from the subject after administering the combination of the AKT inhibitor and the WEE1 inhibitor, and assaying the first and second samples for FOXM1 expression and/or a expression of an associated gene selected from the group consisting of: TMPO, ANP32E, SMC4, KIF20B, ASPM, DEPDC1, NCAPG, CENPE, wherein a decrease in expression of FOXM1 and/or an associated gene selected from the group consisting of: TMPO, ANP32E, SMC4. KIF20B, ASPM, DEPDC1, NCAPG and CENPE, is an indicator of an anti-cancer cell effect of treatment with the combination of the AKT inhibitor and the WEE1 inhibitor, thereby monitoring effectiveness of administering the combination of the AKT inhibitor and the WEE1 inhibitor.

Item 25. The method of treating cancer of any of items 10-24, wherein the AKT inhibitor and the WEE1 inhibitor are administered simultaneously.

Item 26. The method of treating cancer of any of items 10-25, wherein the AKT inhibitor and the WEE1 inhibitor are administered sequentially.

Item 27. The method of treating cancer of any of items 10-24 and 26, wherein the AKT inhibitor and the WEE1 inhibitor are administered sequentially within a period of time selected from: one hour, two hours, four hours, eight hours, twelve 10 hours, twenty-four hours, 2 days. 3 days, 4 days, 5 days, 6 days and 7 days.

Item 28. A combination of an AKT inhibitor and a WEE1 inhibitor for use in the treatment of cancer.

Item 29. A combination of an AKT inhibitor and a WEE1 inhibitor for use as a medicament.

Item 30. The composition, commercial package, method or combination of any of items 1-29 wherein the AKT inhibitor is an AKT3 inhibitor.

Item 31. The composition, commercial package, method or combination of any of items 1-30 wherein the ratio of the AKT inhibitor: the WEE1 inhibitor (mole:mole) is in the range of 0.1:100 to 100:0.1.

Item 32. The composition, commercial package, method or combination of any of items 1-31 wherein the ratio of the AKT inhibitor: the WEE1 inhibitor (mole:mole) is in the range of 1:1.25, 0.15:1, 0.31:1, 0.63:1, 1.25:1, 12:1, 128:1, 16:1, 2.5:1, 32:1, 64:1 or 1:2.5.

Item 33. The composition, commercial package, method or combination of any of items 1-32 wherein the composition, commercial package, method or combination excludes a CHK1 inhibitor and/or an mTOR inhibitor.

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A composition comprising: an AKT inhibitor and a WEE1 inhibitor.

2. The composition of claim 1, wherein the AKT inhibitor is selected from the group consisting of: AZD5363, GDC0068, a combination of AZD5363 and GDC0068, a pharmaceutically acceptable salt, hydrate, amide or ester of any thereof, and an siRNA directed to AKT.

3. The composition of claim 1, wherein the WEE1 inhibitor is selected from the group consisting of: MK1775, a pharmaceutically acceptable salt, hydrate, amide or ester thereof, and an siRNA directed to WEE1.

4. The composition of claim 1, further comprising a pharmaceutically acceptable carrier.

5. A commercial package comprising an AKT inhibitor and a WEE1 inhibitor.

6. The commercial package of claim 5, wherein the AKT inhibitor is selected from the group consisting of: AZD5363, GDC0068, a combination of AZD5363 and GDC0068, a pharmaceutically acceptable salt, hydrate, amide or ester of any thereof, and an siRNA directed to AKT.

7. The commercial package of claim 5, wherein the WEE1 inhibitor is selected from the group consisting of: MK1775, a pharmaceutically acceptable salt, hydrate, amide or ester thereof, and an siRNA directed to WEE1.

8. The commercial package of claim 5, wherein the AKT inhibitor and the WEE1 inhibitor are provided as a single pharmaceutical formulation.

9. (canceled)

10. A method of treating cancer in a subject in need thereof, comprising:
administering a combination of an AKT inhibitor and a WEE1 inhibitor as a combination formulation or separately.

11. The method of claim 10, wherein administration of the combination provides a synergistic effect.

12. The method of treating cancer of claim 10, wherein the AKT inhibitor is selected from the group consisting of: AZD5363, GDC0068, a combination of AZD5363 and GDC0068, a pharmaceutically acceptable salt, hydrate, amide or ester of any thereof and an siRNA directed to AKT.

13. The method of treating cancer of claim 10, wherein the WEE1 inhibitor is selected from the group consisting of: MK1775, a pharmaceutically acceptable salt, hydrate, amide or ester thereof and an siRNA directed to WEE1.

14. The method of treating cancer of claim 10, wherein the cancer is characterized by constitutive activation of a mitogen-activated protein kinase-signaling pathway.

15. The method of treating cancer of claim 10, wherein the cancer is characterized by constitutive activation of a mitogen-activated protein kinase-signaling pathway associated with one or more mutations in BRAF, KIT and/or RAS.

16. The method of treating cancer of claim 10, wherein the cancer is characterized by constitutive activation of a mitogen-activated protein kinase-signaling pathway associated with ^{V600E} BRAF.

17. The method of treating cancer of claim 10, wherein the cancer is characterized by AKT dysregulation.

18. The method of treating cancer of claim 10, wherein the cancer is selected from the group consisting of: melanoma, colorectal cancer, thyroid cancer, breast cancer, prostate cancer, sarcoma, glioblastoma, T-cell acute lymphoblastic leukaemia, lung cancer and liver cancer.

19. The method of treating cancer of claim 10, wherein the cancer is melanoma.

20.-24. (canceled)

25. The method of treating cancer of claim 10, wherein the AKT inhibitor and the WEE1 inhibitor are administered simultaneously.

26. The method of treating cancer of claim 10, wherein the AKT inhibitor and the WEE1 inhibitor are administered sequentially.

27.-34. (canceled)
